(19) United States
(12) Patent Application Publication
(10) Pub. No.: US 2022/0184311 A1
(43) Pub. Date: Jun. 16, 2022

(54) PROSTHETIC DISORDER RESPONSE SYSTEMS

(71) Applicant: David S. Goldsmith, Atlanta, GA (US)

(72) Inventor: David S. Goldsmith, Atlanta, GA (US)

(21) Appl. No.: 17/689,880

(22) Filed: Mar. 8, 2022

Related U.S. Application Data
(63) Continuation-in-part of application No. 17/460,034, filed on Aug. 27, 2021.
Application No. 17/460,034 is a continuation-in-part of application No. 17/329,138, filed on May 24, 2021.
Application No. 17/329,138 is a continuation-in-part of application No. 14/998,495, filed on Jan. 12, 2016, now Pat. No. 11,013,858.
Application No. 14/998,495 is a continuation-in-part of application No. 17/329,138, filed on May 24, 2021.
Application No. 17/329,138 is a continuation-in-part of application No. 14/998,495, filed on Jan. 12, 2016, now Pat. No. 11,013,858.
Application No. 14/998,495 is a continuation-in-part of application No. 15/998,002, filed on Jun. 8, 2018.
(60) Provisional application No. 62/282,183, filed on Jul. 27, 2015.
(60) Provisional application No. 62/282,183, filed on Jul. 27, 2015.
(60) Provisional application No. 61/959,560, filed on Aug. 27, 2013.

(51) Int. Cl. A61M 5/172; A61M 5/14; A61M 5/142; A61M 39/02; G16H 20/17; G16H 20/30; G16H 40/63

(52) U.S. Cl. CPC A61M 5/1723; A61M 5/1408; A61M 5/14276; A61M 39/0208; A61M 2039/0211; G16H 20/30; G16H 40/63; A61M 2205/054; A61M 2210/12; G16H 20/17

(57) ABSTRACT

A fully implanted automatic disorder response system acts as a backup “immune” system, immediately detecting and dispensing an enzyme deficient or lacking due to an inborn error of metabolism, for example, in accordance with its prescription-program. In response to a disease, the remedial action is usually medicinal and/or electrostimulatory. By directly pipeline-targeting agents through pipelines from implanted reservoirs to leak-free and durable tissue connectors at the focal points of chronic disease, the system avoids the dispersion of drugs throughout the circulation and the side effects this causes, fundamentally liberalizing while optimizing the use of drugs. Electrostimulatory and other end-effectors available, each morbidity or site thereof in comorbid disease is assigned to an arm or channel of an hierarchical control system. Symptom sensors pass data up through successively higher-level microcontroller nodes to generate the cross-channel, cross-morbidity view the control microprocessor uses to command the remedial action that will optimize overall homeostasis.

### CROSS-REFERENCE TO RELATED APPLICATIONS

This application is a continuation-in-part U.S. application Ser. No. 17/460,034, filed on 27 Aug. 2021; which is a continuation-in-part of U.S. application Ser. No. 17/329,138, filed on 24 May 2021; which is a continuation in part of U.S. application Ser. No. 14/998,495, filed on 12 Jan. 2016, now U.S. Pat. No. 11,013,858, issued on 25 May 2021; which claims the benefit of U.S. Provisional Application No. 62/282,183, filed on 27 Jul. 2015. The present application is also a continuation-in-part of U.S. application Ser. No. 17/329,138, filed on 24 May 2021; which is a continuation in part of U.S. application Ser. No. 14/998,495, filed on 12 Jan. 2016, now U.S. Pat. No. 11,013,858, issued on 25 May 2021; which claims the benefit of U.S. Provisional Application No. 62/282,183, filed on 27 Jul. 2015. The present application is also a continuation-in-part of U.S. application Ser. No. 15/998,002, filed on 8 Jun. 2018; which claims the benefit of U.S. Provisional Application No. 61/959,560, filed on 27 Aug. 2013. The present application claims the benefit of these preceding applications, the entire disclosures thereof incorporated by reference in their entirety.

### BACKGROUND OF THE INVENTION

### 1. Field of the Invention

The methods and apparatus to be described are intended for prescription by internists, hepatologists, nephrologists, pulmonologists, cardiologists, urologists, gastroenterologists, gynecologists, and for use by endourologists, general, endocrine, oncological, neurological, pediatric cardiac, vascular, and cardiothoracic surgeons, interventional cardiologists, interventional radiologists, and veterinary specialists to allow 1. The automatic directly catheteric pipeline-targeted delivery of drugs and therapeutic or system maintenance substances to the sites of disease; 2. The semiautomatic control of compound bypass solid organ transplantation; 3. The semiautomatic placement of ductus segment replacement prostheses; as well as 4. The control of nondrug therapeutic devices, such as electrostimulatory, cardiac resynchronizing, thermal, and electrical assist devices in response to data transmitted to an implanted microcontroller, or in multiply comorbid disease, a hierarchical master control microprocessor executing a prescription-program responsive to data supplied by implanted sensors to continuously treat the patient while ambulatory.

### 2. Concept of the Invention

A prosthetic disorder response system is a fully implanted interconnected network of sensors, drug reservoirs, drug-releasing ductus and tissue connectors, electrostimulatory, thermal, or tool-positioning end-connectors, and catheteric drug and medicinal solution pipelines connecting the drug reservoirs to the ductus and tissue connectors, and depending upon the number of drugs and/or electrosimuation devices to be coordinated, an implanted microcontroller, master microcontroller, or in comorbid disease, a master control microprocessor to administer the prescription-program which implements the system. Sensors do not measure the concentration of drugs at the target but rather the change in symptoms attributable thereto; it is at the apical command level that drug delivery is continuously controlled, that is, where the drugs are chosen and the dose for each is set.

Unless each comorbidity in a combination of comorbities is so familiar to clinicians that the best drugs to use for each comorbidity as well as the sum thereof in most patients has already been established, the control system can be programmed to pause in order to identify the drug or durgs stored in its drug reference memory that would best respond to the immediate need, these drugs then injected into the system drug reservoirs. An automatic ambulatory prosthetic disorder response system comprises two primary components, one for control and the other consisting of end-effectors which the controller commands—by loose analogy, a brain and muscles and glands.

In monomorbid and relatively simple conditions, where the release of only a few drugs to the site of disease or its few symptoms is by direct pipeline-targeted delivery into the blood supply or the parenchymata of the affected organs, glands, or tissues, this mechanical segregation limits the interaction of the drugs with one another or with nutrients in the circulation to those passed through the same pipeline to the target. Considerably reducing the number of potential adverse reactions that might arise, where interaction is limited thus, the implanted controller is a microcontroller chip presenting an exterior surface of a tissue compatible metal such as stainless steel and free of potentially injurious projections.

More complex comorbid conditions call for a microprocessor. Broadly, isolating drugs from one another by direct pipeline-targeted delivery into the blood supplies of the diseased organs, glands, or tissues keeps these drugs out of the general circulation and eliminates them as factors in drug interactions. Moreover, withheld from nontargeted tissue, piped drugs can be delivered to the targeted tissue at concentrations higher than might be allowed to circulate and without causing adverse reactions in nontargeted tissue, especially at the higher dose used.

Directly pipe-targeting drugs into the blood supply of a certain organ, gland, or volume of tissue does not give the piped drug access to the blood elsewhere in the circulation so that the piped drug cannot affect the blood or any drugs that had been released into the general circulation except for that relatively small amount in the general circulation that enters into the blood supply of the target. If exceptionally necessary, copending application Ser. No. 15/932,172, entitled Integrated System for the Infixion and Retrieval of Implants, and Ser. No. 15/998,002, entitled Ductus Side-entry Jackets and Prosthetic Disorder Response Systems describe methods for extracting an objectionable residue in the venous drainage from gaining access to the general circulation.

And because the blood in the general circulation also flows into the blood supply of the target, piping a drug directly into the target blood supply cannot completely eliminate small-volume contact between the targeted and the circulated drugs. However, the dose level in the circulated blood entering the target blood supply tiny, and the targeted drug much more concentrated, the target should rarely become subject to more than a rare and nugatory adverse interaction. The hierarchical method used to evaluate the individual and collective efficacy of a combination of drugs to reverse the target symptom respective of each and approximate substantially normal homeostasis functions continuously in the ambulatory patient as fully implanted.

The method used to accomplish this is the same whether all of the drugs are in the circulation, or certain drugs are made to substantially bypass the circulation through isolated delivery through a pipeline directly to the target organ, gland, or volume of tissue so that the relative concentration of these drugs is much greater than that of any other drugs that enter the structure through its blood supply, or all of the drugs are passed together through a pipeline to the target structure, or the routing of drugs includes all of these methods.

That the relatively large dose of the pipe-targeted drug is denied access to the highly dilute drug or drugs in the circulation except for that passing into the blood supply of the target should be sufficient to prevent any mix therebetween from attaining the threshold volume essential for most if not all otherwise potentially problematic drug-drug interactions to arise. In point of fact, even this negligible consequence is easily avoided simply by deferring the release of other drugs into the general circulation until the time to clearance for the targeted drug or drugs has passed. This factor should considerably liberalize the simultaneous use of numerous drugs that previously had to be withheld from simultaneous administration due to concerns over adverse reactions.

In practical cases of organ failure, a single drug often will not suffice. Then the ability to prescribe a combination of drugs at higher doses than would be released into the circulation made possible by direct pipeline targeting is taken advantage of by releasing all of these drugs through the same pipeline. Except that each drug is more concentrated than were it released into the circulation, the problem of optimizing the relative doses among these to obtain the best outcome and minimize if not eliminate any adverse interactions is no different than that pertaining to drugs, albeit in lower concentrations, compresent in the circulation.

An automatic and fully implanted prosthetic disorder response system is intended to function as a backup ‘immune’ system able to detect, monitor and treat any abnormal condition known to internal medicine for which evidence-based pharmaceutical and/or electrostimulatory therapy has been established. As such, the system is preferably fully or closed-skin implanted, only an externally placed body surface port and/or an unusually large number of drug reservoirs, for example, exceptionally relegated to a paracorporeal body pack, and then only when complex comorbid disease makes an inordinate number of components necessary and/or a urine outflow opening necessitates the use of a paracorporeal urinal or collection bag.

A listing of applications from least to most advanced comprehends:

1. An isolated line without automatic control. A subcutaneously implanted body surface port with self-sealing cover membrane leading into a central or non-central organ blood supply drug delivery line connected by means of a ductus side-side entry jacket or vascular valve to remain in place indefinitely to allow immediate vascular access into the target artery in the clinic, such access allowing infusion of insulin, for example, directly into the portal vein, or an antipsychotic directly into an internal carotid, for example, manually on a discretionary basis.

Directly pipeline-targeted thus, the drug is isolated from the general circulation, so that any side effects and/or drug interactions are constrained to the tissue targeted. A central line or a line targeting the blood supply of a specific organ, gland, or volume of tissue can be implanted along with an implanted drug reservoir and a sensor or sensors to indicate the need for an automatically released dose; however, most applications for a prosthetic disorder response system are directed toward more complex conditions requiring the automated detection of the need for and the dispensing of a drug or drugs.

2. The same situation as in item 1 except that the port incorporates a protected opening to the exterior for insertion of a miniature cabled device such as a scope, laser, or intravascular ultrasound probe, for example, such access allowing the insertion of a ureteroscope, for example, directly into a ureter from a superior level along the urinary tract, thus avoiding an infected urinary bladder and allowing image recording and/or the release of one or more drugs from this position. The risk of spreading an infection when moving through an infected bladder and up into a ureter as forcing the postponement in an essential follow-up procedure is addressed with references cited below. When independent of a line to directly target a drug, automatic control is uninvolved and placement is often accomplished with the aid of a hand-held ultrasound device, chest radiographs, and fluoroscopy.

3. A connection such as that described in item 1 above wherein the subcutaneous body port is connected to the target ductus with the addition of a drug reservoir having a pump at its outlet controlled by an implanted microcontroller on the basis of data received from an implanted sensor positioned to monitor the target. Such an implanted automatically microcontroller-controlled line eliminates the need to depend upon a prescription-oblivious or adverse patient such as an infant or one senescent for adherence to their prescription.

Another application is the automatic direct release into the internal carotid or carotids of an uncooperative patient of a neuroleptic, or antipsychotic drug whether scheduled or in response to a sensor-detected psychotic episode. Directly pipeline-targeted thus, the drug is isolated from the general circulation, so that any side effects and/or drug interactions are constrained to the tissue targeted. However, because the brain is itself the seat of the adverse side effects caused by the drugs targeted to it, a sequela such as tardive dyskinesia responsive to an antipsychotic would not be avoided. To accomplish that would necessitate an ability to target and thus isolate the delivery of drugs within the brain.

For an adverse or potentially adverse condition which the patient does not sense, such as hypokalemia when this bodes susceptibility to a stroke, the deficit can be corrected by such an automatically controlled drug targeting pipeline. In the case specified, a blood potassium sensor is used to signal the implanted microcontroller to release potassium from the implanted reservoir into the general circulation (see, for example, Judge, C., O'Donnell, M. J., Hankey, G. J., Rangarajan, S., Chin, S. L., and 32 others 2021. “Urinary Sodium and Potassium, and the Risk of Ischemic and Hemorrhagic Stroke (INTERSTROKE): A Case-Control Study,” American Journal of Hypertension 34(4):414-425; Vinceti, M., Fillippini T., Crippa, A. deSesmaisons A., Wise, L. A., and Orsini, N. 2016. “Meta-analysis of Potassium Intake and the Risk of Stroke,” Journal of the American Heart Association 5(10):e004210; Seth, A., Mossavar-Rahmani, Y., Kamensky, V., Silver R., Laksminarayan, K. and 3 others 2014. “Potassium Intake and the Risk of Stroke in Hypertensive and Non-hypertensive Women in the Women's Health Initiative,” Stroke 45(10) 2874-2880). Such isolated lines, unless multiple, do not necessitate

An equally important function is the monitoring of blood sodium level in a hypertensive at high risk for cardiovascular disease (see, for example, Cogswell, M. E., Mugavero, K., Bowman B. A., and Frieden, T. R. 2016. “Dietary Sodium and Cardiovascular Disease Risk—Measurement Matters,” New England Journal of Medicine 375(6):580-586). Most patients need only be made aware of the condition to reduce their dietary intake of sodium. Otherwise, a blood sodium sensor is used to signal an implanted microcontroller to release a diuretic (see, for example, Ellison, D. H. and Felker, G. M. 2017. Diuretic Treatment in Heart Failure,” New England Journal of Medicine 377(20): 1964-1975).

A second independent drug delivery controlled line with sensor can be used to continuously indicate and adjust the blood pressure, thus controlling the dose of the diuretic, loop diuretics such as furosemide only used when unavoidable and then requiring stringent control as ototoxic as well as posing risks for many other serious adverse effects to include potentially dangerous electrolyte imbalances (see, for example, Khan, T. M., Patel, R., and Siddiqui, A. H. 2021. “Furosemide,” Treasure Island Fla.: StatPearls Publishing, online; Jackson W., Taylor, G., Selewski, D., Smith, P. B., Tolleson-Rinehart, S., and Laughon, M. M. 2018. “Association between Furosemide in Premature Infants and Sensorineural Hearing Loss and Nephrocalcinosis: A Systematic Review,” Maternal Health, Neonatology, and Perinatology 5:13; Ding, D., Liu, H. Qi, W., Jiang, H., Li, Y., and 4 others 2016. “Ototoxic Effects and Mechanisms of Loop Diuretics,” Journal of Otology 11(4):145-156; Ryback, L. P. 1985. “Furosemide Ototoxicity: Clinical and Experimental Aspects,” Laryngoscope 96 (9 Part 2 Supplement 38:1-14).

4. Evaluation of the coordinated release of drugs whether pipeline-targeted. Directly pipeline-targeted drugs delivered without other drugs having been released into the circulation are monitored for the need to adjust the dose of any one or more drugs in terms of their individual and combined effect where brief stoppages in the delivery of each makes it possible to gauge their interactions. The omission of nonessential drugs is always beneficial in reducing control complexity, the number of microcontroller nodes needed, and system expense. While conditions of multiple organ failure and complex comorbidity will force system complexity, the ultimate goal of system design is use of the fewest drugs in the smallest doses with optimal system simplicity for the problems to be treated.

With all drugs pipeline-targeted, exposure to nontargeted tissue of the drugs isolated thus and their side effects are eliminated. If drugs are also released into the circulation, then the relative proportion of these compared to the drug targeted in the blood reaching the target will be considerably less, perhaps enough so that the side effects any of the other drugs cause will be rendered inconsequential. Any mixture of drugs, however, must be monitored for drug interactions and side effects. Accordingly, because it removes the complications of drug interactions and direct side effects to tissue other than that targeted, pipeline targeting not only liberalizes the use of drugs but imparts simplicity and reduced cost.

Moreover, simplification thus facilitates the development of standardized control programs requiring little if any additional revision or development work to deal with commonly encountered disorders with relatively little addition of further expense. Standardized and fully tested systems to deal with common cases of comorbidity to include both software and hardware can be packaged with programmed microprocessor with all of the parts needed without additional design work.

The development of such prepackaged hardware and software prosthetic disorder response systems moves from the simple to the more and more complex. In the simplest case, a microcontroller controls the release of a single drug through a central line connected to the substrate ductus by means of ductus side-entry jacket or vascular servovalve into the circulation into which no other drugs have been released. The control system modulates the release of the drug in response to the continuous feedback to the microcontroller of the symptomatic indicia indicative of the extent of deviation reflexted by the symptoms from the desired end point.

The next step in development is then to use the same hardware to apportion dosing among two or more drugs released through the central line and/or having been released into the general circulation. Rather than apply such single line feedback to situations where plural drugs can interact and produce side effects, a control system is needed that can process the sum of sensor inputs in comorbid disease to achieve the best cross-drug cross morbidity condition for the entire set thereof. For this reason, comorbid, and especially complex comorbid disease is best approached with the aid of a hierarchical control system addressed farther below.

With a prepackaged system that includes hardware, software, and instructions for implanting, the clinician then need only determine that the disease of the individual patient is sufficiently consistent with commonly encountered cases of comorbidity of the kind for which the packaged system was designed so that the system will be able to detect, execute, and spontaneously ‘learn,’ just what adjustments in the drug regimen and/or electrostimulation best reduce the symptoms.

While developed in support of areas other than pharmacological practice, the design of the circuitry to implement such self-functioning, as well as the hierarchical control essential to support more complex systems as addressed farther along, is by now well established. A higher step in development would not be limited to sensors deployed to monitor known indicia of known disease but employ numerous tiny sensors to identify and delivery means able to respond to a range of symptoms (see, for example, Sutskever, I. 2013. “Training Recurrent Neural Networks,” Doctoral Dissertation, University of Toronto, Toronto, Canada; Hirashima, M. and Nozaki, D. 2012. “Learning with Slight Forgetting Optimizes Sensorimotor Transformation in Redundant Motor Systems,” Public Library of Science Computational Biology 8(6):e1002590; Franklin, D. W., and Wolpert, D. M. 2011. “Computational Mechanisms of Sensorimotor Control,” Neuron 72(3):425-443; Huh, D. and Todorov, E. 2009. “Real-time Motor Control Using Recurrent Neural Networks,” Institute of Electrical and Electronics Engineers Symposium on Adaptive Dynamic Programming and Reinforcement Learning, online at ieeexplore.ieee.org; Liu, D. and Todorov, E. 2009. “Hierarchical Optimal Control of a 7 DOF Arm Model,” Institute of Electrical and Electronic Engineers Symposium on Adaptive Dynamic Programming and Reinforcement Learning, pages 50-57; Barto, A. G. and Mandevan S. 2003. “Recent Advances in Hierarchical Reinforcement Learning,” Discrete Event Dynamic Systems 13(4):341-379; Loeb, G. E., Brown, I. E., and Cheng, E. J. 1999. “A Hierarchical Foundation for Models of Sensorimotor Control,” Experimental Brain Research 126(1):1-18; Dietterich, T. G. 1998. “The MAXQ Method for Hierarchical Reinforcement Learning,” ICML [International Conference on Machine Learning] 1998:118-126; Nguyen, D. and Widrow, B. 1990. “The Truck Backer-Upper: An Example of Self-learning in Neural Networks,” Proceedings of the International Joint Conference on Neural Networks, in Eckmiller, R. (ed.), Advanced Neural Computers, Amsterdam, Holland: Elsevier/North-Holland, pages 11-19).

However, the addition or omission of any drug and the advent of one or more pertinent new drugs can invalidate such a standardized system. For this reason, changes in the prescription regimen of nugatory potential to upset the basic system in any significant way can be made available as extensions to the basic program. When one target is primary and another secondary, such as the heart and kidneys in cardiorenal syndrome, where the correction of kidney function follows from that of the heart without the need to separately treat the kidneys, a second pipeline to the kidneys is omitted. If the secondary condition warrants separate medication, the targeted drugs are first evaluated separately then as combined.

One or more sensors continuously indicating the dose-to-effect the dose is adjusted for optimal efficacy. Delivered in isolation thus, drugs are prevented from significant interaction with each other but not with drugs in the general circulation. When one or more other drugs had been released into the circulation, both pipeline-targeted and nontargeted drugs should undergo a running tally to optimize the efficacy of each in terms of their combination. Where no drugs had been released into the circulation, two pipeline-targeted drugs can be monitored to set the optimal dose of each within the context of the two together.

Where more concentrated doses of drugs are pipeline-targeted to different targets, to include a chemotherapeutic to a primary malignant tumor and a metastasis thereof, and a background dose is released into the circulation to destroy any cells that may have been shed, the combination of drugs to include both those targeted and those nonchemotherapeutic circulated is evaluated to determine the optimal dose for each drug within the combination by a hierarchical control system. The same applies whether the drugs are delivered by pipeline targeting or release into the circulation. Such a continuous evaluation of individual drug efficacy and the need to adjust it dose or replace it with another drug can be accomplished with the aid of a hierarchical control system.

The system evaluates the efficacy of each drug, then this data passed up to a higher level where the drugs are evaluated in combined use, and this pattern continues to pass up the hierarchy until the optimal instantaneous proportional doses among the drugs is clarified. In this way, an adverse reaction and the drug responsible for it is immediately identified. If pipeline-targeted and under the immediate control of the system, the responsible drug can be reduced in the rate of dose delivery or stopped. If the system had not dispensed that drug, a signal is generated indicating the need to reduce or replace it.

In this way, the system can continuously adjust the relative proportion in the mix of each targeted drug and call for adjustment in drugs released into the circulation so that the combination of drugs will remain optimally efficacious. Control system design becomes more complex and expensive in proportion to the number of drugs released into the circulatory system at the same time where no segregation of any drug or drugs from the others has been provided to preclude adverse interactions. Each symptom, or pathophysiologically relevant indicator, due to a comorbidity is assigned a sensor which feeds its data to a microcontroller at the lowest node for coordination with those assigned to other symptoms.

These microcontrollers then feed their data up to the next higher level where the data for the sum of symptoms appurtenant of one in a number of comorbidities is determined. At the overall control level, the master control microprocessor coordinates the data for the set of comorbidities. The number of symptoms appurtenant of each comorbidity determines the number of sensors, nodes, and programming essential to ascertain the best combination of drugs and doses from among those provided or which should be provided.

Because the control system monitors the consequence for each symptom of each comorbidity due to each drug from the sensor node up through the next higher level of symptom inclusivity and so on, the origin of any adverse interaction is immediately identifiable to its source in the addition of another drug. The number of levels in the hierarchy determined by the number of symptoms and the need to determine the optimal medicinal and/or electrostimulatory response most effective to treat the combination of these, employment of the system is made simpler and more affordable when the prescribing clinician has the knowledge and experience to omit symptoms which can be regarded as incidental.

The functioning and presence of the system is intended to remain oblivious to the patient whose mobility is unaffected by it. The main object in such a system is to counteract one or more out-of-range metabolic states upon inception through immediate detection by continuously monitoring implant sensors before the patient even senses a problem much less experiences ill health due to the defect. The identity of the defect is not lost, however, as it is documented by a data recorder. The information reported by the sensors is passed to the implant controller—in comorbid disease, a microprocessor—which commands the delivery of remedial medication and/or electrical discharges to, and/or changes in temperature at the site.

This process is intended to proceed with the patient oblivious to it, that is, before any symptoms rise to the threshold of sensation. For diagnostic purposes, the character of the occurrence, the response made to it, and the result are continuously documented by an event recorder. Such a system can be programmed to fully administer surgical procedures such as a solid organ transplant or the replacement of a segment of a larger vessel, and support an extracardiac transposition of the great vessels, in no case necessitating a stoppage of blood flow.

Since a piped drug in the blood reaching a target organ or gland is only increased as a relative proportion of the blood sum drug content, piping as a factor is disregarded in gauging deviations from normal at the target as affect homeostasis of the body as a whole; all targets, piped or not, are monitored and evaluated alike. Sensors are assigned to monitor the symptoms associated with each morbidity. The instant deviation from normal of pertinent physiological indicia are detected and a hierarchical, pyramidally, or tree-organized, control system used to detect and respond to such deviations. The process of evaluating and responding to deviations within the context of homeostasis overall commences locally at the target.

At the bottom of the tree, sensors are directed toward one or more symptoms of one in a number of comorbidities, and the ground level microcontroller nodes to which the sensors provide input coordinate this data to determine the best drug to respond to that morbidity as an arm, branch, or axis of the system.

Moving up the tree, the microcontrollers integrate data representative of the other comorbidities to determine which drugs will best serve to treat the sum of comorbidities. Accordingly, the number of microcontroller node levels corresponds to the number of comorbidities or the symptoms thereof. This process of rising expanded inclusion progresses up through the tree to the master control microprocessor which issues therapeutic commands based upon the sum of morbities.

The distinction between axes which is exclusively the case at the ground level thus becomes more and more obscured moving up through the levels of nodes as more and more other comorbidities are taken into account by integration into the determinative process. That is, sensor data initially reports out of normal range data and thereafter continuously indicates the effect of the corrective or remedial measures taken by the master controller. The progressively more inclusive data moves up through the tree of microcontroller nodes each consecutive level of which integrates the less target-inclusive data sent to it by the nodes at the next lower level in the pyramid, or tree.

That is, the data reported by microcontroller nodes at subordinate levels is continuously passed up through the more inclusive microcontroller nodes to the master control microprocessor to determine the remedial action at each target essential to return the sum thereof to the values most closely indicative of normal homeostasis, and the remedial commands of the master control microprocessor continuously pass back down through the tree to the end-effector or effectors assigned to the same targets as the sensors which sent the data, control thus by negative feedback throughout the array, at every microcontroller node, the master control microprocessor, and thus, the tree as a whole.

When drug or electrostimulatory therapy is targeted, the local sensor or sensors and controller continuously gauge the proximity to the desired end point. In comorbid disease, with drugs released into the general circulation, this continuous cyclical feedback moves up through the pyramid to successively higher microcontroller nodes at each successively higher level cross-evaluate the data received from subordinate nodes closer to the point of treatment, thence to the master control microprocessor, which returns the continuous stream of commands for adjusting the dosing back down through the pyramid to the end effectors respective of the sensors.

The master control microprocessor commands the release of drugs to each of the various targets so that the sum thereof most closely approximates if not produces normal homeostasis for the combination of drugs and electrical discharge stimulators made available to it. An even closer approximation would require replacing one or more of the drugs with others. Which to replace will be apparent from the morbidity arm (axis, channel) that falls short of its desired endpoint. That diagnostic accuracy of the system would be thrown off due to the omission of an essential arm or axis of one comorbidity is implausible.

As indicated, because it removes the targeted drug from drug-drug interactions and exposure to unintended tissue, the isolation of drugs by direct pipeline delivery into the blood supply or parenchyma of organs, glands, and volumes of tissue targeted enables the simultaneous use of drugs long known and those yet to be discovered that previously had been disallowed. Furthermore, by avoiding dilution in the circulatory system, direct pipeline targeting makes possible the considerable reduction in the volume of doses; not only can the concentration of familiar drugs previously disallowed in the required concentration for optimal efficacy be increased when simultaneously released into the general circulation, but the use of costly new drugs may be made practicable, and drugs disallowed due to cost in less developed parts of the world may be allowed.

If disease is multiply comorbid and symptomatic so that the number and/or size of the system components is exceptionally large, the larger and/or additional number of components is relegated to a belt-worn body pack. Instantly responsive, the system detects the analytes associated with and strikes down a genetically transmitted or predisposed disorder before the patient experiences symptoms. That such a system is easily enabled to transmit data to a clinic by medical telemetry and that implanted power sources can be replenished by transcutaneous energy transfer is addressed in copending applications specified in the section above entitled Cross Reference to Related Applications.

FIG. 1 provides a schematized, or nonanatomical, depiction of such a fully implanted system, the only paraorporeal rather than implanted component being a urine drainage collection bag, or urinal, cinched about the ipsilateral thigh as the urine outlet pipe of a prosthesis such as those shown in FIGS. 28 and 30 of copending application Ser. No. 16/873,914, entitled Vascular Valves and Servovalves—and Prosthetic Disorder Response Systems. The end-effectors are integral with or irreversibly connected to end-connectors that must never leak, migrate, or otherwise lose the integrity of the junctions with the tissue to which these are connected in order to deliver medicinals, blood, or to pass urine.

Most end-effectors are ductus side-entry jackets, described and illustrated in copending application Ser. No. 15/998,002 entitled Ductus Side-entry Jackets and Prosthetic Disorder Response Systems which connect catheteric drug, blood, or urine moving lines to other ductus or tissue. When flow is to be switched from one outlet passageway to another, the end-effector is a highly dampled nonsparking solenoid driven vascular valve. When flow must be continuously and accurately proportioned between two outlets, the end-effector is a vascular servovalve. Vascular valves and servovalves are described and illustrated in detail in copending application Ser. No. 16/873,914, entitled Vascular Valves and Servovalves—and Prosthetic Disorder Response Systems.

Thereafter, the system will monitor and automatically support and treat the procedure through the programmed release of medication in accordance with the control microcontroller or microprocessor prescription-program, as well as detect and immediately respond to any sequelae according to the program. Moreover, except with a pediatric, disturbed, or senile patient who will not remain motionless during the procedure, the need for general, rather than local or regional anesthesia with its potential complications can be avoided.

The avoidance of a need for repeated revisions in a child already frail a signal objective, for pediatric patients born with a malformed heart or other organ which using conventional methods can be no more than inadequately repaired, or with any of the numerous disorders of amino acid or organic acid metabolism, for example, the drug and bloodlines used must be capable of accommodating growth. Fabric to provide a high degree of expansion consistent with maintaining integrity is described in copending application Ser. No. 16/873,914, entitled Vascular Valves and Servovalves—and Prosthetic Disorder Response Systems.

To be capable of responding to any disorder for which remedical measures are available, the system must be capable of access to any point and depth in the body exclusive of neighboring tissue using end-connector/effectors which are stable, migration and leak-free, durable, devised for minimal foreign body irritation, and provided with one or more service or accessory channels to deliver anti-inflammatories to counteract inflammation and antimicrobials to counteract infection. End-connectors must be capable of permanent fixation about a ductus, tissue surface, as well as to a tissue surface in order to hold the tip of a styliform device in a fixed position both in depth and angle to maintain aim at a target in the parenchyma of an organ, within tissue, or a gland. In FIGS. 13A thru 16, U.S. Pat. No. 11,013,858 describes and illustrates means that allow a styliform device to advance as its target, such as a tumor, recedes.

Vascular valves and servovalves, described and illustrated in copending application Ser. No. 16/873,914, entitled Vascular Valves and Servovalves—and Prosthetic Disorder Response Systems, do not represent simply passive combined flow through connector junctions, but can apportion outflow between either of two outlet passageways, making these indispensable for compound/bypass solid organ transplantation, as described and illustrated in copending application Ser. No. 16/873,914.

Solenoid driven vascular valves transfer all flow from one outlet passageway to the other suddenly, whereas servomotor driven vascular servovalves transfer flow from one outlet to the other in a gradual manner under continuous control which can vary between very slow or very fast. When the viscosity of the drug to be dispensed is higher, drug reservoirs, usually implanted subcutaneously in the pectoral region, have a small peristaltic pump at the outlet, and the contents may be propelled ahead of a column of water. Low viscosity drugs are gravity fed, the controller working a tiny solenoid-driven stopcock at the outlet.

Electrostimulatory end-effectors include nonjacketing side-entry connectors with electrified anchoring needles as described and illustrated in U.S. Pat. No. 11,013,858, which can be microprocessor-programmed to discharge in any pattern and not only fix the terminous in place, but these needles hollow as well, can be used to inject medication into the substrate tissue apart from or in combination with electrostimulation. Nonjacketing side-entry connectors can also mount and fix in position miniature styliform tools such as commercial microelectrodes used for deep brain stimulation or excimer lasers, where a shift in position of the working tip would misdirect the treatment. All connector types can be radiation shielded to allow the use of low to medium dose rate radioisotopes.

To coordinate the action of commercial electrostimulatory neuromodulators, pacemakers, cardioverter defibrillators, combination pacemaker/cardioverter defibrillators, and other electrical therapeutic implants with the release of drugs, the pertinent instructions are incorporated into the prescription-program. Unlike such exceptional extensions of control to include commercial products, the coordination of drug release through the hollow injection needles in nonjacketing side-entry connectors in coordination with the patterned electrical discharges delivered through the same needles is unexceptional.

Also unexceptional is the application to the drug or drugs being released through the hollow and electrified anchoring needles of a nonjacketing side-entry connector of heat with a built in insulated thermal wire or of chilling with a built in cooling Peltier thermoelectric heatsink (Peltier device, solid state refrigerator, thermoelectric cooler—see, for example, Zhao, D. and T. Gang, T. 2014. “A Review of Thermoelectric Cooling: Materials, Modeling, and Applications,” Applied Thermal Engineering 66(1-2):15-24; Taylor, R. A. and Solbrekken, G. L. 2008. “Comprehensive System-level Optimization of Thermoelectric Devices for Electronic Cooling Applications,” Institute of Electrical and Electronics Engineers Transactions on Components and Packaging Technologies 31(1):23-31; Venkatsubramanian, E., Siivola, E., Colpitts, T., and O'Quinn, B. 2001. “Thin-film Thermoelectric Devices with High Room-temperature Figures of Merit,” Nature 413(11): 597-602).

The coordination of electrostimulatory deep brain microelectrode discharge with the release of psychotropic medication such as anxiolytics, or tranquilizers, antipsychotics, or neuroleptics, antidepressants, dissociatives, nootropics, or somnolents, stimulants, and opioids, into either or both internal carotids through ductus side-entry jackets falls well within the capability of the control system. The stereotactically positioned microelectrode does not require fixation in place with the aid of a nonjacketing side-entry connector. A third category of components—controller support devices—comprise a rechargeable lithium ion or newer technology battery as power source, transdermal, or transcutaneous, battery charging secondary coil or antenna for transcutaneous energy transfer, and the hardware and software essential for secure wireless data transmission.

New capabilities fostering the development of new technology, a fully implanted disorder response system will often incorporate features that remain unused until the newer techniques these make apparent have been realized and developed. The concurrent targeting of a lesion electrically and with a drug or drugs delivered either or both through the circulatory system whether using docking, or homing, or susceptible carrier-bound superparamagnetic nanoparticulate technology as opposed to unconstrained dispersal throughout the circulation, or through the proximate blood supply, or using these methods in various combinations is but one example.

Docking is addressed below, while magnetically based targeting is addressed in copending application Ser. No. 15/932,172, entitled Integrated System for the Infixion and Retrieval of Implants. Any similarity of conventional technology to the kind of implanted prosthetic disorder response system addressed here is misconceived on the basis of partial and superficial resemblance, and to equate a system of the kind intended to any system or system components in use reflects a lack of awareness as to the critically superior diagnostic and therapeutic capabilities a system of the kind intended can provide.

For example, existing means for communicating with the body interior such as central and cannulated intravenous lines cannot be permanently connected to the blood supply of a specific organ, gland, or volume of tissue in the body, and cannot be fully, or closed-skin implanted, much less remain functional without injury to the patient. In contrast, a permanent line connected by a ductus side-entry jacket or vascular valve can remain in place indefinitely and deliver medication into the general circulation or—and this is a significant distinction—directly into the blood supply of any specific organ, gland, or volume of tissue.

Equally important, whereas a central line with connections made according to the guidelines set forth in the applications specified above are designed to allow growth from infancy, provide at least one service or accessory channel to release an anticoagulant or antimicrobial into the line and its connection, and therewith to remain in place until the end of life, the maximum time a conventional connected central line can remain in place before it causes injury, and/or becomes infected or otherwise disabled is about a half year (see, for example, Gonzalez, R. and Cassaro, S. 2021. “Percutaneous Central Catheter,” Treasure Island, Fla.: StatPearls Publishing, online; Duwadi, S. Zhao, Q., and Budal, B. S. 2019. “Peripherally Inserted Central Catheters in Critically Ill Patients—Complications and Its Prevention: A Review,” International Journal of Nursing Sciences 6(2:99-105/2021 8(1):IV; Cotogni, P., Barbero, C, Garrino, C., Degiorgis, C., Mussa, B., De Francesco, A., and Pittiruti, M. 2015. “Peripherally Inserted Central Catheters in Non0hospitalized Cancer Patients: 5 year Results of a Prospective Study,” Supportive Care in Cancer 23(2):403-409). That the materials of which the lines and connectors are made include anti-thrombogenic and antimicrobial surface treatments is considered superfluous.

The control and diagnostic instruments that are connected to in order to use such lines are not implanted. The ability to isolate any given blood supply for connection using any of the specially devised ductus and tissue connectors described in the foregoing applications to deliver a drug, for example, into that lumen while excluding the rest of the circulatory system, and to do so with a jacket contact interface which unlike any other, not only allows the connection to remain in place indefinitely, while providing support medication through an service or accessory channel in the connector to dispel irritation, but allows the direct pipeline targeting of drugs to a specific organ or volume of tissue on a discretionary selective basis. From a medical standpoint, segregated delivery of drugs in this directly pipelined manner provides numerous distinct and consequential advantages.

This selectability liberalizes dosing in that drugs directly targeted thus can be more highly concentrated than would be allowed to circulate, and reciprocally, targeted doses need not be large enough to withstand dilution throughout the circulatory system. This can make possible the extended use of critically more effective but otherwise unaffordable newer drugs. In comparison, to place a subcutaneous portacath, subdermally tunnel the drug delivery line to the ductus side-entry jacket or nonjacketing side-entry connector, and fix the this connection in place is a one-time endoscopic procedure which can be accomplished at less expense.

Except that it is smaller, a subcutaneously placed body surface port with multiple needle insertion-openings each leading into a different delivery catheter (drugline, drug feedline) as described and illustrated in FIGS. 26A and 26B in copending application Ser. No. 16/873,914, entitled Vascular Valves and Servovalves—and Prosthetic Disorder Response Systems, is much the same as a commercial portacath (mediport, injection port) in that it is usually placed in the pectoral region, is entered through self-sealing silicone membrane, and during periods of disuse, is filled with heparinized saline to prevent the buildup of clot. With respect to FIG. 26B where relatively few drugs are needed, separate openings identified epicutaneously, or on the overlying skin, by tiny tattoos allows a high degree of clarity to avoid human errors and allows the port to less cause a protrusion to view.

An alternative embodiment to that shown in FIG. 26B emulates a turret such as shown here in FIG. 4. It allows a large number of low volume drugs to be stored in ampules inserted into a turret much as are rounds in the cylinder of a revolver for rotation into the angle for replenishment. By rotating the turret, this arrangement allows the sequential replenishment of several drugs through the same opening. Involving complexity and expense to assure the precision required to control a turret that is unseen, not allowing quick drug replenishment with a multiple syringe or jet injector such as those shown in FIGS. 27A and 27B, and tending to cause the skin overlying the port to protrude more as a cosmetic deficit, such an embodiment is dispensed with in favor or using a multi-opening miniature port with the number of openings needed.

The smallest caliber, or gauge, of vessel to which a ductus side-entry Jacket can connect a line is limited only by the precision micromachining capability of the manufacturer. However, blood supply and drainage vessels of pathophysiological and therapeutic importance tend to be relatively large in caliber, and even in an infant, or in veterinary practice, a small mammal, whose vessels are tiny, unless its other branches supply structures that must be avoided, the target can be accessed upstream through the larger vessel of which the vessel intended is a branch.

Accordingly, a specific organ, gland, or volume of tissue affected by a chronic disease can be targeted to receive medication on a scheduled basis without having to disperse the medication throughout the body to arouse adverse drug interactions and/or side effects at the cost of wasting most of each dose through dilution throughout the circulatory system. This selective capability represents a substantial advancement in eliminating exclusively interfering contextual complications having nothing to do with either the target or the drug. At the same time, nonjacketing side-entry connectors make possible extravascular connection to any depth into a volume of tissue which can remain in place indefinitely.

Detailed information concerning vascular jackets and tissue connectors is provided in the applications specified above in the section entitled Cross Reference to Related Applications. A major function of a prosthetic disorder response system is the scheduled release of a drug or drugs on the basis of sensor feedback as prescribed by the prescription-program. Electrical and fluid lines connecting the implanted system to apparatus outside the body severely hinder if not obviate unimpeded ambulatory movement and have limited if any usefulness outside the clinic. Implanted sensors signal the need for and continuously report the result of providing an essential drug or drugs.

Copending application Ser. No. 16/873,914, entitled Vascular Valves and Servovalves—and Prosthetic Disorder Response Systems, addresses sensors to detect bacteria, viruses, tumor cells, sundry infectious pathogens, diseases and symptoms thereof, analytes indicative of graft organ rejection, low or high density lipoprotein, inflammation and the mediators thereof, analytes signaling an incipient or emergent crisis such as a myocardial or cerebral infarction, to include prodromal hypoxia and nutrient deprivation. Also detectable are a pathogen-induced integrated stress response and cytokine storm. Virtually any metabolite or bodily substance to include many not mentioned here can now be detected with the aid of a sensor.

Microcontroller and microprocessor components and chips are selected for small size, least weight, and much as conventional heart synchronizers and electrostimulation devices, are suitably encapsulated to prevent enzymatic or hydrolytic breakdown. Sensors are encapsulated to discourage the formation of an obstructive fibrous coating, and side-entry jackets and vascular valves are lined with a relatively thick layer of parylene coated viscoelastic polyurethane foam to invest rather than compress the tiny vasa nervora and vasa vasora along the adventitia of a larger substrate vessel that if constricted would immediately initiate atherosclerotic degradation in the substrate vessel, which also to prevent, jackets and vascular valves are perforated through and through to allow exposure to the surrounding milieu.

The relatively thick lining allows applying the jacket or valve to vessels over a range of diameters and allows for growth in a pediatric patient, making certain critical applications to be accomplished in a child already impaired without the need for multiple revisions. For any but the simplest applications, one might argue for a large number of sensors to monitor exceptional symptoms. However, limiting the number of sensors to those directed to characteristic rather than rare symptoms of the condition or conditions treated will considerably reduce the cost and programming complexity of the system.

The single most important criterion governing the design of such a system to make it optimal as well as practicable and affordable is simplicity. Sensors are directed toward key symptoms—those most characteristic and pronounced and those associated with frequent side effects. Except that a disorder reliably predictable on the basis of known medical history and/or genetic information may recommend the preplacement of responsive components, sensors, drug reservoirs, druglines, and the prescription-program, system design is best dealt with as are drugs—the fewest in the smallest dose that work is best.

Where no drugs are to be directly pipelined into the general circulation, but rather isolated from one another by direct pipeline-targeted delivery into the blood supply of different organs or glands, programming the master controller of a hierarchically controlled prosthetic disorder response system is simplified to the extent that adverse drug-drug interactions and rare side effects can be omitted from the program. However, common sequelae which could prove severe may justify the added complexity and expense of the system.

Sensors are selected for those key indices of a present or predictable chronic condition subject to progressive or episodic change, with others generally discounted. Due to differences in practice style based upon the experience of individual diagnosticians, it is best that more than one be involved in this process. The diagnostician should be familiar with the application of cognitive shortcuts (see, for example, Mark, D. B. 2005. “Decision-making in Clinical Medicine,” in Harrison's Principles of Internal Medicine, New York, N.Y.: McGraw-Hill, 16th Edition, pages 6-13). Decision trees in one or another form have been used in medical diagnosis forever, the distinction here being that such logic is incorporated into software and hardware which can be fully implanted in the patient.

Any organ can be the primary or a sequelary site of disease, the difference between kidney disease and kidney involvement as secondary due to dependency upon the normal functioning of the heart a common example. Patients who can be depended upon to adhere to a prescription can make it possible to limit the number of lines for automated treatment to insidious or ‘silent’ secondary symptoms. Given this consideration, whether organs that would quickly respond with treatment of the primary disease should be directly treated by the implanted automatic prosthetic disorder response system must be a clinical judgment.

Generally, if and only if remedial treatment of the primary cause of disease will dependably dispel that secondary or subsidiary, that secondary is not provided with sensors and means for the direct pipeline delivery of drugs. Unless strategically limited to the detection of and response to the pathophysiological indicia underlying and associated with the disorder or combination of disorders known or most likely to affect the specific patient, the potential number of metabolic and immunological processes requiring to be monitored and the number of drugs to be prepositioned in order to respond to these could immediately render the system—at least in an unnecessarily elaborate form—impracticable and unaffordable.

Reduction to practice thus commences with an analysis of updated medical records, medical history, genetic testing, and a thorough physical examination. Symptoms associated with a disorder likely to be sensed and reported by the patient should defer to the processes underlying the sensible consequences, which will usually precede the generation of an experiential correlate, that is, appear to consciousness. Thus, the implanted system equipped to detect such underlying changes creates the capability of commencing treatment immediately, perhaps preventively, before the patient even becomes aware of the condition. In some cases, such as incipient pancreatic and lung cancer, this silent premonitory prodromal interval is likely to be critical and may even be sufficient to allow a cure.

At the same time, the memory capacity of the master control microprocessor will be sufficient to accommodate enough of the information contained in the Prescribers' Digital Reference for the system to specify a drug other than those stored, and in so doing, indicate a condition that had not been otherwise anticipated. If the drug has a different target, then depending upon the patient as mentioned, the recommendation presupposes the placement of a line for its delivery. Provided the system has been furnished with the requisite information, the system, at least when interpreted by an experience diagnostician, can also indicate the need for a surgical procedure.

As delineated in copending application Ser. No. 16/873,914, entitled Vascular Valves and Servovalves—and Prosthetic Disorder Response Systems, such a system can include or have added to its program a subroutine that would render it capable of administering a solid organ transplantation using the metered compound bypass technique. Features provided by a conventional electronic prescription, to include a decision support system, drug utilization safety review, warnings, and the identification of pertinent diagnostic tests not yet applied and any lower cost alternative drugs would be plainly advantageous if not novel.

It is novel, however, that the constant monitoring by the relevant sensor or sensors would instantly pass data up the system to the master control microprocessor signaling the need to instantly stop the release of if not counteract a drug causing unacceptable side effects. Such implies the use of sensors that would detect the side effect or effects and the counteractant if available had been included in the system as justified despite an overall approach advocating an otherwise conservative specification of components, drugs, and simplest of programs. Much the same rationale supports allowing the use of potentially problematic drugs and the use of radiation in the treatment of cancer.

Left connected and exiting the body when not in use as are lead-in catheters connected to intracorporeal vessels disallows vigorous activity and gradually increases irritation at the entry incision along with the risk of infection. In contrast, a totally implanted system incorporates a subcutaneously placed body surface port, when simple, a conventional portacath or mediport, one or more drug reservoirs, end-connectors, and implanted sensors without a line hanging outside the body for connection to extracorporeal equipment in the clinic. Communication with the implanted system is by means of a small—such as universal serial bus—plug inerted into a socket at the body surface or by wireless radio communication.

Conventional injection ports (portacaths, mediports, peripherally inserted central catheters) are connected to larger vessels, not at any level along the lumen of the immediate blood supply of a particular organ or volume of tissue to allow the targeting of a drug directly to deep within the parenchyma of that structure. For the treatment of chronic disease, safe and secure end-connectors for ductus such as vessels, ureters and the gut which will support a fully implanted prosthetic disorder response system for many years if not the life of the patient are described in copending application Ser. No 15/998,002 entitled Ductus Side-entry Jackets and Prosthetic Disorder Response Systems. Those for attachment to the surfaces of organs and tissues are described in U.S. Pat. No. 11,013,858, entitled Nonjacketing Side-entry Connectors and Prosthetic Disorder Response Systems.

As indicated, absent access to such end-connectors and therefore unable to target medication directly into the blood supply of a specific organ or delimited volume of tissue, dosing must be increased to compensate for dilution throughout the circulation while at the same time incur the risk and constraints imposed by the potential for adverse reactions in other organs and tissues. This inherently poses a ‘double edged sword’ of restricting the dose meant for the target as well as restricting the dose that might be allowed to circulate.

In contrast, release directly into the blood supply or the parenchyma of an organ through a leak-free and secure connector admits little of a drug or drugs in the general circulation and avoids release into the general circulation and the many problems associated with the exposure to the drug of nontargeted tissue, the adverse side effects this might arouse, and especially in comorbid disease requiring the administration of numerous drugs of which any or all might interact, adverse drug-drug and drug-food interactions. Drugs are positively beneficial in treating the tissue intended, but when not targeted, pose the risk of harm to other tissue. All drugs can cause side effects and adverse drug and food interactions. A few examples of common circumstances where pharmacy would be liberated by direct pipeline targeting include:

1. Drugs ordinarily contraindicated or dose-limited for a gravida which not constrained to the target but dispersed throughout the circulation pose a threat of harm to the fetus and post-delivery harm to the baby through lactation may be rendered usable. Were the drug directly pipelined to the lesion, in this case, malignant, or to the origin of the condition to be treated, the potential harm to the fetus caused by thalidomide, taken orally, with its several positive and effective uses, would be eliminated. Means for the prevention of entry into the general circulation of any residue not assimilated by the target are several and described in copending application Ser. No. 15/932,172, entitled Integrated System for the Infixion and Retrieval of Implants.

2. Prescribed to treat a mood, such as a bipolar disorder, often in the elderly, lithium poses the risk of hypothyroidism, or myxedema, “perhaps because lithium inhibits its hormone release by the thyroid” (Hershman, J. M. 2006. “Thyroid Disorders,” in The Merck Manual 18th edition, section 12, chapter 152, page 1201, Whitehouse Station, N.J.: Merck Research Laboratories).

Moreover, hypothyroidism can cause mental impairment and drowsiness. The clear implication is that preventing the exposure of the thyroid gland to lithium would eliminate would eliminate these sequelae. To the extent that lithium can cause these side effects indirectly by affecting the thyroid as opposed to directly by affecting the brain (Fawcett, J. 2006. “Mood Disorders,” in The Merck Manual 18th edition, section 15, chapter 200, page 1715), rather than to disperse lithium throughout the general circulation, it can be released directly into the internal carotids through ductus side-entry jackets.

Neither metabolized nor protein-bound, lithium is normally excreted by the kidneys, to which lithium is harmful. Accordingly, to avoid the thyroid and the kidneys following its direct passage into the brain through ductus side-entry jackets on the internal carotids requires a nonrenal route for its excretion.

As part of its ability to extract any magnetically susceptible carrier-bound molecule or particulate from the passing blood, the magnetic extraction system described in copending application Ser. No. 15/998,002 entitled Ductus Side-entry Jackets and Prosthetic Disorder Response Systems Ductus Side-entry Jackets, shown in FIGS. 39A and 39B, allows the direct delivery of a lithium salt to the brain through ductus side-entry jackets on the internal carotids, the corresponding drainage accomplished upstream from the larger inferior vena cava, this because drainage is initially nonunitary but rather divided among tributaries as to justify recovery from the inferior vena cava into which the tributaries had since emptied.

Once carried along in the inferior vena cava, the reverse process is used to draw the extract directly from the inferiors vena cava into the urinary bladder for expulsion through normal urination. When delivered directly to the brain with the rest of the circulatory system omitted, the absolute amount of lithium, or lithium salt, is relatively tiny to begin with. Moreover, this magnetically-based form of hemodialysis, implanted in the ambulatory patient, can function continuously, or to conserve power and extend the recharging interval, can be programmed to continue for an interval following the automatic release of the lithium. Either way, the lithium never sees either the thyroid nor the kidneys and cannot accumulate. For these reasons, supplementation with periodic conventional filtration-based hemodialysis (Hedya, S. A., Avula, A., and Swoboda, H. D. 2021. “Lithium Toxicity,” Treasure Island, Fla.: StatPearls Publishing, online) in the clinic is probably unnecessary.

3. Accordingly, extrarenal extraction eliminates the kidneys as means for extracting a substance injurious to them, as well as bypasses the thyroid gland, thus preventing hypothyroidism as well as nephrotoxicity along with the serious side effects of both. Additional benefits of magnetic extraction are the elimination of the need for a vascular access that will eventually become injured, and elimination of the process which commences with the insertion of a needle which for many patients is disturbing.

4. Chemotherapeutics, immunosuppressives, and steroids which can be directly pipeline-targeted to the site of a malignancy, for example, and if metastasized, then targeting the metastases as well, eliminates the many serious side effects caused by such medication. To destroy any ‘daughter’ cells shed by the primary tumor in the circulation, a smaller and less problematic background dose of the same or a different drug may destroy these, but if not, then one approach is to mark these using docking techniques as addressed below for direct destruction in the circulation or extraction through magnetically or filtration-based hemodialysis.

5. Anticholinergics and antimuscarinics which can be directly pipeline-targeted to an overactive bladder, keeping these well clear of the brain in elderly patients with a weakened blood brain barrier to prevent the induction or advancement of a dementia.

As indicated, conventional means of vascular access such as a central venous line or intravenous cannula are limited to temporary connection to the circulatory system, not to the blood supply of a certain organ, gland, or volume of tissue. At the same time, by secondarily targeting the circulatory system as a whole, a directly pipeline-targeted system can simultaneously apportion drugs for background systemic dispersal when ‘daughter’ cells shed by the primary tumor must be destroyed. Provided the disseminated dose can be lower than would ordinarily be required, the side effects associated with the dispersal of a chemotherapeutic would be reduced as well.

Connection with a ductus side-entry jacket allows the placement of a central line or conventional peripherally inserted central catheter for the diagnosis and/or treatment of a chronic condition through a small incision rather than blindly as when connecting to the subclavian vein, the ability to view the field eliminating the risk of a pneumothorax and most other complications. A direct view eliminates misplacement regardless of the cause such as when the patient is extremely obese or anatomically anomalous. Moreover, once securely fixed in position thus, the line can remain in place indefinitely with the patient fully ambulatory. One benefit of this can be compared to the constancy of a Holter monitor as opposed to an isolated electrocardiogram.

In conjunction with an implanted prosthetic disorder response system, this constancy is immediately served by remedial action. Such an implanted central line can serve for any purpose that central lines serve, to include use for vascular access for extracorporeal hemodialysis and plasma apheresis. Copending application Ser. No. 15/998,002, entitled Ductus Side-entry Jackets and Prosthetic Disorder Response Systems, additionally taught a magnetically based totally implanted plasma processing mechanism that extracted magnetically tagged analysts and conveyed these into the urinary bladder by reversed extraction.

Ultrasound guidance allows direct viewing during connection to the internal jugular thus reducing but not eliminating the risk of a pneumothorax or another, even more serious risk, which is cannulation of the common carotid with profuse internal bleeding. Once placed, the skin is closed with no line exiting the chest to invite infection through the break in the skin that demands the frequent application of an antiseptic to and changing of a protective dressing. To this extent, a peripherally inserted central catheter offers closed skin placement as well. Other complications eliminated include occlusion of the line as the result of an accumulation of thrombus due to blood reflux, this risk readily dispelled through the periodic release through the jacket-venous junction of a heparin or fondaparinux through the jacket service or accessory channel, or if this had been neglected so that clot had formed, a thrombolytic such as streptokinase.

Other potential substances that can occlude are similarly prevented, precipitates with highly dilute hydrochloric acid and tacky lipids with ethanol. Entry of a venous line using a ductus side-entry jacket is far safer than conventional insertion. Barring an accidental collision of the operator by another worker of apparatus, the risk of air embolism is essentially reduced to zero. This because the entry opening trepan cuts its way surrounded by jet streams of water directed toward the breech, closing off any surrounding gas and preventing extravasation. The process of ductus opening is described in copending application Ser. No. 15/998,002, entitled Ductus Side-entry Jackets and Prosthetic Disorder Response Systems.

Not directly targeted into the blood supply of an organ or volume of tissue to be treated, drugs are administered in doses adequate to be diluted and dispersed throughout the total volume of blood in the circulatory system but still in a strength adequate to affect the target. Without segregation from other tissue and drugs, adverse drug-drug and drug-food interactions and side effects go unconstrained. When a new and costly drug is needed, this high level dosing due to the inability of conventional administration to restrict delivery to a particular target creates such expense that a new and critically superior drug may need to be discounted. Reciprocally, the dose at the target must be limited to the concentration allowable for dispersion throughout the circulatory system.

This key deficiency of conventional drug administration not only violates the cardinal object of responsible health care in using the fewest drugs in the smallest doses possible but effectively liberates the excess to create complications elsewhere in the body. Not only does an indwelling catheter, central, or intravenous line, or peripherally inserted central catheter not allow fully implanted connection to other than major vessels or to the blood supply of smaller organs, glands, or tissues with the patient unrestricted in freedom of movement independent of the clinic, but were the patient accidentally struck, these risk serious complications of perforation and dislodgement.

Moreover, absent such an incident, the entry wounds at the body surface and at the vessel or tissue, while minor injuries at the outset, were the line left in place over a longer term, would demand frequent treatment with an antiseptic and redressing, laxity in this regard resulting in a gradual increase in injury and the likelihood of infection. These means are therefore limited to temporary use in the clinic where these connections can be monitored and maintained as necessary. Such a fully implanted ambulatory and automatic prosthetic disorder response system can be configured with sensors to diagnose and means for medicinal storage and release to treat any disease or disorder, and can be programmed to optimize this treatment.

That in a fully ambulatory patient, segregation of drug delivery in much greater proportion than drugs in the circulation to the originating site or sites of any chronic disorder or disease with minimal entry of drugs in the general circulation as could cause adverse interactions or side effects is medically significant. This, moreover, by means of a totally implanted system which can remain in place indefinitely to continuously diagnose and treat a congenital or chronic disorder—for years if not to the end of life, as plainly a material improvement in the quality of that life.

In response to sensor feedback indicative of the effect of the immediate drug doses and/or alternative such as electrostimulatory therapy, an automatic diagnostic and therapeutic system as addressed herein can continuously adjust both the identity and the dosing of the drugs, whether coordinated with electrostimulatory therapy to find the combination thereof which both most effective and least problematic.

This versatility is itself a central aspect of the directly pipeline-targeted concept of drug delivery. Even conventional circulatory assist devices intended to serve as implants on a long term basis—ventricular assist devices and artificial hearts, for example—fail to provide normal circulation and/or allow increasing injury, demanding frequent reexamination, and while often useful as life-saving bridges pending organ transplantation, are not adequate as end treatments.

Despite numerous attempts to produce an artificial heart that lasts for years, such is rare. The type implant system described here is fundamentally and critically distinct from such time-limited means in administering a heart transplant by a metered compound bypass technique and thereafter exercising vigilance over the function of the heart and instantly providing it with medication should the need arise. The technology to allow this is addressed in copending application Ser. No. 16/873,914, entitled Vascular Valves and Servovalves—and Prosthetic Disorder Response Systems.

This immediacy of targeted dosing responsive to feedback represents a speed and level of control not provided by nonautomated and nonpiped techniques of targeting. Such include inherently docking or the targeting of malignant cells on the basis of intrinsic chemistry through the use of a tumor-homing peptide to achieve enhanced tumor penetration, addressed shortly below. Also included are the incorporation into a tumor of magnetically susceptible matter, for example, and the use of optogenetic means, addressed in copending application Ser. No. 16/873,914, entitled Vascular Valves and Servovalves—and Prosthetic Disorder Response Systems. These nonpipe targeted priming, or docking, methods of attracting a curative agent are able to seek out like-kind malignant ‘daughter’ cells in the circulation, for example, but if not previously established on the basis of experience, to target multiple chemically distinct targets thus might require preparation requiring more time than a severely impaired or elderly and multiply comorbid patient would survive.

Whereas pipeline targeting can simultaneously segregate any kind and number of lesions, site, organ, or tissue, phage homing is substantially limited to seeking out a tissue such as a tumor based upon its unique intrinsic properties. The object in docking is to render a tumor less resistant to penetration by a drug used to treat the tumor (see, for example, Sugahara, K. N., Teesalu, T., Karmali, P. P., Kotamraju, V. R., Agemy, L., and 4 others 2009. “Tissue-penetrating Delivery of Compounds and Nanoparticles into Tumors,” Cancer Cell (Cambridge, Mass.) 16(6):510-520). Unrestricted to tumor chemistry, direct pipeline targeting is applicable to any type lesion and can be instituted in little time without the need for preliminary chemical research. Piping avoids this problem of differential selectability through simultaneously administered means to target different agents to different type targets. Such an approach allows targeting with relatively little lead time.

Significantly, whereas a pipeline-targeted system can avoid the general circulation to a significant if not exclusive extent, an uptake-increasing approach targets a tumor, for example, only once the medicinal has been dispersed in the general circulation to begin with. This has the advantage of systemic dispersal upon administration but is no less subject to the risk of side effects and adverse interactions, most if not all of which should be identified prior to testing in a clinical trial. By comparison, a piping system to treat a multiply comorbid patient with a variety of drugs and/or other agents to treat different diseases and different lesions is emplaced in a single procedure taking relatively little preparatory time compared to that required to use such an alternative approach.

Not all disease is comorbid or otherwise untenably complicated, and advancements in the homing approach are appearing that make such technology potentially advantageous not just in itself but with much potential when used in combination with direct pipeline targeting. Different type targets necessitate differently prepared biomimetic nanoparticulate coatings both to tag and penetrate both tumors and other type lesions. However, tumor-homing peptides can reveal metastases to imaging and therefore serve to speed up the process of clarifying the routing of targeting pipelines to deliver chemotherapeutics, for example, using different means for destroying abscopal metastases amid a background of other type lesions.

Clearly, the ability to simultaneously target numerous distinctly different lesions without regard to target or targeting chemistry is fundamentally less complicated and less subject to problems than is a biomimetic approach. Significantly, targeting by this or any other chemically based method still disperses the agent throughout the circulatory system, posing the potential for drug-drug interactions and adverse side effects. In fundamental contrast, each pipe-targeted drug can only cause side effects and drug interactions that involve the target but not other tissue. Direct pipeline targeting into the blood supply of the target also makes it possible for the drug targeted to be disproportionately, unlimitedly larger in dose compared to the drugs that enter the blood supply through the general circulation.

Simplicity and minimizing the cost for an effective implanted prosthetic disorder response system important objectives, only drugs that must or would much benefit from isolated delivery are pipeline-targeted. While drugs generally achieve more thorough penetration when delivered through the blood supply, drugs and other therapeutic agents in liquid form can be directly pipeline-targeted at or into an intracranial tumor extravacularly (glioma, glioblastoma multiforme, ependymoma, or medulloblastoma (see, for example, Shapiro, W. R. 2006. “Intracranial and Spinal Tumors,” The Merck Manual, Whitehouse Station, N.J.: Merck Research Laboratories, chapter 225, page 1916). Targeting other than through the blood supply is through a ultrasound probe and stereotactically guided direct-to-or-into the tumor pipeline physically passing through and accordingly, unobstructed by the blood brain barrier.

“The unsatisfactory therapeutic outcome for glioma is mainly due to the poor blood-brain barrier (BBB) permeability and inefficient accumulation in the glioma area of chemotherapeutic agents.” (Liang, J., Gao, C., Zhu, Y., Ling, C., Wang, Q., and 5 others 2018. “Natural Brain Penetration Enhancer-modified Albumin Nanoparticles for Glioma Targeting Delivery,” American Chemical Society Applied Material and Interfaces 10(36):30201-30213). This assertion makes it clear that treatment immediately upon detection of the malignancy which passes the blood brain barrier should yield improved outcomes.

Because separate forms of delivery may offer an advantage with different combinations of drugs that would best come into contact to first react with one another only once inside the tumor, it is possible to use the extravascular piped route for one component and the vascular route for the other at the same time. The homing nanoparticle approach appears capable of overcoming the barrier as well, and new vascular methods for passing through the barrier without mannitol or piping are under development, made the more significant because any fluid medicinal can be targeted to the brain through a ductus side-entry jacket on one or both internal carotids, much reducing the dose compared to a dose large enough for dilution throughout the circulatory system.

Alternatively then, a nanoparticulate coating of various therapeutic agents can be devised to be drawn to and into a brain tumor through the blood supply (see, for example, Song, J., Lu, C., Leszek, J., and Zhang, J. 2021. “Design and Development of Nanomaterial-based Drug Carriers to Overcome the Blood-Brain Barrier by Using Different Transport Mechanisms,” International Journal of Molecular Sciences 22(18):10118; Thangudu, S., Cheng, F. Y., and Su, C. H. 2020 “Advancements in the Blood-Brain Barrier Penetrating Nanoplatforms for Brain Related Disease Diagnostics and Therapeutic Applications,” Polymers (Basel, Switzerland) 12(12):3055; Lundy, D. J., Lee, K.-J., Peng, I.-C., Hsu, C.-H., Lin, J.-H., and 3 others 2019. “Inducing a Transient Increase in Blood-Brain barrier Permeability for Improved Liposomal Drug Therapy of Glioblastoma Multiforme,” American Chemical Society Nano 13(1):97-113; Kang, S., Shin, W., Choi, M.-H., Ahn, M., Kim, Y.-K., and 3 others 2018. “Morphology-controlled Synthesis of Rhodium Nanoparticles for Cancer Phototherapy,” American Chemical Society Nano 12(7):6997-7008; Liang, J., Gao, C., Zhu, Y., Ling, C., Wang, Q., and 5 others 2018, Op cit.; Teleanu, D. M., Chircov, C., Grumezescu, A. M., Volceanov, A., and Teleanu, R. I. 2018. “Blood-Brain Delivery Methods Using Nanotechnology,” Pharmaceutics (Basel, Switzerland) 10(4):269; Lin, T., Zhao, P., Jiang, Y., Tang, Y., Jin, H., and 4 others 2016. “Blood-Brain-barrier-penetrating Albumin Nanoparticles for Biomimetic Drug Delivery via Albumin-binding Protein Pathways for Antiglioma Therapy,” American Chemical Society Nano 10(11):9999-10012; Barker, H. E., Paget, J. T. E., Khan, A. A., and Harrington, K. J. 2015. “The Tumour Microenvironment after Radiotherapy: Mechanisms of Resistance and Recurrence,” Nature Reviews. Cancer 15(7):409-425).

“Docking-based (synaphic) targeting strategies use peptides, antibodies and other molecules that bind to tumor vessels and tumor cells to deliver more drug to tumors than to normal tissues.” Sugahara, K. N., Teesalu, T., Karmali, P. P., Kotamraju, V. R., Agemy, L., and 4 others 2009, Op cit.; see also, for example, Beh, C. Y., Prajnamitra, R. P., Chen, L.-L., and Hsieh, P. C.-H. 2021. “Advances in Biomimetic Nanoparticles for Targeted Cancer Therapy and Diagnosis,” Molecules 26(16):5052; Chen, C., Song, M., Du, Y., Yu, Y., Li C., and 4 others 2021. “Tumor-associated-macrophage-membrane-coated Nanoparticles for Improved Photodynamic Immunotherapy Nano Letters 21(13):5522-5531; Chen, L., Hong, W., Ren, W., Xu, T., Qian, Z., and He, Z. 2021. “Recent Progress in Targeted Delivery Vectors Based on Biomimetic Nanoparticles,” Signal Transduction and Targeted Therapy 6(1):225; Huang, S.-S., Lee, K.-J., Chen, H.-C., Prajnamitra, R. P., Hsu, C.-H., and 15 others 2021. “Immune Cell Shuttle for Precise Delivery of Nanotherapeutics for Heart Disease and Cancer,” Science Advances 7(17)eabf2400; Iqbal H., Yang T., Li T., Zhang M., Ke H., and 3 others 2021. “Serum Protein-based Nanoparticles for Cancer Diagnosis and Treatment,” Journal of Controlled Release 329:997-1022; Jiang X., Stockwell, B. R., and Conrad, M. 2021. “Ferroptosis: Mechanisms, Biology and Role in Disease,” Nature Reviews. Molecular Cell Biology 22(4):266-282; Mahmoudpour, M., Ding, S., Lyu, Z., Ebrahimi, G., Du, D., and 3 others 2021. “Aptamer Functionalized Nanomaterials for Biomedical Applications: Recent Advances and New Horizons,” Nanotoday 39:101177; Pediconi, N., Ghirga, F., Del Plato, C., Peruzzi, G., Athanassopoulos, C. M., and 10 others 2021. “Design and Synthesis of Piperazine-based Compounds Conjugated to Humanized Ferritin as Delivery System of siRNA in Cancer Cells,” Bioconjugate Chemistry 32(6):1105-1116; Wang, J., Li, Y., and Nie, G. 2021. “Multifunctional Biomolecule Nanostructures for Cancer Therapy,” Nature Reviews. Materials May 19; 1-18; Zhao, Q., Gong, Z., Li, Z., Wang, J., Zhang, J., and 6 others 2021. “Target Reprogramming Lysosomes of CD8+ T Cells by a Mineralized Metal-Organic Framework for Cancer Immunotherapy,” Advanced Materials (Deerfield Beach, Fla.) 33(17):e2100616; Cai, Y., Wang, Y., Zhang, T., and Pan, Y. 2020. “Gadolinium-labeled Ferritin Nanoparticles as Ti [spin-lattice relaxation time constant] Contrast Agents for Magnetic Resonance Imaging of Tumors,” American Chemical Society Applied. Nano Materials 3(9):8771-8783; Cheng, X., Fan, K., Wang, L., Ying, X., Sanders, A. J., and 11 others 2020. “TfR1 [binding transferrin receptor 1] Binding with H-ferritin Nanocarrier Achieves Prognostic Diagnosis and Enhances the Therapeutic Efficacy in Clinical Gastric Cancer,” Cell Death and Disease 11(2):92; Gan, J., Du, G., He, C., Jiang, M., Mou, X., Xue, J., and Sun, X. 2020. “Tumor Cell Membrane Enveloped Aluminum Phosphate Nanoparticles for Enhanced Cancer Vaccination,” Journal of Controlled Release 326:297-309; Gong, C., Yu, X., You, B., Wu, Y., Wang, R., and 4 others 2020. “Macrophage-cancer Hybrid Membrane-coated Nanoparticles for Targeting Lung Metastasis in Breast Cancer Therapy,” Journal of Nanobiotechnology 18:92; Ma, J., Liu, F., Sheu, W. C., Meng, Z., Xie, Y., and 18 others 2020. “Copresentation of Tumor Antigens and Costimulatory Molecules via Biomimetic Nanoparticles for Effective Cancer Immunotherapy,” Nano Letters 20(6):4084-4094; Sindhwani, S., Syed, A. M., Ngai, J., Kingston, B. R., Maiorino, L., and 15 others 2020. “The Entry of Nanoparticles into Solid Tumours,” Nature Materials 19(5):566-575; Xu, N., Yang, Y.-F., Chen, L., and Lin, J. 2020. “A Ferritin-Albumin-Cu Nanoparticle that Efficaciously Delivers Copper(II) Ions to a Tumor and Improves the Therapeutic Efficacy of Disulfiram,” American Chemical Society Omega 5(18):10415-10422; Yang, Z., Ma, Y., Zhao, H., Yuan, Y., and Kim, B. Y. S. 2020 “Nanotechnology Platforms for Cancer Immunotherapy, Wiley Interdisciplinary Reviews. Nanomedicine and Nanobiotechnology 12(2):e1590; Edwardson, T. G. W. and Hilvert D. 2019. “Virus-inspired Function in Engineered Protein Cages,” Journal of the American Chemical Society 141(24):9432-9443; He, J., Fan, K., and Yan, X. 2019. “Ferritin Drug Carrier (FDC) for Tumor Targeting Therapy,” Journal of Controlled Release 311-312:288-300; Park, J., Choi, Y., Chang, H., Um, W., Ryu, J. H., and Kwon, I. C. 2019. “Alliance with EPR [enhanced permeability and retention] Effect: Combined Strategies to Improve the EPR Effect in the Tumor Microenvironment,” Theranostics 9(26):8073-8090; Wang, H., Wu, J., Williams, G. R., Fan, Q., Niu S., and 3 others 2019. “Platelet-membrane-biomimetic Nanoparticles for Targeted Antitumor Drug Delivery,” Journal of Nanobiotechnology 17:60; Patra, J. K., Das, G., Fraceto, L. F., Campos, E. V. R., del Pilar Rodriguez-Torres, M., and 7 others 2018. “Nano Based Drug Delivery Systems: Recent Developments and Future Prospects, Journal of Nanobiotechnology 16(1):71; Senapati, S., Mahanta, A. K., Kumar, S., and Maiti, P. 2018. “Controlled Drug Delivery Vehicles for Cancer Treatment and Their Performance,” Signal Transduction and Targeted Therapy 3:7; Shan, W., Zhang, D., Wu, Y., Lv, X., Hu, B., and 5 others 2018 “Modularized Peptides Modified HBc [hepatitis B core protein] Virus-like Particles for Encapsulation and Tumor-targeted Delivery of Doxorubicin,” Nanomedicine: Nanotechnology, Biology, and Medicine 14(3):725-734; Weiβ, E. and Kretschmer, D. 2018. “Formyl-peptide Receptors in Infection, Inflammation, and Cancer,” Trends in Immunology 39(10):815-829; Zhang, Y., Cai, K., Li, C., Guo, Q., Chen, Q., and 9 others 2018. “Macrophage Membrane-coated Nanoparticles for Tumor-targeted Chemotherapy,” American Chemical Society Nano Letters 18(3):1908-1915; Bhaskar, S. and Lim, S. 2017. “Engineering Protein Nanocages as Carriers for Biomedical Applications,” NPG [Nature Publishing Group] Asia Materials 9(4):e371; Leng, Q., Woodle, M. C., and Mixon, A. J. 2017. “Targeted Delivery of siRMA Therapeutics to Malignant Tumors,” Journal of Drug Delivery 2017: 6971297; Li, J. and Mooney, D. J. 2016. “Designing Hydrogels for Controlled Drug Delivery,” Nature Reviews. Materials 1(12):1607; Ruoslahti, E. 2017. “Tumor Penetrating Peptides for Improved Drug Delivery,” Advanced Drug Delivery Reviews 110-111:3-12; Bhatia, S. 2016. “Nanoparticles Types, Classification, Characterization, Fabrication Methods and Drug Delivery Applications,” in Natural Polymer Drug Delivery Systems, Cham, Switzerland: Springer International, pp. 33-93; Ramalho, J., Semelka, R. C., Ramalho, M., Nunes, R. H., AlObaidy, M., and Castillo, M. 2016. “Gadolinium-based Contrast Agent Accumulation and Toxicity: An Update,” American Journal of Neuroradiology 37(7):1192-1198; Wilhelm, S., Tavares, A. J., Dai, Q., Ohta, S., Audet, J., Dvorak, H. F., and Chan, W. C. W. 2016. “Analysis of Nanoparticle Delivery to Tumours,” Nature Reviews. Materials 1:16014; Christianson, H. C., Svensson, K. J., van Kuppevelt, T. H., Li, J.-P., and Belting, M. 2013. “Cancer Cell Exosomes Depend on Heparan Sulfate Proteoglycans for Their Internalization and Functional Activity,” Proceedings of the National Academy of Sciences of the United States of America 110(43): 17380-17385; Prabhakar, U., Maeda, H., Jain, R. K., Sevick-Muraca, E. M., Zamboni, W., and 5 others 2013. “Challenges and Key Considerations of the Enhanced Permeability and Retention Effect for Nanomedicine Drug Delivery in Oncology” Cancer Research 73(8):2412-2417; Maeda, H. 2010. “Tumor-selective Delivery of Macromolecular Drugs via the EPR [enhanced permeability and retention] Effect: Background and Future Prospects,” Bioconjugate Chemistry 21(5):797-802; Park, J.-H., von Maltzahn, G., Zhang, L., Derfus, A. M., Simberg, D., and 4 others 2009. “Systematic Surface Engineering of Magnetic Nanoworms for in Vivo Tumor Targeting,” Small (Weinheim an der Bergstrasse, Germany) 5(6):694-700; Murphy, E. A., Majeti, B. K., Barnes, L. A., Makale, M., Weis, S. M., and 3 others 2008. “Nanoparticle-mediated Drug Delivery to Tumor Vasculature Suppresses Metastasis,” Proceedings of the National Academy of Sciences of the United States of America 105(27):9343-9348; Park, J.-H., von Maltzahn, G., Zhang, L., Schwartz, M. P., Ruoslahti, E., Bhatia, S. N., and Sailor, M. J. 2008. “Magnetic Iron Oxide Nanoworms for Tumor Targeting and Imaging,” Advanced. Materials (Deerfield Beach, Fla.) 20(9):1630-1635; Simberg D, Duza T, Park J H, Essler M, Pilch J, and 7 others 2007. “Biomimetic Amplification of Nanoparticle Homing to Tumors,” Proceedings of the National Academy of Sciences of the United States of America 104(3):932-936; Cen, D., Brayton, D., Shahandeh, B., Meyskens, F. L., and Farmer, P. J. 2004. “Disulfiram Facilitates Intracellular Cu Uptake and Induces Apoptosis in Human Melanoma Cells,” Journal of Medicinal Chemistry 47(27):6914-6920; Porkka, K., Laakkonen, P., Hoffman, J. A., Bernasconi, M., and Ruoslahti E. 2002. “A Fragment of the HMGN2 [human high mobility group protein 2] Protein Homes to the Nuclei of Tumor Cells and Tumor Endothelial Cells in Vivo,” Proceedings of the National Academy of Sciences of the United States of America 99(11):7444-7449).

Entry wounds at the body surface and at the entry into ductus, or tubular anatomical structures, and tissue are devised to be small, unobtrusive, stable, leak-free, capped over to prevent microbial intrusion, and easily sterilized by wetting with a liquid antiseptic. When unavoidable for recurrent flow of urine or the passage of miniature cabled diagnostic and therapeutic devices, or to pass electrical conductors or fluid pipelines when the size, weight, or number of these exceed the number that can be implanted, for example, body surface wounds not fully closed at the time of system placement are provided with a protective surface port that securely closes off the entry. Intracorporeally unaccommodable components are then relegated to a body pack worm about the waist.

As with entry through a portacath, or mediport, the injection or infusion of drugs and other agents is into a body surface port positioned subcutaneously, or subdermally, the skin overlying the entry wound allowed to heal by first intention. However, the distal terminus of any such line is not an indwelling catheter but rather a connector designed to remain secure on a permanent basis and not interfere with the ability of the patient to engage in work or most outdoor activities. Both periductal drug feeding and blood or urine takeoff jackets and organ and flat tissue surface connectors are devised to prevent extravasation upon placement and are provided with openings and padding to protect the nervelets and small vessels at the substrate surface.

Where contact occurs, the cushioned lining is made of highly compliant foam, if necessary, vapor deposited, or sputter coated, with a nanometric coating of parylene specially copolymerized, plasticized, and if necessary, annealed, for high elasticity, and embedded with an antimicrobial placed at the foam-ductus contact interface as well as the application of a nanometric coating of parylene elsewhere to prevent breakdown due to hydrolytic and/or catalytic action, thus preserving the ability of the foam to conform to small structures at the adventitia or fibrosa by gentle investment rather than by compression (see, for example, Galeotti, F., Andicsova, A., Bertini, F., Laux, E., Hartmann, L., and 6 others 2014. “Enhanced Elasticity in Parylene Thin Films by Copolimerization Approach,” Journal of Materials Science 49(21):7547-7555; Lendlein, A. and Langer, R. 2002. “Biodegradable, Elastic Shape-memory Polymers for Potential Biomedical Applications,” Science 296(5573):1673-1676).

No less significant is the configurability of the system to diagnose and treat any problem of internal medicine using any drugs and/or other therapeutic means such as electrostimulatory, thermal, or these in a coordinated manner. To this must be added the constant entry into the market of new drugs and treatment modalities, each often having adjuvant or supportive benefit when used in combination with other drugs, and often, off-label applications. For these reasons, any description of such system less than encyclopedic in length best delineates system application in a circumscribed area, here, urological, with the understanding that such a system is adaptable to any other condition.

As delineated in copending application Ser. No. 16/873,914, entitled Vascular Valves and Servovalves—and Prosthetic Disorder Response Systems, such a system can not only optimize treatment but can administer and optimize the progress of surgical procedures to include solid organ transplantation and the replacement of extensive lengths of major vessels that ordinarily require the experience and skill of specialists at large centers in developed countries, bringing these procedures within the purview of general surgeons worldwide. In replacing a segment along a major vessel, a fabric with an expandability and strength that makes it possible for vessels implanted in a very young patient to expand with growth can eliminate the revisions that would further debilitate an unwell child.

The urological application of a prosthetic disorder response system to support the drainage system shown in FIG. 1 is strictly exemplary: any disorder or disease the subject of internal medicine might have been cited, the number of possible combinations thereof vast. Any such system comprises a centralized set of drug reception, drug and power storage, and control components which common to many applications, preside over a subsidiary set of pipelines, connectors, and valves of which the distribution responds to the specific diagnosis.

FIG. 1 has been vertically contracted to bring the former set of components—usually positioned in the pectoral region—closer to the latter for compactness, and the urine outlet port shown positioned epicutaneously on the right side of the patient is actually placed to a side of the mons pubis. Except where the application is simple, such as that shown in FIG. 3 so that nothing more than a drugline with service or accessory channel has been placed to target a coronary artery for the direct release of a platelet blocker and/or thrombolytic and drug replenishment is manual by injection into the body surface port without the need for automatic control, the former set of components is omitted.

The nonsubcutaneously positioned external body port shown in FIG. 3 indicates that over time, additional diagnostic and therapeutic components such as cabled devices are expected to be needed, disallowing a subcutaneous position without the additional openings or sockets that will be needed. A subcutaneously placed port can incorporate multiple openings leading to different targets. When subcutaneous, a tiny tattoo on the skin—lased away if and when the system can be removed—indicates the point for insertion of each hypodermic needle. The prepositioning of anticipated components allows a system to be introduced in stages so that the patient allowed an interval for rest before any more need be added.

When transcutaneous energy transfer is undesired, the surface port provides an electrical receptacle to plug in a power cord. Practically speaking, however, the value in such a drugline is realized when drug release is automatically controlled according to a prescription-program executed by a digital controller. In relatively simple disease, control is by a microcontroller, whereas in more complex comorbid disease control is by a microprocessor. In FIG. 1, part number 53 is the system microcontroller, or in comorbid disease, the system microprocessor.

Updating the Prescription-Program

The production of prescription-programs demands not only expertise in computer programming such as Forth open firmware for its compactness, but an extensive and detailed practical clinical ‘hands-on,’ as well as ‘book’ knowledge, of internal medicine, pharmacy, pharmacology, and biostatistics. For this reason, even what might appear a relatively simple problem—to design an automatic disorder response system to coordinate the delivery of continuously monitored therapy adjusted as necessary with an awareness of the various secondary symptoms likely to appear later and then to require extended surveillance and the provision of remedial means for a patient with no more than two comorbidities from the outset. The automatic response system able to release ameliorative drugs directly into the internal carotids, comorbidity in this context comprehends psychological as well as physiological maladies of which the neuropsychiatric distinction may be unclear.

In the case of common comorbidities such as heart failure and diabetes, a single highly expert and experienced clinician may have acquired the detailed knowledge to anticipate the full complement of potential sequelary symptoms, some remote in terms both of distance from the primary site of disease in the body as well as in the interval preceding the emergence of secondary symptoms whether dependent or independent. Most often it will be essential to convene a prescription-program production panel in order to anticipate the directions an initially singular, much less comorbid disease will take so that the initial placement of components will prove sufficient without the need for reentry in order to place additional components at a later date.

To the extent possible, symptoms are anticipated and planned for without attribution to a specific morbidity or syndrome such as pleiotropic, or consequential or secondary. Should it eventuate that nonessential components had been placed, these will have been devised for permanent placement, eliminating the need for their removal. It is also possible, that the need for these remained inapparent for a long time postprocedurally when the need therefor became evident.

When morbidities tend to be affiliated, this should be taken into account as the need to reenter at a later date, much less more than once in order to add sensors and druglines as necessary is to be avoided. The larger the number of expert specialists that participate in the prescription-program production process, the less will be the odds for a need for reentry at a later date. For speed and economy, whenever the course in disease can be dependably predicted, prescription-programs should be standardized, referred for legal approval, and packaged with software, hardware, and instructions included.

The less familiar the morbidity or comorbidity, the wider the range of specialists that should participate in the process of prescription program production. When the need to monitor certain organs, glands, or tissues for symptoms cannot be predicted, suspect sites have sensors placed so that the system itself will reveal the need to treat those unanticipated. Then reentry is avoided by prescribing oral medication, for example. If this does not provide adequate therapy, the patient is best reentered to augment the existing prescription-program by adding an axis to the hierarchical control system and implant the necessary hardware to support this axis.

Updating the prescription-program may be routine, such as upon the approval of a new and more effective drug, of importance but less than critical, or exigent, as when the clinic receives sensor data indicative of an emergency condition. Provided transmission is restricted to a Internet protocol address or unique identifier, which is to say encrypted for any but the digital controller implanted in the patient intended, methods of Internet-implemented data transmission and reception applicable to communication between a remote prescription-programmer in the clinic and that controller include remote computer access or remote desktop protocol, over the air programming, Internet of things networking, and comparable technology as may appear.

To allow minor edits such as to correct typographical errors during an office visit to replenish the implanted drug reservoirs, for example, an above-skin rather than a subcutaneous body surface port can incorporate a standard telephone modular jack or a universal serial bus type socket for example, for insertion of a modular connector or USB plug led from the prescription-programmer's keyboard to the system digital controller, usually a microprocessor. Information concerning the health status of an identifiable patient must be receivable in intelligible form only by the control system implanted in the patient as data subject.

More important and sensitive changes such as the addition of a new subroutine in response to change in the condition of the patient which extensive enough to be tedious for both patient and programmer are best prerecorded and relegated to a universal serial bus, or ‘jump drive’ for entry in the clinic or by a local practitioner. Readily foreseeable adverse conditions are provided for in the original prescription-program. Emergency adjustments seek to make use of the agents on hand in the implanted drug reservoirs, and if the clinic is out of reach, the patient is advised to go to the closest druggist where his clinician has transmitted a prescription along with instructions for injecting the drug or drugs into implanted reservoirs.

Signaling by the implanted master control microprocessor of a need for responsive action due to an urgent condition not anticipated by or requiring revision in the existing prescription-program demands immediate response. Such a condition is detected as a rapidly progressing or cascading process such as the earliest signs indicative of an incipient myocardial or cerebral infarction signaled by sensors of symptoms affected by if not associated with the disease process for which the system as configured lacks the wherewithal to respond. Ideally accomplished while the patient remains ambulatory and unaware of it, any such emergent condition is automatically transmitted by an implanted medical telemetry transmitter, for example to the clinic.

Sensors almost certain to be included in any system that would detect an incipient myocardial or cerebral infarction, for example, are addressed in copending application Ser. No. 16/873,914, filed on 11 Aug. 2020, entitled Vascular Valves and Servovalves—and Prosthetic Disorder Response Systems. In this regard, even if not effectuated, when various contingencies would appear to be more consistent with the general condition of a patient, system circuitry and components can be prepositioned to ‘lie in wait’ and be activated to respond to such an event.

For emergency use, drugs in addition to those specified for the prescribed load list are provided in one or more standby drug reservoirs, turreted only when the number of contingencies justifies. The signaling of an urgent need for responsive action outside the purview of the existing program or requiring revision of the program demands immediate response. Such a condition is made known on the basis of a rapidly progressing or cascading process signaled by disease-related sensors of analytes associated with the disease process implanted in the patient. Such sensors are addressed in copending application Ser. No. 16/873,914 entitled Vascular Valves and Servovalves—and Prosthetic Disorder Response Systems.

Emergency response with the aid of remote electronic means is similar to the automatic updates to personal computer programs but targeted to the address specific to the digital controller implanted in the patient (see, for example, Schulz, C., Raff, S., Kortmann, S., and Obwegeser, N. 2021. “Digital Age Organizations: Uncovering Over-the-Air Updates in the Smart Product Realm,” 2021 International Conference on Information Systems online at aisel.aisnet.org/icis2021/iot_smart/iot_smart/1/; Gascón, D., Bielsa, A., Genicio, F., and Yarza, M. 2011. “Over the Air Programming with 802.15.4 and ZigBee—OTA—Laying the groundwork, online atlibelium.com/wp-content/uploads/2013/02/over_the_air_programming pdf).

FIG. 6 distinguishes between implanted components from those which due to their size and/or number had to be relegated to a body pack. Constituted to treat mono- or comorbid disease, such a system is negative feedback driven, sensor feedback applied continuously as remedial action is taken to continuously indicate the instant proximity to and the realization of having arrived at the set points indicative of normalcy. universal serial bus

Quite apart from serving as but one element in a comprehensive automatic comorbidity response system under the control of an implanted microprocessor, no more than a nonjacketing side-entry connector, part number 61 in FIG. 1, positioned as shown, gravity fed from a commercial portacath, or mediport 46, typically positioned subcutaneously in the pectoral area, the outlet thereof releasing a liquid antiseptic such as benzalkonium, hydrogen peroxide, or if preceded with a topical anesthetic or the patient is tolerant of a stinging sensation, an alcohol or highly dilute solution of sodium hypochlorite, povidone-iodine, chlorhexidine gluconate, or hexachlorophene would avert repeated lower urinary tract urinary infections as often affects women and the elderly.

Pathways into the urinary bladder other than through the urethra from the body surface port to and through nonjacketing side-entry connectors positioned as 61 and 62 allow an alternative approach passage or passages for cystoscopy when the bladder is infected, eliminating the interval imposed to proceed with diagnosis and other therapy until an antibiotic has eradicated a concurrent infection. Approach of a ureteroscope from above the level of infection, or antegrade, rather than through the infected area avoids the spreading of infection up into the kidneys.

For a condition that calls for repeated visual examination at intervals and not just a one or two instances thereof, a ductus side-entry jacket positioned high on a ureter accessed through a small body surface port with protective spring cap provides a pathway for the passage of a ureteroscope or intravascular ultrasound probe to examine the ureter and bladder without the risk of spreading infection to the kidneys. Access to the contralateral ureter is through another opening in the same surface port, not through a branched line as imposes the need to be viewed with ultrasound and is prone to be uncooperative in allowing easy entry into the passage wanted.

Similarly, the arrangement shown in FIG. 2 is intended for chronic medical as well as societal conditions much alleviated by the ability to switch the outflow of urine to bypass the lower urinary tract into a collection bag or urinal, usually cinched about a thigh. This ability not only allows voiding when inopportune such as wherever a bathroom is lacking, but eliminates the harrying of nocturia, urge incontinence, frequency, and dysuria. For a public speaker, athlete, or performer, for example—especially when afflicted thus—the need to void mid-event is eliminated, allowing wearers to avoid the need to abandon their chosen occupation.

No less important is the diagnostic advantage gained in the ability to pass the outflow of urine through or to bypass the lower tract. Any obstruction or stricture along the lower tract is bypassed as well. The examination of urine samples taken with the lower tract included and excluded allows an attribution of infection or constriction, for example, to the lower tract, clarifying which following method for imaging and treatment is most appropriate.

The advantage in clarifying the probable consequence of instilling medication into the bladder is clarified from the outset. At the same time, the absence of sensation from the lower tract when bypassed does nothing to detract from the ability to detect the use of drugs from an examination of the urine. The absence of sensation in a switchable as opposed to a prosthetic embodiment also makes it important to periodically switch to lower tract flow-through intentionally so that any pain arising from it will be sensed and referred for treatment.

Moreover, it warrants emphasis that unlike the use of a Foley or condom-type catheter leading to a collection bag, which also eliminate the need for a bathroom, here takeoff at the ureters bypasses the sensory nerve endings in the bladder and internal and external sphincters, eliminating any sensation of the constant outflow of urine, urge sensation, or the need to initiate micturition as a conscious action that would divert attention away from the ongoing public activity. Significantly, that a controllable embodiment allows the bladder to be bypassed as desired and a prosthetic embodiment bypasses the bladder permanently allows an undistorted view of the bladder interior in the absence of urine for diagnosis and treatment.

Inadequate emptying of the bladder often results in the accumulation of debris such as dead urothelial cells and leukocytes suspended in the urine that interfere with allowing a clear view of the bladder wall during cystoscopy. Unless there is a need for frequent reexamination and treatment, the latter, however, is not suggested as justification for placing the system Cystoscopic entry into the bladder from above is through a ductus side-entry jacket fastened about the ureter just beneath the pelvoureteral junction, or shortly below the renal pelvis no differently than as shown in relation to the left anterior descending coronary in FIG. 3.

Significantly, the creation of an approach from a superior position along the ureter, for example, allows the use of an antiseptic that will kill the pathogen whether bacterial, viral, fungal, or these in any combination. The importance of avoiding the spreading of a urinary tract infection is taken up below. Such a routine delay of weeks might allow the progression and detention of treatment of a severely degenerative process, to include the passage of uropathogenic bacteria, for example, up into the kidneys, causing a more serious condition such as pyelonephritis and/or urosepsis.

Moreover, if on the presumption that a common bacterium, Escherichia coli, which usually is the case, was the cause so that the pathogen had not been cultured and positively identified early in treatment, or more than one pathogen was responsible, yet more time will be lost following the prescription of an antibiotic or other antipathogen having little or no ability to cure the infection. Extraurethral access also allows the application or more diagnostic and therapeutic cabled apparatus to identify the cause of infection at the outset and not after the passage of weeks during which the patient continues dysuric. The highly flexible, shape compliant, and leak-free connectors can incorporate central openings sufficient in diameter to afford passage into the bladder of cabled or corded apparatus too large in diameter to be passed through the urethra.

In exceptional circumstances, the connectors can remain in place indefinitely with passage thereunto through an opening in the body surface port. This can serve to do away with the use of an antibiotic with its risks of overuse and the development of pathogen resistance and the adverse digestive consequences of overkilling the intestinal biota. Similarly, an anesthetic dripped through the same or a similar connector into the bladder can dispel the painful burning sensation in the urethra during voiding, especially with frequency. Providing a body surface port with a button the user can depress upon sensing the urge avoids the need for the periodic release of the anesthetic automatically.

A lower urinary tract infection as an isolated condition is treated with oral ciprofloxacin, sulfameth/trimethoprim, or nitrofurantoin, for example; however, if the patient, often elderly, has need of an implanted system for one or more other reasons or experiences reinfections of the urinary tract on a frequent basis, the addition to the system for this limited purpose, consisting of no more than a surface port, microcontroller, drug reservoir, pipeline, and one or two connectors, avoids the use of systemic medication, and functioning automatically, and does away with dependency upon regimen compliance, a common problem due to cognitive impairment in the elderly. Release into the bladder or through valves along the ureters of an antiseptic, if necessary, with an anesthetic, ameliorates if not eliminates the discomfort of infection before it kills the infection itself.

Access into the tissue of an organ is no less often accomplished by pipeline delivery of agents from the implanted drug reservoir through a ductus side-entry jacket along the organ arterial supply for passage into the lumen. In FIG. 1, release of the antiseptic is governed by a rudimentary control system consisting of a thin film strain gauge pressure sensor incorporated into connector 61 when the bladder fills so that the roof of the detrusor urinae commences to undulate or ripple as urge sensation begins. The control microcontroller and circuit are powered by a transcutaneously recharged button cell battery alongside portacath 46, which is replenished with the antiseptic when the symptoms of reinfection appear, to include a painful burning sensation of the urethra during urination, urinary incontinence, and frequent urination.

When the detrusor is weak so that the residual volume of urine in the bladder is excessive, a small automatically energized turbine shown and described in copending application Ser. No. 16/873,914, entitled Vascular Valves and Servovalves—and Prosthetic Disorder Response Systems, FIGS. 28 thru 30, allows thorough emptying. In copending application Ser. No. 16/873,914, the native bladder has been removed so that a prosthetic bladder is seen in its place.

Delivery into the bladder can be through any ureteral side-entry jacket or valve or through a nonjacketing side-entry connector mainline attached to the bladder roof or through an service or accessory channel, or sideline, of either type connector. Such a simple arrangement, consisting of no more than a portacath, catheter, and end-connector devised to remain implanted indefinitely can not be replaced by an indwelling catheter, which must not be permanently left in the body as in an accident or during rigorous exercise, it can result in a puncture or incisional injury through the substrate ductus and will gradually injure the tissue through which it had been passed.

A problem in ureteroscopy is the spreading of infection when advancing from an inferior level along the tract up toward the kidney. Access into the bladder commencing at the level of the pelvoureteral junction avoids the existing infection, eliminating the upward spread thereof to cause pyelonephritis and the risk of bacteremia and urosepsis. The implantation of a direct line from a body surface port to enter the ureter at a high level through a side-entry jacket provides diagnostic and therapeutic access into the ureter without the risk of spreading an existing infection upward. In so doing, it gains the advantage of allowing ureteroscopic visualization and the eradication of uropathogens with an antiseptic drip, for example, weeks before an antibiotic is could be relied upon to have eradicated the infection.

FIG. 2 duplicates FIG. 30 in copending application Ser. No. 16/873,914, entitled Vascular Valves and Servovalves—and Prosthetic Disorder Response Systems in showing a prosthetic alternative route for the voiding of urine to which a wearer with intractable urinary incontinence, frequency, or nocturia, for example, can switch whenever, as during a public performance or during a business gathering, or when otherwise unable to access a bathroom, or to which a wearer with nocturia can redirect voiding into a collection bag before going to bed.

In such a situation where ductus side-entry valve 143 is already in position at a level proximate to the pelvoureteral junction, a secondary, or sideline, such as an service or accessory channel to directly deliver medication or a maintenance substance through the valve and into the line are easily added, and would likely be a standard feature through which an implanted microcontroller chip would periodically direct the release of a stone solvent. Should the lower tract already be affected by disease or become diseased, the addition of the service or accessory channel would allow the lower tract to be bypassed and thus left without the passage of urine to wash away medication and interfere with healing.

Moreover, relatively caustic, urine is an irritant to tissue, the more so when inflamed or having undergone a surgical procedure. Bypass also facilitates viewing of the urothelial lining with the aid of an endoscope or intravascular ultrasound probe or treatment of the lower tract with an excimer laser, for example, while it remains dry both during and following the procedure. Combining the diversion mechanism of FIG. 2 with a prosthetic disorder response system would involve placing sensors to signal the controller of the need for the controlled release of medication were the urothelium to show signs of inflammation, infection, metastatic conversion leading toward malignancy, or established malignancy, for example.

A nonswitchable version of much the same system, also described and illustrated in copending application Ser. No. 16/873,914, serves as a prosthesis for patients missing parts of the lower urinary tract which not only incorporates features that make it more amenable to constant monitoring and the automatic inception of remedial measures by a fully implanted prosthetic disorder response system than might an ileal conduit and stoma, but eliminates the susceptibility to degenerative disease and much of the unhygienic aspects of a stoma responsible for irritation and infection associated with an exclusively organic rather than synthetic system. Inorganic materials are not susceptible to infection or to metaplastic degenerative transition to malignancy, require no blood supply or drainage, and accordingly unsusceptible to any inadequacy thereof.

The ability of a prosthetic disorder response system to administer its own maintenance serves to dispel the breakdown of tissue that would otherwise eventually lead to leaks at synthetic-tissue interfaces. While there are no synthetic materials that can assure the avoidance of an adverse tissue reaction entirely, the institution of a slow inflammation and infection counteractant drip, typically a steroid with an antimicrobial, periodically replenished at the reservoir such as 47 in FIG. 1, released through the connector such as 61 through an service or accessory channel, will alleviate this problem, so that the tissue adhesion or anastomosis provided by a tissue-tissue junction created in conventional repair loses much if not all of its special advantage.

Significantly, this means that the use of synthetic materials supported by direct from the body surface port drug targeting eliminates the need for the preliminary harvesting of gut for use as an ileal conduit, for example, in a preliminary operation itself susceptible to multiple adverse sequelae both at the harvesting site and in the reconstruction. Intestine is intolerant of the caustic effect of urine passing over the lining or mucosa of the lumen, and a prosthesis made of synthetic materials eliminates the need to assure adequacy of blood supply and drainage for the graft, as well as the possibility for it to become infected or undergo degenerative metaplasia that left untreated would eventually result in carcinoma.

When no preceding placement of an implant exists so that the introduction of a line to the ureter or bladder, for example, would raise hesitancy as initially invasive, the idea of implanting a line from a subcutaneous (subdermal) port in the pectoral region, then routed subcutaneously to a ureter is less likely to meet with resistance if it is stressed that:

1. Pathogens sometimes retreating to site of respite in the face of an antimicrobial only to recrudesce, placement thus is not for isolated but rather frequent instances of infection resistant to treatment,

2. The implantation procedure is safe and without significant complexity,

3. Ureteroscopy that must pass through infected urothelium spreading the infection toward the kidneys, entry into a ureter at a level superior to an active infection, to include the initiation of an antiseptic drip, allows ureteroscopic diagnosis and therapy before the infection has been eradicated with antibiotics which takes weeks where the infection is otherwise eradicated immediately without the risk of urosepsis, and

4. Left available for alternative use, it is almost always preferred not to remove such a line once introduced. Nevertheless, if preferred, the line is easily removed.

FIG. 3 duplicates FIG. 16 in copending application Ser. No. 15/998,002 entitled Ductus Side-entry Jackets and Prosthetic Disorder Response Systems, where connection to the left anterior descending coronary is meant to be understood as analogous to connection of the ductus side-entry jacket to a level along the ureter proximate to the pelvoureteral junction. In FIG. 3, part number 11 is a mainline shown in relation to the left anterior descending but no less suitable for passage of a ureteroscope into a ureter from well above the infected area and 13 is an service or accessory channel, or sideline, suitable for instilling medication such as antimicrobial or anti-inflammatory into the ureter.

This ‘two front attack,’ topically from a position higher up on the ureter and optionally through the blood supply by means of an oral antibiotic eradicates a lower urinary tract infection more promptly than might the latter alone. Accordingly, the arrangement depicted in FIG. 1 without an infection counteracting component can be supplemented with the addition of a direct drug pipeline led to the pelvoureteral junction as shown in FIG. 3. In most instances, the need for additional lines running from a subcutaneous (subdermal) portacath, or mediport, in the pectoral region and led to a level high up on the ureter would be apparent at the outset and included upon initial placement. The placement of a ductus side-entry jacket to pass a scope and service or accessory channel to drip medication into a ureter is not represented as justified for response to a routine isolated infection but rather only when infection of the lower tract has been repetitive so that decisive remedial action would best not be deferred, and the infection is refractory to conventional treatment.

Urosepsis life-threatening, it warrants emphasis that a permanent drip of a universal antimicrobial and biofilm dissolution agent such as highly dilute sodium hypochlorite (common bleach) with a topical anesthetic to dispel a stinging sensation, instilled from above the level of infection through connection to the ureter in the same manner as shown in FIG. 3 where connection by analogy is to the left anterior descending coronary should prevent all future infection whether caused by uropathogenic bacteria, viruses, or fungi, and without the patient compelled to drink water throughout the day, to which many, probably most, patients are averse and do not comply.

Conventionally, access to the upper tract is retrograde, hence, through the excretory, contaminated urethra. The use of an antiseptic with the antegrade approach provided by body surface port through sterile mainline 11—made possible by a ductus side-entry jacket to the upper ureter rather than through the urethra is critically superior in eschewing pathogens. Whether in combination with or apart from the urinary collection system shown in FIG. 2, the positioning of a ductus side-entry jacket as shown in FIG. 3 allows not only the antegrade insertion superior to the level of infection of a ureteroscope, but allows the delivery into the upper ureter of an antiseptic at the same time that it effectively eliminates the contingency of sweeping pathogens up toward the kidney by ureteroscopic spreading as a major problem with retrograde insertion. However sterile the ureteroscope was prior to use, acquired during retrograde insertion, contamination is difficult if not impossible to avoid.

The use of an antiseptic lubricant does much to dispel the risk of spreading the contamination upward, but is not completely dependable. In fundamental contrast to this deterrent, passage of the ureteroscope through the sterile line 11 in FIG. 3, wetted with an antiseptic eliminates contact with the urothelium. Neither antiseptic and lubricant coated ureteroscopes nor protective sheaths dependably prevent contamination (“Complications following retrograde intrarenal surgery (RIRS) vary considerably, and small-diameter ureteral access sheaths are reportedly significantly associated with rates of infectious complications following RIRS.” (Zhang, H., Jiang, T., Gao, R., Chen, Q., Chen, W., Liu, C., and Mao, H. 2020. “Risk Factors of Infectious Complications after Retrograde Intrarenal Surgery: A Retrospective Clinical Analysis,” Journal of International Medical Research 48(9):0300060520956833; see also, for example, Fan, S., Gong, B., Hao, Z., Zhang, L., Zhou, J., Zhang, Y., and Liang, C. 2015. “Risk Factors of Infectious Complications following Flexible Ureteroscope with a Holmium Laser: A Retrospective Study,” International Journal of Clinical and Experimental Medicine 8(7): 11252-11259; Mariappan, P. and Loong, C. W. 2004. “Midstream Urine Culture and Sensitivity Test is a Poor Predictor of Infected Urine Proximal to the Obstructing Ureteral Stone or Infected Stones: A Prospective Clinical Study,” Journal of Urology 171 (6 part 1):2142-2145).

The risk of urosepsis following retrograde passage of a ureteroscope through the lower tract to spread an existing infection further up toward the kidney is a major concern, one that serves as a deterrent to ureteroscopy, so that for conditions that recommend frequent visual examination and/or treatment at intervals, its avoidance a significant advancement (see, for example, Medina-Polo, J., Naber, K. G., and Bjerklund Johansen, T. E. B. 2021 “Healthcare-associated Urinary Tract Infections in Urology,” German Medical Science. Infectious Diseases 9:Doc05; Scotland, K. B. and Lange, D. 2018. “Prevention and Management of Urosepsis Triggered by Ureteroscopy,” Research and Reports in Urology 10:43-49; Cindolo, L., Castellan, P., Scoffone, C. M., Cracco, C. M., Celia, A., and 5 others 2016. “Mortality and Flexible Ureteroscopy: Analysis of Six Cases,” World Journal of Urology 34(3):305-310; Wagenlehner, F, M, Pilatz. A., Weidner, W., and Naber, K. G. 2015. “Urosepsis: Overview of the Diagnostic and Treatment Challenges,” Microbiial Spectrum 3(5); Sohn, D. W., Kim, S. W., Hong C. G., Yoon, B. I., Ha, U.S., and Cho, Y. H. 2013. “Risk Factors of Infectious Complication after Ureteroscopic Procedures of the Upper Urinary Tract,” Journal of Infection and Chemotherapy 19(6):1102-1108; Dielubanza, E. J. and Schaeffer, A. J. 2011. “Urinary Tract Infections in Women,” Medical Clinics of North America 95(1):27-41).

That “A Single Dose of Intraoperative Antibiotics is Sufficient to Prevent Urinary Tract Infection during Ureteroscopy,” Journal of Endourology 30(1):63-68 (Chew, B. H., Flannigan, R, Kurtz, M., Gershman, B, Arsovska, O., and 3 others 2016) is probably dependable in instances when infection is limited to pathogens previously cultured but not if presumed to have remained unchanged or unimpaired for a later procedure. The complement of pathogens may differ from one procedure to the next, and to obtain a culture prior to each session constitutes an impediment (see, for example, Karam, G, Chastre, J, Wilcox, M. H., and Vincent, J. L. 2016. “Antibiotic Strategies in the Era of Multidrug Resistance,” Critical Care 20(1):136).

In contrast, the release of an antiseptic through the device of FIG. 3 is automatic, intermittent, proceeds with the patient ambulatory and oblivious, and apart from an invasive session for each application throughout the period of treatment. (see, for example, Askim, Å., Moser, F., Gustad, L. T., Stene, H., Gundersen, T. M, and 5 others 2017. “Poor Performance of Quick-SOFA [quick sepsis related organ failure assessment] (qSOFA) Score in Predicting Severe Sepsis and Mortality—A Prospective Study of Patients Admitted with Infection to the Emergency Department,” Scandinavian Journal of Trauma, Resuscitative, and Emergency Medicine 25(1):56).

Most often contamination of the ureteroscope occurs during insertion (Scotland, K. B. and Lange, D. 2018, Op cit.), and as indicated, the path for insertion from above rather than up through the anatomy eliminates this contingency. In FIG. 3, service or accessory channel 11 allows the continued wetting of the scope with an antiseptic, and this continuity allows dispensing with concern that the ureteroscope is or was contaminated (see, for example, Bhojani, N., Miller, L. E., Bhattacharyya, S., Cutone, B., and Chew, B. H. 2021. “Risk Factors for Urosepsis after Ureteroscopy for Stone Disease: A Systematic Review with Meta-analysis,” Journal of Endourology 35(7):991-1000; Ma, Y. C., Jian, Z.-Y., Yuan, C., Li, H., and Wang, K. J. 2020. “Risk Factors of Infectious Complications after Ureteroscopy: A Systematic Review and Meta-analysis Based on Adjusted Effect Estimate,” Surgical Infections (Larchmont, NewYork) 21(10):811-822; Sun. J., Xu. J., and OuYang, J. 2020. “Risk Factors of Infectious Complications following Ureteroscopy: A Systematic Review and Meta-analysis,” Urologia Internationalis (Basel, Switzerland) 104(1-2):113-124; Morokuma, F., Sadashima, E., Chikamatsu, S., Nakamura, T., Hayakawa, Y., and Tokuda, N. 2020. “The Risk Factors of Febrile Urinary Tract Infection after Ureterorenoscopic Lithotripsy,” Kobe Journal of Medical Sciences 66(2):E75-E81; Huang, J., Zhao, Z., AlSmadi, J. K., Liang, X., Zhong, F., Zeng, T., and Wu, W. 2018. “Use of the Ureteral Access Sheath during Ureteroscopy: A Systematic Review and Meta-analysis,” PLoS [Public Library of Science] One 13(2):e0193600; Ofstead, C. L., Heymann, O. L., Quick, M. R., Johnson, E. A., Eiland, J. E., and Wetzler, H. P. 2017. “The Effectiveness of Sterilization for Flexible Ureteroscopes: A Real-world Study,” American Journal of Infection Control (St. Louis, Mo.) 45 (8): 888-895; Association of Perioperative Registered Nurses 2016. Guideline for Processing Flexible Endoscopes Sterilization and Disinfection, Denver, Colo.: Association of Perioperative Registered Nurses, pages 675-758; Lange, D., Bidnur, S., Hoag, N., and Chew, B. H. 2015. “Ureteral Stent-associated Complications—Where We are and Where We Are Going,” Nature Reviews. Urology 12(1):17-25; Chang, C. L., Su, L. H., Lu, C. M., Tai, F. T., Huang, Y. C., and Chang, K. K. 2013. “Outbreak of Ertapenem-resistant Enterobacter cloacae Urinary Tract Infections Due to a Contaminated Ureteroscope,” Journal of Hospital Infection 85(2):118-124).

Disposable ureteroscopes do away with preprocedural but not procedural contamination (Usawachintachit, M., Isaacson, D. S., Taguchi, K., Tzou, D. T., Hsi, R. S., and 3 others 2017. “A Prospective Case-control Study Comparing LithoVue, a Single-use, Flexible Disposable Ureteroscope, with Flexible, Reusable Fiber-optic Ureteroscopes,” Journal of Endourology 31(5):468-475). Accordingly, mainline 13 in FIG. 3 not only provides an alternative route for insertion of the ureteroscope, but antiseptic dripped from service or accessory channel, or sideline, 11 will eradicate pathogens when the ureteroscope is inserted at a level high up on the ureter. Contamination is routinely prevented by injecting dilute sodium hypochlorite into the body surface port. Pathogen sensors are addressed in copending application Ser. No. 16/873,914, entitled Vascular Valves and Servovalves—and Prosthetic Disorder Response Systems.

The same minimal set of components not part of a more encompassing microprocessor-administered program to treat comorbid disease with a ductus side-entry jacket as the end connector on a vessel allows intermittent infusion of a drug directly into the circulation to achieve much quicker dispersion than were the drug taken orally, by injection, or suppository. Copending continuation-in-part application Ser. No. 15/998,002, shows in FIG. 16, duplicated here as FIG. 3, just such an application, medication delivered directly into the left anterior descending artery. Other monomorbid applications not requiring hierarchical control are those discutient shown and described in copending application Ser. No. 15/998,002 entitled Ductus Side-entry Jackets and Prosthetic Disorder Response Systems, FIGS. 8, 13A, 13B, and 14 thru 16.

In a hierarchical control system, these subsidiary loops are nested, each level higher in the hierarchy integrating more comprehensive information, meaning information appurtenant of an additional symptom associated with the same morbidity or with an another morbidity tracked on a different channel, or arm, of the system. This information is then combined with the information on the channel of reference to integrate the two, and thus allow a determination as to the best resolution for the two taken together. At the highest level, the master control microprocessor commands remedial action that would best serve the reinstatement of normal homeostasis across the sum of morbidities.

Since remedial actions are isolated from one another by pipeline and electrical command targeting, interaction among the morbidities will seldom if ever be simple and direct but rather secondary consequences of the interdependencies among organs, tissues, and bodily systems attributable to neuroendocrine and autonomic interactions able to bypass direct physically isolated targeting. The type of end-effectors used follows from the disorder or disorders to be treated, and can include those electrostimulatory or otherwise neuromodulatory along with a rechargeable power source.

The number of potential configurations for such a system equals the number of serious chronic conditions and the combinations and permutations thereof, so that to describe a comprehensive set of specific systems in specific terms would take years. In such a fully automatic and fully implanted system, it is essential that all end connectors—ductus side-entry jackets, nonjacketing side entry connectors, vascular valves, and inline coupling jackets—fastened to vessels or to tissue surfaces can be depended upon not to leak, dislodge, fracture, break down, foul, clog, or otherwise fail, if not for the life of a pediatric patient, then for many years.

Such a system can be implanted to support any conventional surgical procedure that calls for the dispensing of supportive medication and follow-up monitoring as well as to initiate remedial action as necessary. Unless presenting complications, ordinarily straightforward and routine stitching procedures such as herniorrhaphies and excisional procedures such as varicocelectomies are not considered to warrant the placement of such a system.

Other common tissue end connectors include the ductus side-entry jacket placed in surrounding relation to a substrate native ductus such as a blood vessel, of which two appear here at the top of FIG. 4, having previously appeared as FIG. 32 in copending application Ser. No. 15/998,002, entitled Ductus Side-entry Jackets and Prosthetic Disorder Response Systems, of which such ductus connecting jackets were the subject. The choice of this figure is purely exemplary, any other organ or tissue having been selectable. A prosthetic disorder response system can be used to diagnose and treat any organ or tissue.

Vascular valves and servovalves as shown in copending application Ser. No. 16/873,914, entitled Vascular Valves and Servovalves—and Prosthetic Disorder Response Systems, are modified, hence, similar to, side-entry jackets but differ in incorporating means for dividing the flow-through cross section between either of two outlets. There are two types—those driven by a solenoid, which suddenly and fully extend a diversion chute into the lumen to divert all flow into a takeoff passageway, and those driven by a servomotor, which allow the gradual extension and retraction of the diversion chute to apportion flow between either of the two outlets.

Another type of vascular valve is the inline coupling jacket. This is a one-time destructive, or segment replaced excising, periductal collar placed at either end of a prosthetic replacement segment to replace the native segment when too diseased or malformed to be repaired. One example is a neonate born with a connective tissue disorder that has resulted in a very large aneurysm, interrupted aortic arch, or coarctation along the thoracic aorta for which no repair would prove durable and capable of growth. Another is the more familiar abdominal aortic aneurysm usually presented in an adult in whom an endoprosthesis poses the risk of an endoleak.

Under the restorative force of its spring hinges, the inline coupling jacket cuts through the ends of the native segment and rotates the prosthesis into its place in one continuous action so quick as to not interrupt the flow of blood through the substrate vessel. Both vascular valves and inline coupling jackets require clearance to place, and this can usually be attained with the aid of retractors. Where the distal end of a ductus plunges into anatomy too tight to access, such as the great vessels upon departing the heart, the distal jacket is placed as far distally as possible and the ductus distal thereto if susceptible to structural failure, is exceptionally protected with an endoprosthesis.

application Ser. No. 15/932,172, listed first below, led to the realization that rather than to draw drugs passing through the lumen to the level of the periductal collars described using magnetic means, the collars could be connected by catheteric piping led from implanted drug reservoirs implanted subcutaneously, or subdermally, at the body surface, usually in the pectoral region, to the substrate trouble spots, or nidi, where the reservoir outlet pumps are under the control of an implanted microcontroller to release drugs at the targets.

Targeted electrical or wireless lines can similarly be directed independently or in coordination with the release of drugs by the microcontroller or in more complex disease, a master control microprocessor. This avoids side effects not involving the target and the need for the considerably larger dosing required for systemic circulation with its exposure of nontargeted tissue, and eliminates the need for repeated invasive procedures to accomplish treatment for which the tools have been prepositioned and are automatically or remotely controlled.

The complete dependency of such a system upon ductus and tissue connectors that will remain intact, not migrate, leak, break or malfunction, and incorporate means for eliminating biofilm, clot, crystal, pathogens, and injury to the substrate ductus, as well as the desirability of showing uses for such connectors in cooperative arrangements under automatic diagnostic and therapeutic control prompted the next three applications—this because existing connectors not only omitted such capabilities but would actually work counterproductively to induce the degeneration of the substrate ductus or tissue so that the more these fine structures were exposed, the better.

Rather existing connectors were ‘dumb,’ not only in omitting these structural requirements but in failing to provide immediate accessibility for the control of diagnostic and therapeutic measures dependent upon fluid and electrical access. In contrast, the connectors shown in the last three applications allow the direct body surface port-to connector transcatheteric passage of miniature cabled devices such as an angioscope, laser, or linear or rotary thrombectomizer through the connector and into the substrate ductus, and the direct pipeline targeting of medication.

Diagnostic sensors, some specified below and many classified by analyte in copending application Ser. No. 16/873,914, entitled Vascular Valves and Servovalves—and Prosthetic Disorder Response Systems, can be incorporated into the connectors. When necessary, different nonmedicinal therapeutic mechanisms, such as electrostimulatory, laser, thermal, and radiation-emitting, can be mounted to if not incorporated into connectors controlled through wire- or wireless radio-transmitted commands.

Moreover, as delineated in copending application, Ser. No. 16/873,914, entitled Vascular Valves and Servovalves—and Prosthetic Disorder Response Systems, by incorporating a controllable diversion chute, ductus side-entry jackets can be made adjustable in apportioning flow between either of two outlet passageways, making possible the performing of several new surgical procedures, to include the administration of medication, semiautomatically and therefore accessible to a larger and more widely distributed number of surgeons. The applications implementing these concepts in chronological order were:

1. Integrated System for the Infixion and Retrieval of Implants, Ser. No. 15/932,172, largely concerned with the treatment of vascular and hematogenously disseminated disease;

2. Ductus Side-entry Jackets and Prosthetic Disorder Response Systems, Provisional application Ser. No. 61/959,560 filed on 27 Aug. 2013 and Nonprovisional application Ser. No. 15/998,002, concerned mostly with the design of blood and urine outlets into and inlets from catheteric fluid pipelines serving as shunts or bypasses;

3. Nonjacketing Side-entry Connectors and Prosthetic Disorder Response Systems, Ser. No. 14/998,495, now U.S. Pat. No. 11,013,858, occupied mostly with connectors fastened to the surface of tissue rather than in surrounding relation to a ductus; and

4. Vascular Valves and Servovalves—and Prosthetic Disorder Response Systems, Ser. No. 16/873,914, concerned with the design of valves that allow control over the flow of blood or urine.

Substantiation that in compressing and completely enclosing the fine vessels and nervelets entering and departing the adventitia of the substrate ductus or tissue actually promotes degenerative disease is prominent in the medical literature (see, for example, Arun, M. Z., Üstünes, L., Sevin, G., and Özer, E. 2015. “Effects of Vitamin C Treatment on Collar-induced Intimal Thickening,” Drug Design, Development, and Therapy 9:6461-6473; Kivelä, A., Hartikainen, J., and Ylä-Herttuala, S. 2012. “Dotted Collar Placed Around Carotid Artery Induces Asymmetric Neointimal Lesion Formation in Rabbits without Intravascular Manipulations,” BMC [BioMed Central] Cardiovascular Disorders 12:91; Nobécourt, E., Tabet, F., Lambert, G., Puranik, R., Bao, S., Yan, L., Davies, M. J., Brown, B. E., Jenkins, A. J., Dusting, G. J., Bonnet, D. J., Curtiss, L. K., Barter, P. J., and Rye, K. A. 2010. “Nonenzymatic Glycation Impairs the Antiinflammatory Properties of Apolipoprotein A-I,” Arteriosclerosis, Thrombosis, and Vascular Biology 30(4):766-772; Reel, B., Oktay, G., Ozkal, S., Islekel, H., Ozer, E. Ozsarlak-Sozer, G., Cavdar, Z., Akhisaroglu, S. T., and Kerry, Z. 2009. “MMP-2 and MMP-9 [matrix metalloproteinases]-alteration in Response to Collaring in Rabbits: The Effects of Endothelin Receptor Antagonism,” Journal of Cardiovascular Pharmacology and Therapeutics 2009 14(4):292-301; Kerry, Z., Yasa, M., Sevin, G., Reel, B., Yetik Anacak, G., and Ozer, A. 2005. “Diverse Effects of Calcium Channel Blockers in the Collar Model,” Acta Cardiologica 60(5):493-499; Nicholls, S. J., Dusting, G. J., Cutri, B., Bao, S., Drummond, G. R., Rye, K. A., and Barter, P. J. 2005. “Reconstituted High-density Lipoproteins Inhibit the Acute Pro-oxidant and Proinflammatory Vascular Changes Induced by a Periarterial Collar in Normocholesterolemic Rabbits,” Circulation 111(12):1543-1550; Donetti, E., Baetta, R., Comparato, C., Altana, C., Sartore, S., Paoletti, R., Castano, P., Gabbiani, G., and Corsini, A. 2002. “Polymorphonuclear Leukocyte-myocyte Interaction: An Early Event in Collar-induced Rabbit Carotid Intimal Thickening,” Experimental Cell Research 274(2):197-206; Bruijns, R. H. and Bult, H. 2001. “Effects of Local Cytochalasin D Delivery on Smooth Muscle Cell Migration and on Collar-induced Intimal Hyperplasia in the Rabbit Carotid Artery,” British Journal of Pharmacology 134(3):473-483; Crauwels, H. M., Herman, A. G., and Bult, H. 2000. “Local Application of Advanced Glycation End Products and Intimal Hyperplasia in the Rabbit Collared Carotid Artery,” Cardiovascular Research 47(1):173-182; Sozmen, E. Y., Kerry, Z., Uysal, F., Yetik, G., Yasa, M., Ustünes, L., and Onat, T. 2000. “Antioxidant Enzyme Activities and Total Nitrite/Nitrate Levels in the Collar Model. Effect of Nicardipine,” Clinical Chemistry and Laboratory Medicine 38(1):21-25; Herman, A., Matthys, K., Van Hove, C., Kockx, M., and Bult, H. 1999. “Oxidized Low-density Lipoprotein Enhances Intimal Thickening and Alters Vascular Reactivity,” Verhandelingen (Koninklijke Vlaamse Academie voor Geneeskunde van België) [Proceedings of the Belgian Royal Academies of Medicine] 61(1):19-38; Kerry, Z., Yasa, M., Akpinar, R., Sevin, G., Yetik, G., and 5 others 1999. “Effects of Nicardipine on Collar-induced Intimal Thickening and Vascular Reactivity in the Rabbit,” Journal of Pharmacy and Pharmacology 51(4):441-447; Yasa, M., Kerry, Z., Yetik, G., Sevin, G., Reel, B., and 5 others 1999. “Effects of Treatment with FK409 [((+/−)-(E)-4-ethyl-2-[(E)-hydroxyimino]-5-nitro-3-hexenamide], a Nitric Oxide Donor, on Collar-induced Intimal Thickening and Vascular Reactivity,” European Journal of Pharmacology 374(1):33-39; Van Put, D. J., Van Osselaer, N., De Meyer, G. R., Andries, L. J., Kockx, M. M., De Clerck, L. S., and Bult, H. 1998. “Role of Polymorphonuclear Leukocytes in Collar-induced Intimal Thickening in the Rabbit Carotid Artery,” Arteriosclerosis, Thrombosis, and Vascular Biology 18(6):915-921; Arthur, J. F., Yin, Z. L., Young, H. M., and Dusting, G. J. 1997. “Induction of Nitric Oxide Synthase in the Neointima Induced by a Periarterial Collar in Rabbits,” Arteriosclerosis, Thrombosis, Vascular Biology 17(4):737-740; Baetta, R., Donetti, E., Comparato, C., Calore, M., Rossi, A., Teruzzi, C., Paoletti, R., Fumagalli, R., and Soma, M. R. 1997. “Proapoptotic Effect of Atorvastatin on Stimulated Rabbit Smooth Muscle Cells,” Pharmacological Research 36(2):115-121; De Meyer, G. R., Van Put, D. J., Kockx, M. M., Van Schil, P., Bosmans, R., Bult, H., Buyssens, N., Vanmaele, R., and Herman, A. G. 1997. “Possible Mechanisms of Collar-induced Intimal Thickening,” Arteriosclerosis, Thrombosis, and Vascular Biology 17(10):1924-1930; Matthys, K. E., Van Hove, C. E., Kockx, M. M., Andries, L. J., Van Osselaer, N., Herman, A. G., and Bult, H. 1997. “Local Application of LDL [low density lipoprotein] Promotes Intimal Thickening in the Collared Carotid Artery of the Rabbit,” Arteriosclerosis, Thrombosis, and Vascular Biology 17(11):2423-2429; Ustünes, L., Yasa, M., Kerry, Z., Ozdemir, N., Berkan, T., Erhan, Y., and Ozer, A. 1996. “Effect of Verapamil on Intimal Thickening and Vascular Reactivity in the Collared Carotid Artery of the Rabbit,” British Journal of Pharmacology 118(7):1681-1688; Van Put, D. J., Van Hove, C. E., De Meyer, G. R., Wuyts, F., Herman, A. G., and Bult, H. 1995. “Dexamethasone Influences Intimal Thickening and Vascular Reactivity in the Rabbit Collared Carotid Artery,” European Journal of Pharmacology 294(2-3):753-761); Arthur, J. F., Dusting, G. J., and Woodman, O. L. 1994. “Impaired Vasodilator Function of Nitric Oxide Associated with Developing Neo-intima in Conscious Rabbits,” Journal of Vascular Research 31(4):187-194; Reckless, J., Fleetwood, G., Tilling, L., Huber, P. A., Marston, S. B., and Pritchard, K. 1994. “Changes in the Caldesmon Isoform Content and Intimal Thickening in the Rabbit Carotid Artery Induced by a Silicone Elastomer Collar,” Arteriosclerosis and Thrombosis 14(11):1837-1845.

Accordingly, these preliminary applications laid the groundwork for fully implanted automatic ambulatory prosthetic disorder response systems for which an unavoidable prerequisite are ductus and tissue connections that are secure, supported by service or accessory channels that directly pipeline-target maintenance solutions and drugs to the junctions and the tissue to which they are connected, and withal dependable for years if not for the life of a younger patient. Ductus connected thus can therefore thwart clogging due to a buildup of clot, crystal, or biofilm as well as eradicate the pathogen that deposited it. All devices and procedures described in these applications have been devised for use without the need to arrest blood flow or general anesthesia.

The availability of such systems will not only facilitate medicine and surgery as currently practiced but retroactively prompt and implement the adaptation of existing as well as recommend new therapeutic, diagnostic, and surgical techniques. In more advanced applications, a prosthetic disorder response system can considerably automate transplantation and prosthesis replacement procedures, notably, compound bypass solid organ transplantation—the seamless switching of the blood supply and drainage from the native organ of the recipient to and through that of the donor, thus transferring the graft organ from the circulatory system of the donor into that of the recipient. The ability to do this depends upon the availability of vascular valves, of course.

Development of Automated Diagnostic and Therapeutic Routines

In the treatment of complex comorbid disease, provided with a selection of alternative drugs chosen to determine which combination of these will prove most efficacious, the system optimizes treatment empirically and provides diagnostic information. When the patient is not confined to the clinic, this process can proceed without his awareness. In the same subject, the drug release program introduces a pause between successive differently paired drug releases to the same targets of sufficient duration to minimize overlap effects due to latency or the lingering of residues, that is, to significantly reduce if not eliminate the contingency of misattributing results actually caused by the carrying over of drugs used in a previous release.

Where more immediate results allow the extrapolation of longer term effects with confidence, this period may be reduced. Successive pipeline-targeted pairing of drugs released to different or to the same targets may therefore be much shortened. Continuously monitoring drug release and sensor feedback, the system may be used for diagnostic as well as for therapeutic uses. Broadly, either use is always implied, therapeutic results indicating pathophysiological processes, and the reverse. An object to keep to the minimum the number and dosing of drugs used, where the most efficacious agents might change as the morbidity or morbidities subside, the system performs staged re-checks as might allow the dosing, number of drugs, and their identity to be changed.

The ability of such a system to fine tune a drug regimen from the standpoint of the specific comorbid patient, or theranostically, is considerable. Direct pipeline targeting allows the results provided by the combinations of drugs used at the targets to be evaluated in substantial isolation from the side effects that might otherwise arise were systemic administration to give any significant exposure to intervening tissue and compresence in the circulation that could result in the interjection of unintended pharmacokinetic, pharmacodynamic, or metabolic effects.

Side effects can affect the responsiveness of tissue to another drug, pharmacokinetic effects can affect the uptake, metabolism, and excretion of another drug, and side effects due to either or any of multiple drugs in combination can alter what targeted would instead be nonconflated results in the short term, although over time, affecting one major organ may effectuate alteration in the function of others. Such later term effects, such as evidenced in the cardiorenal syndrome, are secondary rather than side effects.

Qualification thus invalidates much of the reliability and therewith, the applicability of automatic intelligence therapeutic decisions based upon the conventional, or nontargeted, administration of drugs, substantially overturning much but certainly not all, of what is accepted as established knowledge concerning even familiar drugs. In this fundamentally altered circumstance, the implanted system must itself be used to generate the diagnostic and therapeutic results suitable for its uses. This is not to say that the determination as to which drugs to consider for a familiar condition is completely unknown.

At least with the present state of medical diagnostics, the development of a prescription-program for a specific patient cannot commence on the basis of signs and symptoms provided by sensors throughout the body. Rather it must commence against a well developed background of decision making on the part of experienced clinicians that allows pertinent sensors to be placed on the basis of experience at the outset to the exclusion of numberless irrelevant ones as if these were limited to pure guesswork (see, for example, Mark, D. B. 2005. “Decision-making in Clinical Medicine,” in Harrison's Principles of Internal Medicine, New York, N.Y.: McGraw-Hill, 16th Edition, pages 6-13).

Drug delivery automated or not, that the substantial elimination of side effects accomplished by eliminating nontargeted tissue through segregated targeting exerts a significant simplifying effect on therapeutic decision making is largely due to the avoidance of uncertainty created where an ability to segregate drug is lacking. That is, the capabilities of the system to evaluate both the efficacy of each drug with respect to its target and of the drugs combined for the overall health of the patient when mixed together throughout the circulatory system lends clarity to and even an ability to optimize these interactions.

Viewed thus, a hierarchical analysis that pinpoints the problematic interactions among numerous drugs circulating in the blood simultaneously and the effect of substituting the drugs implicated yields significant information for pharmaceutical application in any context. Once identified, only drugs in such a mix that need not be allowed unlimited access are directly pipeline targeted. Drugs to localized and circumscribed sites of disease are pipeline targeted, but disease with the ability to spread to other parts of the body, notably, metastatic cancer, demand circulation-wide dispersal to destroy ‘daughter cells’ released from the primary or ‘mother’ tumor.

The original site is then pipeline-targeted but is accompanied by a systemic or global background dose. Notoriously damaging to nontargeted tissue and unavoidable, chemotherapeutics demand a background systemic dose; however, depending upon the dose necessary to kill any shed, or ‘daughter’ cells, by segregating the dose targeted, the relative size of that targeted and that dispersed may be adjustable to allow the latter to be reduced to do less damage Deng, G., Sun, Z., Li, S., Peng, X., Li, W., and 4 others 2018. “Cell Membrane Immunotherapy Based on Natural Killer Cell Membrane Coated Nanoparticles for the Effective Inhibition of Primary and Abscopal Tumor Growth,” American Chemical Society Nano 12(12):12096-12108).

The essential function of any given drug may not be so clear as to reveal potential off-label applications, but is known to the extent of its essential function as metabolic, anti-inflammatory, antimicrobial, anesthetic, and so on and is known in relation to different diseases. However, since remote effects are not necessarily predictable, this calls for translational research with sensors applied to every bodily system essential to detect unanticipated consequences. To completely isolate the delivery of drugs best preserves the nominal, or abstract, effect of each. That is, the mixing of drugs together in the circulatory system is the primary cause for most of the uncertainties as to their interactions and the effect of these interactions upon different metabolic pathways of which the consequences might eventuate at unpredictable sites.

The same applies to combination drugs such as amoxicillin/clavulanate. It is the secondary, the effects further remote, such as that of a positive inotrope released directly into the coronary arteries as illustrated in copending application Ser. No. 15/998,002 entitled Ductus Side-entry Jackets and Prosthetic Disorder Response Systems, FIG. 16 on the kidneys, for example, that less predictable, warrants close study. Were such research initiated before the era of modern evidence-based medical practice and the development of understanding as to the interdependence among the organs, months if not years might be needed before the dependable results as to the interdependence among the organs appeared. By now, however, cardiorenal disease, for example, its causes, and course are substantially understood, and the elimination of the complications imposed by eliminating the meeting of drugs in intervening tissue, introducing extraneous consequences, simplifies the situation.

While undependable from the standpoint of dosage, or quantitatively, the findings of existing artificial intelligence programs should nevertheless define a set of drugs most likely to prove efficacious in the targeted treatment of disease when concurrently targeted in combination to the same site or separately to different sites. The development of therapeutic routines is thus facilitated by taking the results of automatic intelligence to the extent of identifying suitable drugs and testing these for the targeted delivery of what will likely prove to be the same drugs but in relatively tiny, more concentrated doses and formulated in the posthepatic and postrenal pass form compared to the prethepatic and prerenal pass systemic administration form, the results of this process inherently developed in the context of treating specific patients.

As with any disease or combination of medical problems, the governing object is to identify the fewest drugs in the smallest doses that proves most efficacious. Automatic diagnostic routine programs to include event recording and pauses between rechecks can be uploaded into the implanted master control microprocessor through a universal serial bus or ethernet socket, for example, incorporated into the body surface port at the same time that the drugs are loaded into the implanted drug reservoirs. Numerous universal serial bus type cable connector sockets incorporated into a body surface port allow the addition or removal of prerecorded or keyed in code to support a routine or subroutine.

The control system assures adherence to the prescription-program. Relatively minor adjustments thereto can be applied as indicated in the clinic, or if the patient is remote, then changes to the prescription-program are transmitted to the cellular telephone of the patient or to that of an adult and incorporated from the phone into the existing program through the socket in the body surface port. A large drug selection and sequencing mechanism can be simply a large embodiment of the body pack rotatory indexing turret or turntable type shown in FIG. 4, which can be housed in a refrigerated enclosure and/or stored in a refrigerator.

The exceptionally targeted rather than conventionally nontargeted circumstance for the administration of drugs notwithstanding, the selection of drugs still adheres to clinical experience—drug identification does not recommence from the outset—practitioners already know which drugs and combinations thereof are most effective in treating various diseases. Where that drug or drugs optimal cannot be predicted—which in more complex comorbid disease would be more of a factor without automation—or more likely, the altered circumstance makes dosing in various combinations unclear—the microprocessor systematically assesses, that is, continuously ‘tunes in’ the relative efficacy of the various combinations sequentially based on the sensor inputs for the combination at the pertinent hierarchical node as the dose of each drug and combinations thereof are gradually increased.

For later term or secondary ‘side effects’ such as cardiorenal or cardiohepatic syndrome that would not appear promptly upon or shortly following administration, if more than a few drug combinations must be tested, this process will require continuation on a weekly, or even monthly basis; however, the patient is provided with the best combination of drugs identified at each visit. Initializing the prescription-program is best done in the clinic, where the number of drugs and the need to implant a large number of reservoirs is avoided. There a refrigerated drug console can house any number of drugs for delivery in combination.

The application of hierarchical diagnostic and therapeutic control and the distinction between directly pipeline targeting of drugs to the sites of disease as opposed to the systemic administration of drugs should not be conceived of as indissociable. In fact, because the results of drug interactions are short term in emerging, hierarchical diagnostics is more quickly responsive and productive of data when drug delivery is not segregated. However, from the standpoint of the longer term consequences among the organs, the immediate data may be viewed as extraneous, the counter to which is that some drug-drug interactions and side effects may actually offer some benefit, if not in the specific condition tested, then possibly in another.

Absent the direct pipeline targeting of drugs—that is, administering drugs by any route that disperses these throughout the circulation must result in drug-drug and drug-food interactions. That except for the possibility of nugatory trace amounts, multiple drugs can be targeted to different objectives in complete isolation from one another imparts considerably expanded complication-reduced or eliminated applicability to each, making possible numerous uses and combined uses not previously permissible. In many instances, the diagnostic regime intrinsic in the hierarchical control system will yield interactive data in relatively little time compared to waiting for the appearance of long term consequences which would eventually appear in each organ and among organs. From this standpoint, the use of hierarchical diagnostics provides more information concerning drugs not pipelined.

A more expansive view is that applying the same hierarchical diagnostic system to a comorbid condition first without, then with pipelining, clarifies the effect of drug isolation up to the target blood supply and allows the relative advantages in pipelining with respect to different combinations of drugs targeted to different targets. If this information has been previously determined, it may elucidate which if any drug-drug interactions are of nugatory if any consequence and therefore which pipelines can be omitted and the drugs administered systemically whether by injection, infusion, or by mouth. Just as drugs should always be minimized in number and dose, growing data that establish certain pipelines as ineffectual will allow these to be eliminated.

The drug replenishment console is plugged into the body surface port to replenish the implanted reservoirs no differently than when with a multiple head hypodermic syringe or jet injector such as those shown in FIGS. 27A and 27B of copending application Ser. No. 16/873,914, entitled Vascular Valves and Servovalves—and Prosthetic Disorder Response Systems respectively is used. Prescribed load lists for stocking such units is developed on the basis of antecedent experience where more immediate or short term side effects and drug interactions are disregarded, as well as system responsive experience gained up to that date in treating the different comorbidities. The execution and its step-by-step results are recorded and available for output through the same one socket in the body surface port, for example. Problematic drugs are then preferably substituted with others, and if necessary, relegated to pipeline-targeted delivery.

Compound bypass, or switch, solid organ transplantation is described and illustrated in copending application Ser. No. 16/873,914, entitled Vascular Valves and Servovalves—and Prosthetic Disorder Response Systems. Semiautomatic vascular segment replacement pertains to larger blood vessels and their branches. Both solid organ recipient to donor switching and prosthetic ductus segment insertion require not only vascular valves and servovalves and inline coupling jackets respectively, but all other tissue and ductus connectors in the disorder response system must be designed no to leak, migrate, abrade neighboring tissue, or otherwise cause complications for years if not for the life of the patient.

Currently, the major drawbacks to organ transplantation when any other treatment would represent a halfway measure are time-limited sufficiency and the lifelong need for immunosuppressive medication. For this reason, transplantation is said to represent an exchanging of one disease for another—a nuisance in a competent and a menace in an incompetent patient. Another deterrent is the frequently limited life of the graft organ before it must be replaced.

To these objections, it is responded that metered compound bypass, or switch transplantation as delineated in copending application Ser. No. 16/873,914, entitled Vascular Valves and Servovalves—and Prosthetic Disorder Response Systems eliminates the anoxic and most of the surgical and immunological trauma associated with conventional transplantation, and that the continuous surveillance and immediate release of medication as necessary by the automatic response system with the patient oblivious to its silent operation eliminates dependency upon prescription adherence. Transplantation thus requires vascular servovalves as described in copending application Ser. No. 16/873,914.

Essential for an automatic system are blood vessel and drugline end connectors and vascular valves designed for stability and security that allows permanent placement. Tissue irritation and breakdown must be minimized, means incorporated to dispel adverse reactions, and blood and drug delivery feedlines must remain intact and leak-free. These prerequisites are addressed in copending application Ser. No. 15/998,002, entitled Ductus Side-entry Jackets and Prosthetic Disorder Response Systems and in U.S. Pat. No. 11,013,858, entitled Nonjacketing Side-entry Connectors, and Prosthetic Disorder Response Systems.

The simultaneous release into the circulation of more than one conventional drug or nanoparticulate-coated homing, or docking, agent such as addressed below would make possible interactions between or among these no less than does the risk of adverse drug-drug interactions between and among conventional drugs. Even if time allows avoiding concurrent administration, any non-uptaked residue or its breakdown products might linger in the blood stream.

Direct pipeline targeting of drugs is not a halfway but intentionally brute force measure to route the targeting of medicinal agents to different specific targets so that each is isolated from the others. Where comorbid disease and exigency necessitate that multiple targets be treated simultaneously, each agent, whether conventional or docking, is isolated from each of the others and terminated in the lesion or affected volume of tissue targeted.

The approach invites disparagement on four bases:

1. A lack of chemical sophistication in disregarding the intrinsic affinity for the target of the agent chosen which need not be self-seeking of the target.

2. A misconceived view of the implantation of drug targeting pipelines as crude and needlessly traumatizing.

3. Implants can provoke adverse tissue or foreign body reactions.

4. Mechanical defects in the connectors used could result in leaks, creating discrepancies in effective doses, and allowing contents exposure to nontargeted tissue and other drugs, effectively negating the object in such a closed pipeline mechanical approach in the first place.

However, this approach eliminates drug-drug interactions that serve to needlessly complicate and interfere with treatment, and system parts are placed endoscopically through one or more ‘keyhole’ incisions without the deterrent of general anesthesia. Moreover, all jackets and connectors incorporate service or accessory channels and can be supported by other jackets by dispensing anti-inflammatories or antimicrobials to dispel adverse tissue reactions and prevent infection.

That the automatic targeted release of immunosuppressives, antimicrobials, and other maintenance drugs, and the vascular valve-implemented anoxia-free compound bypass procedure make it possible to directly transfer the graft organ from the circulatory system of the donor into that of the recipient should materially extend the life of the transplant. Metered compound solid organ transplantation eradicates the deterrents to transplantation using conventional methods, to include the lack of a backup supportive system equipped with rejection and other analyte sensors which leaves the patient, especially early in development, in an irreversible suboptimal condition.

For these reasons, the familiar comment that an organ transplant only exchanges one disease for another is effectively dispelled. Heart transplantation using a compound bypass technique is fundamentally superior to any conventional method from every standpoint. By placing the donor on life support before dying and directly transferring the graft organ from the circulatory system of the donor into that of the recipient, the heart is never subjected to the shock of death, circulatory arrest, or ischemia-reperfusion injury.

When tissue engineers gain the ability to generate a fully functional replacement organ from stem cells harvested from the patient, the problem of how to insert the new organ in place of the old with minimal trauma and then how best to support the graft organ following placement will remain. Then, genetic matching having been omitted from the problem, the relatively low trauma of sudden switch transplantation implemented with solenoid driven valves, followed if and only if necessary, by the continuous monitoring and medicinal support of a fully implanted automatic prosthetic disorder response system, will assure the suppression of atherosclerotic degeneration and graft organ durability.

Until then, genetic matching will remain the major cause for late if not acute rejection, a problem that metered compound bypass, or switch, transplantation with followup by an implanted response system can ∧variable servovalves adjusted gradually by the implanted control microprocessor to sustain the relatively silent donor-recipient reciprocal cross circulatory microchimerization that without preprocedural cross transfusions, for example, is momentary in a sudden switch transplant. By extending the duration of reciprocal cross circulation well beyond what it would be in a sudden switch transplantation, metered switch transplantation assists to induce immune tolerance between the donor and the recipient gradually enough to minimize if not avert a rejection reaction more likely to ensue when exposure to alien tissue arises by abrupt confrontation.

This subdued or relaxed approach may be preceded, accompanied, or preceded by the conventional administration of immune tolerance inducing medication to include the gradual exchange of tissues between the donor and the recipient. A key benefit in the gradual exchange and blending of the blood of donor and recipient is that there is no stoppage in the flow of blood as the graft organ is entered into the circulatory system of the recipient.

Only once the graft organ has been completely transferred into the circulatory system of the recipient is the innate organ of the recipient removed and the graft organ harvested, that is, cut free from its stumps and positioned orthotopically in its place. When the graft organ is the heart, measures are provided to continue circulation in the donor to best preserve the remaining organs and tissues. When the innate organ is impaired but salvageable, the blending of blood between the donor and recipient is left at the half way point and the graft organ added to assist that native. Such an approach eliminates the need for assist devices and artificial hearts which are not able to sustain a younger patient to the end of life and come at a cost beyond that affordable throughout much the world.

In contrast, supported by the implanted prosthetic disorder response system, two reasonably functional hearts working together can discharge an ejection fraction, or absolute stroke volume, sufficient to sustain the patient indefinitely. Doubling thus is no less applicable to the other solid organs and other structures.

The ability to include imperfect replacement organs to work together with those native considerably expands the pool of available transplant organs. As organic assist devices, these avoid the high cost of mechanical assist devices and an implicit limited term in viability with the potential to bring relief throughout much of the world.

Copending application Ser. No. 16/873,914 entitled Vascular Valves and Servovalves—and Prosthetic Disorder Response Systems. provides detailed and fully illustrated descriptions of the sudden and metered compound bypass, or switch, methods for solid organ transplantation, with illustrations that depict in detail the transplantation of a heart. Also shown are various positions for the second heart with ancillary devices as necessary. The elimination of trauma to the graft organ—and if retained for additional graft organs, the organs of the deceased donor—is acutely beneficial in sparing the shock of excision and insertion into an immunologically alien milieu.

A conventional heart transplant involves excising the donor heart, often storing it in a chilled perfusate, then cutting it approximately in half to remove the ventricles, then stitching this replacement anterior donor portion to the front of the innate posterior portion of the recipient heart. Transplantation using the compound bypass technique necessitates the use of vascular servovalves as described in copending application Ser. No. 16/873,914.

Compared to a metered compound bypass heart transplant, which calls for no incision into the donor or recipient organs or interruption in the flow of blood through either, the conventional procedure involves mutilation contrary to these safeguards that increases the need for postoperative diligence and timeliness in maintenance, calls for higher doses of immunosuppressants, and which pursued to perfection will unavoidably contribute to early graft breakdown and rejection. With donor life support initiated prior to death and spontaneous circulation and respiration sustained, the gradual transfer of the graft organ from the circulatory system of the donor into that of the recipient with no interruption in full perfusion should considerably reduce if not eliminate an integrated stress response which the conventional method induces through hypoxia and glucose deprivation and not just trauma.

Moreover, while less traumatic and extended insults than a conventional heart transplant provoke such a response, these will usually subside so that the response does not progress from self-protective to self-destructive (“Although the ISR [integrated stress response] is primarily a pro-survival, homeostatic program, exposure to severe stress can drive signaling toward cell death.”—Pakos-Zebrucka, K., Koryga, I., Mnich, K., Ljujic, M., Samali, A., and Gorman, A. M 2016. “The Integrated Stress Response,” European Molecular Biology Orgnization Reports 17(10):1374-1395). Following the conventional harvesting of a solid organ, the self-destruction does not subside—it transitions into irreversibly greater self-destruction. The injury induced as the result of the integrated stress response and ‘cytokine storm’ affects not only the specific transplant organ or graft but all the organs and tissues of the deceased donor maintained on life support as the prospective source for other transplant grafts.

That is, the stresses of death and organ excision imposed by conventional solid organ transplantation are not just pertinent to the condition of the immediate graft organ as such but to all of the organs and tissues of the deceased donor which are degraded for premature rejection (see, for example, Diaz-Bulnes, Saiz, M. L., Lopez-Larrea, C., and Rodriguez, R. M. 2029. “Crosstalk between Hypoxia and ER [endoplasmic reticulum] Stress Response: A Key Regulator of Macrophage Polarization,: Frontiers in Immunology 10:2951; Pakos-Zebrucka, K., Koryga, I., Mnich, K., Ljujic, M., Samali, A., and Gorman, A. M 2016, Op cit.; Wang, M. and Kaufman, R. J. 2016. ‘Protein Misfolding in the Endoplasmic Reticulum as a Conduit to Human Disease,” Nature 529(7586):326-335; Shimazawa, M. and Hara, H. 2006. “Inhibitor of Double-stranded RNA-dependent Protein Kinase Protects Against Cell Damage Induced by ER [endoplasmic reticulum] Stress,” Neuroscience Letters 409(3):192-195; Kroemer, G., Guillermo; M., and Levine, B. 2010. “Autophagy and the Integrated Stress Response,” Molecular Cell 40(2):280-293; Molina, P. E. 2005. “Neurobiology of the Stress Response: Contribution of the Sympathetic Nervous System to the Neuroimmune Axis in Traumatic Injury,” Shock (Augusta, Ga.) 24(1)3-10; Harding, H. P, Zhang, Y., Zeng, H., Novoa, I., Lu, P. D., and 10 others 2003. “An Integrated Stress Response Regulates Amino Acid Metabolism and Resistance to Oxidative Stress,” Molecular Cell 11(3):619-633).

The initial stress response leads into a severe reaction, a systemic inflammatory response referred to as the polypeptide mediator release syndrome, hypercytokinemia, or infusion reaction, popularly referred to as a ‘cytokine storm,’ or cytokine storm syndrome, which likewise results in injury not only to the graft organ but to all other prospective donor organs and tissues of the deceased donor on life support (see, for example, Simkin, J., Strange, T., Leblanc, N., Rivera, J. C. 2021. “What Is a Cytokine Storm and Should It Matter to Me?,” (review) Journal of the American Academy of Orthopaedic Surgeons 29(7):297-299; Canna, S. C. and Behrens, E. M. 2012. “Making Sense of the Cytokine Storm: A Conceptual Framework for Understanding, Diagnosing, and Treating Hemophagocytic Syndromes,” Pediatric Clinics of North America 59(2):329-344; Gentile, L. F., Cuenca, A. G., Efron, P. A., Ang, D., Bihorac, A., and 3 others 2012. “Persistent Inflammation and Immunosuppression: A Common Syndrome and New Horizon for Surgical Intensive Care,” Journal of Trauma and Acute Care Surgery 72(6):1491-1501; Tisoncik, J. R., Korth, M J, Simmons, C. P., Farrar, J. Martin, T. R., and Katze, M. G. 2012. “Into the Eye of the Cytokine Storm,” Microbiology and Molecular Biology (review) 76(1):16-32) and systemic inflammatory if not hyperinflammatory response syndrome associated with organ failure and death will have been considerably suppressed and probably prevented from degrading the prospective graft.

Broadly, eliminating this deterioration reduces the multiple obstacles of transplantation to one of immune tolerance, and the metered switch, or compound bypass, method, described in copending application Ser. No. 16/873,914, entitled Vascular Valves and Servovalves—and Prosthetic Disorder Response Systems, reduces this remaining problem as well. Essentially, the graft organ is spared the trauma, and therewith, the ‘realization,’ that its host had died and that it had been transferred into an alien milieu.

In fact, surgical procedures controlled peri- and midoperatively to partially if not completely automate their execution are simplified as to remove these from the exclusive purview of a relatively small number of highly skilled and experienced specialists. Brought within the compass of general surgeons, access to such support, especially for rural and less developed world populations, is considerably increased. Given this virtually universal applicability that encompasses a profuse number of complex problems of internal medicine and surgery, examples cited must be limited, sufficient information provided to make it apparent how a system to treat a specific disease or combination thereof would be configured.

In monomorbid disease, the controller is a microcontroller; while in comorbid disease, it will usually be a microprocessor administering a hierarchical control program in which the microprocessor acts as the master controller executing a program in which each component morbidity is assigned to a channel, or arm, in a rising ladder of nodes, or levels of diagnostic data collection, with cross-node data integration accomplished at each level among the channels. Rising up a level then calls for integrating the information associated with the additional morbidity with that accumulated at a lower level for the morbidities as distinct.

That is, at subordinate levels in the hierarchy, the data in the different arms at the same level are cross-compared to identify the optimal treatment across the larger number of morbidities at that level. Each rising step extends this integration to include an additional channel. The master controller then integrates the information arriving up through the subordinate levels in the hierarchy and issues commands to achieve the most efficacious result for treating the combination of conditions. This does not, however, equate to the dispersal of drugs through the systemic circulation. Indeed, each release of a drug or other therapy is directly pipeline-targeted and can be substantially if not entirely isolated from any others, thus eliminating side effects due to a high degree of undesirable and indiscriminate cross exposure.

Despite the tightest targeting of drugs, the organs and tissues of the body represent a fully integrated system, so that the release of drugs must take into account not direct exposure but rather sequelary, or secondary, interaction among organs and tissues. Cardiorenal and cardiohepatic conditions, for example, represent just two of the common conditions of interaction attesting to the interdependence of all parts of the body mediated by the autonomic and endocrine as well as the circulatory system. Physiological interdependence thus is distinct from the similar affectation of distributed nervous tissue due to centralized defects in genes that govern neuroendocrine function as pertains to the paragangliomas in both the stomach and carotid bodies, for example, as well as defects in genes with pleiotropic substrates, or expression sites, remote from one another.

FIG. 1, previously published as FIG. 12A in U.S. Pat. No. 11,013,858, entitled Nonjacketing Side-entry Connectors and Prosthetic Disorder Response Systems, provides a schematic representation of a more complex system which includes a transdermal (or transcutaneous), battery charging secondary coil 64 and transdermal charging circuitry 50, as well as a battery 54, surface port 46 here positioned subcutaneously in the pectoral region, drug storage reservoir or reservoirs 47, miniature reversible pump 49, microprocessor 53, and drug delivery pipeline, or drugline 48, leading to side-entry connector 61.

For a patient with an intact bladder, conditions such as intractable frequency and/or incontinence can be eliminated with a nonjacketing side-entry connector positioned along the bottom of the bladder piped to an effluent outlet port positioned to the ipsilateral side of the mons publis to pass a tube down to a collection bag cinched about the ipsilateral thigh. For nighttime use when the patient will be horizontal to interfere with passive outflow due to gravity, a miniature infusion or peristaltic pump as used at the outlet of drug reservoirs is incorporated into the outlet pipe .51

Any pain caused by the anchoring needles pending their encapsulation with fibrous tissue is suppressed with a topical anesthetic such as lidocaine delivered through a connector service or accessory channel. Such a simple connector line, outlet port, and collection bag can be prepackaged with instructions for its endoscopic placement. None of the componently shown in the upper part of the drawing is needed for this simple application seen in FIG. 1.

Depending upon the condition or conditions to be treated, other components might include electrostimulatory or otherwise neuromodulatory, as well as warming, cooling, and/or pumping devices, for example. The positioning of such a system is for response to serious chronic, chronic intermittent, or episodic conditions or for surgical administration and/or surgical follow-up. The system can also continuously monitor and respond to any chronic disorder where the only surgical factor consists of emplacement of the system itself accomplished endoscopically through two or three ‘keyhole’ incisions of a few centimeters, drug delivery pipelines and electrical conductors tunneled subcutaneously and around, to avoid strangling, viscera with the aid, for example, of an ultrasound handpiece.

A totally implanted automatic prosthetic disorder response system can control the execution and then support a number of surgical procedures, some, such as a heart transplant, critical for survival in patients of all ages, and another the replacement of large vasculature so congenitally malformed that given the rate of growth in a neonate, no conventional means for its repair will prove satisfactory for more than a short time. Were the defect corrected once and for all, or at least for a period of years, the child would not be plagued and repeatedly debilitated with reoperations. The advancement this bodes for heart transplantation, executed using the compound bypass, or switch, method under the automatic control of the disorder response system warrants emphasis. Usually, the need for a new heart—or rather a part thereof—is due to ventricular failure of the native heart, necessitating replacement of the ventricles.

Unlike a kidney transplant, for example, where the graft organ is left intact and orthotopically positioned in place of the original or heterotopically, a heart transplant is really a hybrid repair that cuts off and takes the working part of the donor heart and removes the defective part of the recipient heart, then stitches the working parts of each together to make a working heart. A prosthetic disorder response system could be used to support this or any other conventional procedure; however, its emergence enables superior methods that allow the direct targeting of drugs to nidi without exposing unintended tissue, for example.

Where the conventional approach is to cut off the ventricles from the donor heart to replace the ventricles of the recipient heart, the compound bypass technique eliminates the need to cut into either heart. Thus, using a conventional technique, the donor heart is not used intact. Rather, both the native and donor hearts are more or less cut in half and then stitched together, so that the ‘transplant’ actually retains much of the recipient heart and consists of both. The need for immunosuppressives is no less applicable following a compound bypass transplant as set forth here. Incision into both the donor and recipient hearts not only traumatizes both severely, but disallows continuity of perfusion in either.

Circulation through the hearts necessarily withheld during this procedure, the reinstatement of perfusion causes further trauma to both hearts in the form of ischemia-reperfusion injury, strongly suspected to reappear as the cardiac allograft vasculopathy that almost inevitably results in graft failure. The limited life of the transplanted heart is to be expected: in conventional heart and other solid organ transplantation, the donor or graft organ is excised from its natural milieu after the host has died so that circulation has stopped with the organ then stored thus.

When the donor and recipient are not already at or readily transported to the same location, the cessation in circulation following remote harvesting and subsequent loss of perfusion may be ameliorated during transport of the graft organ with the aid of a normothermic ex vivo perfusion machine; however, the shock and trauma of death, excision, and interruption if not the loss of perfusion cannot be reversed. When as must often occur the donor is discovered after having died, or already hospitalized was not expected to expire and not prepared for post-mortem organ harvesting, it is better to deliver the body intact rather than the graft organ.

The new combination of severely traumatized and imperfectly matched hearts must then be protected at the expense of safety to the body as a whole through the administration of immunosuppressives. Conventional heart transplantation does not completely remove the native heart but rather replaces the ventricles with those taken from a healthy heart. This fixes the native and donor tissues in immediate interdependent contact, effectively left inseparable in the event of rejection or infection. That the immunosuppressives are dispersed throughout the circulation is yet another major insult.

A heart transplant with the support of such a system is a genuine transplant that orthotopically replaces or—in a compound bypass type double heart transplant—heterotopically supplements the native with a donor or accessory organ not sewn onto and therefore treatable separately from that native. Given the new option of removing an imperfectible heart from the recipient in its entirety and replacing not just its ventricles but the entire intact heart with a good intact one, an attempt to repair such a heart, along with use of mechanical assist devices, is properly relegated to a bridging action to sustain the patient until a good heart becomes available.

Unsurprisingly, a heart transplant accomplished using conventional methods usually requires retransplantation within one, less often, up to two decades, during which the patient must take immunosuppressives that produce an increased susceptibility to infection. If this regimen is not followed, the transplant will be rejected, and without the support of a mechanical assist device and retransplantation, the patient will die. Until then, infection, rejection, or both are fully capable of killing him. If surgical repair is a halfway measure, then as currently practiced, heart transplantation is also a halfway measure. Fundamental improvements in the transplantation procedure and its follow-up treatment, will, however, improve the results of a heart or any other solid organ transplant to become fully satisfactory.

With a prosthetic disorder response system, the administration of medication is automatic—in the case of a metered compound bypass heart transplant, having been administered by the same system that conducted the operation—so that cognitive impairment, negligence, or contumacy cannot result in a failure to adhere to the prescription. As with other medication best kept from unaffected parts of the body such as chemotherapeutic, the totally implanted prosthetic disorder response system tightly targets the bulk of such medication, sparing the rest of the body increased susceptibility to infection, making such a totally implanted automatic drug delivery system a major advancement in its own right.

The convenience and noninvasiveness of oral and every other conventional form of drug delivery is often gained at the cost of indiscriminate dispersal throughout the circulatory system that exposes nontargeted tissue which may lead to adverse side effects and requires dosage levels high enough to compensate for this degree of dilution. Absent an intrinsic affinity such as that of iodine for the thyroid gland, alternative routes are less convenient, but subject to the same shortcomings. In contrast, medical surgery consists of prepositioning prescription-responsive sensors and drug or other therapy-releasing components in support of a medical diagnosis. To emplace such a system is invasive, but falls far short of major surgery.

The aim in medical surgery is to position if not preposition drug delivery sites so as to best target the nidi or origins of chronic medical conditions. The goals in such positioning or prepositioning are procedural optimization and durability. In continued postprocedural treatment, the aims are immediacy and efficacy of response—to lie in wait for and counteract the disorder or disease while nascent through a direct multiply resourced attack to overwhelm and obliterate it in a targeted manner with exposure of nontargeted tissue to the drugs employed eliminated.

Such an implanted system can be prescribed on the basis of a genetic analysis at birth to counteract a predictable or highly probable disorder well before the appearance of symptoms and the condition has the opportunity to advance from the subclinical to the clinical. Ideally, the disorder or disease is counteracted before the patient even becomes aware of it. Such an automatic response system can be placed to treat any existing condition and can be supplemented and reprogrammed as necessary to deal with an additional or a different condition with little more than negligible dissection required.

Placed to dispel an inborn error of metabolism, or another internal medical disorder, or in support of a surgical procedure, the system can be updated to deal with any change in patient status and has a place in the treatment of any but relatively simple and straightforward diseases and procedures. Emplaced preoperatively, the system can not only provide postprocedural monitoring and treatment, but as pertains to compound bypass solid organ transplantation and the replacement of irreparably congenitally malformed vasculature in a neonate with inline coupling jacket-connected tie-line prostheses, can administer the procedure. Where hard wires are best avoided, electrical sensory and command signals can be communicated by wireless transmission and energy transfer to component-inmate batteries recharged by transcutaneous energy transfer.

This also allows for growth from infancy to adulthood. To extend with growth, fluid pipelines can be fluted or configured much as accordion bellows, elastic, and coiled, for example. The means for accomplishing these applications have been described and illustrated in copending application Ser. No. 16/873,914, entitled Vascular Valves and Servovalves—and Prosthetic Disorder Response Systems. In time, failure to back up a more complex operative procedure susceptible to any of a number of adverse sequelae with a totally implanted automatic ambulatory prosthetic disorder response system will be a halfway measure.

Not simple, for example, is diabetes, and the latest means for its treatment to include continuous glucose monitors do nothing to monitor or respond to emergent, or nascent, complications nephropathic, cardiovascular, infective, especially mucocutaneous fungal, neurological, ophthalmic, as well as several others. The most obvious application for such system is one placed to automatically release medication in a prescription nonadherent patient and/or one prevented from optimal medication unless directly pipe-targeted due to a need to avoid serious adverse side effects.

Ideally, symptoms are averted before they appear even when the patient is otherwise engaged, ambulatory, and oblivious. The release is of insulin through a ductus side-entry jacket directly into the portal vein, sensor inputs indicating complications treated as separate arms in the hierarchical control system of which each prompts the release of medication to the respective origin, or root-site or sites of disease, the master controller managing the dispensing of medication to optimize the overall efficacy. In standard use, the term ‘nidus’ is not used to denote a primary site of disease such as a tumor that sheds daughter cells into the circulation to metastasize but is limited to meaning a nest or site from which pathogens emerge to infect distant parts of the body.

Despite being fundamentally inferior to a targeted technique which avoids indiscriminate dispersal and requires minor invasiveness to place, noninvasive is considered the ‘gold standard,’ even in the treatment of serious chronic disease. In this, neither an entirely nor a partially systemic dose is discounted where appropriate. In that it merely relinquishes the use of superior technology to dispel misguided apprehensions, its use when avoidable is actually irresponsible—halfway, indecisive and inconclusive.

The advantages of such a system basic and significant, to refrain from recommending its implementation where appropriate, to instead misrepresent as an enormity the minor surgery required to place it, and persist in prescribing oral medication despite the risk of side effects, or knowingly prescribe less effective medication to avoid the side effects, that is, the conscious use of halfway measures when a more effective response is available can be achieved through the physical targeting of each drug accedes to malpractice. Halfway measures rooted in unjustified hesitancy will result in a materially inferior outcome and quality of life.

Ultimately, there is no serious disease or disorder that would not materially benefit from the constant surveillance and immediate response of such a system, readily implantable in any patient regardless of age, mental competency, or the ability and inclination to adhere to a prescription. By the same token, with surgery as with drugs, the least that works is best. The relatively minor surgery involved in implanting an automatic diagnostic and therapeutic disorder response system is not justification to leave a patient who would benefit from its placement without this protection, failure to do so feckless.

In the 1950s, pediatric cardiac surgery could aspire to no more than sustain the life of the baby. A genuine repair through heart transplantation precluded by the certainty of rejection, only inadequate repair sufficient to keep the baby from dying but not developing normally was possible. The emergence of immunosuppressives represented a major step forward.

The advent of heart transplantation materially improved matters, in that the incorporation of a normal heart made possible the normal development of the child, which attempts at surgical repair and/or the use of mechanical assist devices still cannot achieve. However, the lack of adequate surgical technique, of less traumatizing means for harvesting, preserving, and transplanting the donor heart without effectively strangling and severely wounding, then severely traumatizing the recipient heart to merge the two would critically impair the durability of the operation.

Transplantation still a halfway measure, often the baby would survive for decade, maybe a dozen or so sick and unpromising years and then die anyway, a fate likely facilitated by the impairment inflicted both by the technique employed at the outset as well as weakened immunity resulting from the indiscriminate dispersal of immunosuppressives throughout the systemic circulation rather than by graduating the concentration of and distributing the drugs for optimal effectiveness. Today, to repair a congenitally severely malformed heart with the object of accomplishing no more than to save the life of the baby despite the substandard and shortened life to follow is a ruinous halfway measure not to be tolerated.

Given the trauma to both donor and recipient organs—harvesting with extensive incision and anoxia, then the press of the immune system to eradicate the graft organ, for the average heart transplant to survive for a decade and sometimes longer rates as a welcome but decidedly counterintuitive outcome. Viewed from this perspective, solid organ transplantation as practiced today still represents a halfway measure. The severely congenitally malformed heart still poses a choice between either of two halfway measures—surgical repair that avoids the equal if not greater trauma of transplantation with the risk of rejection but is unable to initiate normal pulsatile, or pulsatant circulation, or a heart transplant that poses the constant threat of rejection.

Pediatric cardiac surgery allows relatively minor to moderate malformities—mostly interventricular defects—to be repaired. However, the complete and sufficient repair of complex defects such as a univentricular heart remains elusive and is not likely ever to become possible. Without normal circulation, all tissues and organs in the body will fail to achieve normal development. The deficits following an optimal repair may be less than conspicuous, and a transplant meticulously supported using conventional means should last for years.

Nevertheless, in either case, the life to follow is likely to be sick and relatively short. Performed as a bridge to heart transplantation, procedures such as the bidirectional Glenn, the hemi-Fontan, Norwood, and fenestrated Fontan are life-sustaining. More significantly, as an end treatment, these do not provide normal circulation and therefore, do not allow normal development. To then leave the patient over a longer term than necessary without a transplant despite the fact that the consequence of impaired circulation is maldevelopment of the body as a whole is a halfway measure when means exist to provide a proper and durable repair.

‘Halfway measure’ is also an appropriate characterization of the current means for the repair of severe congenital malformities of the thoracic aorta and by extension, the aorta in its entirety. Here the situation is much like that of the severely malformed heart—there are numerous inadequate techniques and prostheses, but there is no good and durable repair for such a defect. The only way to fix the aorta once and for all is to replace it or the defective segment with a durable prosthesis that will not dehisce at its junctions, migrate, or leak, will propagate the pulse, and will grow with the patient.

Whether to support a relatively minor repair of the aorta following conventional repair or the replacement of the aorta or a segment thereof with a prosthesis of the kind described in copending application Ser. No. 16/873,914, entitled Vascular Valves and Servovalves—and Prosthetic Disorder Response Systems. Vascular Valves and Servovalves—and Prosthetic Disorder Response Systems filed on 11 Aug. 2020, the placement of lines to deliver medication directly into the repaired aorta or substances to simulate endothelial function in the prosthesis is an improvement upon convention.

Even if there is no frank coarctation or interruption, the baby born with a connective tissue disorder of a severity conducive to the development of an aneurysm should be alleviated of this life-long threat once and for all at the outset. Much as the advent of immunosuppressives rendered heart transplantation feasible leaving the inadequate repair of the severely malformed neonatal heart a halfway measure, the advent of means for the replacement or repair of the carotids with the placement of a pipeline to directly and automatically deliver a topical medication such as a statin into either carotid, leaves a conventional endarterectomy needlessly risky and a halfway measure.

Unlike the carotids in an adult, which congenitally functional have usually become impaired due to the buildup of plaque, the thoracic aorta congenitally malformed to the extent that it cannot be dependably repaired once and for all is not simply degraded and restorable to a previously normal condition. Rather repair in this case is by replacement with a strong and pulse propagating prosthesis sufficiently expandable to accommodate growth from infancy to adulthood.

Copending application Ser. No. 16/873,914, entitled Vascular Vales and Servovalves—and Prosthetic Disorder Response Systems, describes a fabric devised to accommodate growth in vascular prostheses to replace the great or smaller vessels of a tensile strength that eliminates the possibility of a failure in strength equivalent to an aneurysm. As delineated in that application, these are tie-lines connected to the ends of the native vessel at either end by inline coupling jackets which semiautomatically replace a segment along or the entirety of a vessel without the need for clamping the blood supply. While tissue engineering should eventually provide such vessels, current efforts have not produced any that support endothelial function and growth.

For use in an infant, the ability to expand with rapid growth all the way to adulthood is crucial to eradicate the need for numerous reoperations and the iterative draining this inflicts. Electrical conductors can also be conformed for considerable extension, infrequent if any limitations thereto overcome through the use of carrier frequency distinguished wireless reception incorporated into the end connectors or effectors. While completely normal endothelial function is not imparted, function to the extent of simulating the secretion of vasodilators and vasoconstrictors in step with the data provided by prosthetic chemo- and vasopressor sensors is easily accomplished by direct pipeline release into the prosthesis.

Whereas the carotids require a repair in the form of an endarterectomy and rarely replacement, a thoracic aorta congenitally malformed to the extent that is cannot be adequately repaired requires replacement. However, owing to the current state of the art, it cannot be replaced by a strong, expandable, and pulse-responsive prosthesis with branches but must instead be reconstructed or repaired, such as through a combination of a proximal prosthesis and distal intraluminal or endovascular prosthesis as in an ‘elephant trunk’ repair. The fabric described in copending application Ser. No. 16/873,914 solves this problem.

Repair or replacement of the thoracic aorta seldom if ever affords the opportunity to retain the native aortic bodies. The carotids are not more important for detecting hypoxia or hypercapnia and sustaining ventilatory drive to dispel these than is the aortic bodies; however, albeit seldom, the thoracic aorta is subject to much more extensive congenital malformities which demand extensive reconstruction that is denervating and destroys the chemo- and baroreception of the carotid bodies, so that the severely constricted or aneurysmal thoracic aorta, especially when likely to reaneurysm, is best replaced with a prosthesis.

In comparison, the carotids are rarely severely malformed, requiring instead the removal of acquired atherosclerotic plaque in adults. Repairable through a carotid endarterectomy, the unitlateral preservation of the carotid bodies is adapted to more readily than is the loss of the aortic bodies. Moreover, repair rather than the removal and replacement or extensive repair of the carotids allows switching bypass to facilitate the endarterectomy as well as its healing and followup monitoring and therapy.

When an ability to switch from the native structure to a permanent part time bypass prosthesis—as can be provided for the carotids—cannot be provided, as following replacement of the thoracic aorta, the prosthesis nevertheless incorporates at least one drug delivery line, or service or accessory channel, to allow the controller to command the direct delivery into the prosthesis of medication responsive to the need therefor as indicated by the sensors also incorporated into the prosthesis. A prosthetic disorder response system can provide drug delivery lines and the circuitry to govern their use under any circumstances, from the need to counteract an inborn error of metabolism, to the site of a conventional surgical procedure, to the site of procedures and devices which the response system made possible.

Since replacement does not require the ability to switch between the native and prosthetic passageways, the aorta is replaced with nonswitchable inline coupling jackets as the end connectors of the prosthesis to the native stumps, whereas repairable without excision of the carotid bodies, the carotids are repaired with the aid of a bypass that necessitates the use of switchable valves.

That is, both the aortic and carotid procedures consist of implanting a prosthesis, but in the case of the aorta, nonswitchable end connectors are used where in the case of the carotids, switchable end connecting valves are used. Both the switchable bypass type prosthesis connected with valves and the nonswitchable permanent replacement prosthesis connected with inline coupling jackets shown in copending application Ser. No. 16/873,914, entitled Vascular Valves and Servovalves—and Prosthetic Disorder Response Systems, FIGS. 25A and 25B therein, eliminate the use of a shunt and the possibility for an interruption in the flow of blood.

The factors that govern justification for providing an implanted anastomosis flow-through or flow-around switchable bypass—also illustrated in copending application Ser. No. 16/873,914, FIGS. 22B and 32F and the cost thereof with respect to a given repair depend chiefly upon the potential consequences for the organ which could arise due to a problem with the anastomoses:

1. The facilitated healing of the anastomoses when afforded the benefit of flow switched to a bypass.

2. The odds of anastomotic failure, requiring automatically activated ‘bailout’ backup passageways.

3 The odds of partial organ failure due to known deficits of the anastomoses as necessitate a backup capable of compensating for the shortfall.

4. The consequences of a partial or complete failure, to include the need for retransplantation or death.

A compound bypass type heterotopic double heart transplant may be thought of as a switchable bypass.

While refusal to settle for halfway measures once superior measures—such as for replacing the defective thoracic aorta—all of it if necessary—have become available is incontestable, the reciprocal thereof—never replacing a native organ that can be repaired to satisfaction—such as the carotids—is also valid. Means for the semiautomatic replacement of any segment along or the entirety of the aorta or any other larger vessel—consisting of inline coupling jacket substrate vessel end connectors and an expandable span connecting the end connectors—are described and illustrated in copending continuation in part application entitled Vascular Valves and Servovalves—and Prosthetic Disorder Response Systems.

More generally, a genetic analysis of the neonate if not the fetus indicative of any significant metabolic defect or defects should be evaluated for correction first through gene therapy, and if appropriate, the emplacement of an automatic response system to counteract the condition or conditions indicated using gene or other means for elimination or suppression of the condition before it emerges. However, unlike the heart for which no satisfactory artificial replacement that would continue to perform dependably over the life of even an elderly patient exists, to serve in the relatively passive role of an artery, it is possible to provide a durable prosthesis and to do so without the need to interrupt the circulation in order to insert the prosthesis.

Unlike a transplant, a prosthesis poses no risk of provoking an immune response greater than a readily suppressed foreign body reaction. Moreover, a prosthesis is unsusceptible to infection, and requires no blood supply. Much like heart transplantation in the 1950s in having to await the advent of immunosuppressives to progress to basic sufficiency—but still demanding fundamental improvements even today—another procedure that awaits major improvement before it accedes to maturity is carotid endarterectomy. These procedures, transplantation, essential in the treatment of end stage heart failure as well as to save the lives of neonates with severe malformities of the heart from a sick and short life, and an endarterectomy to clear the carotids of atheromatous plaque, are among those needed most frequently.

A conventional carotid endarterectomy risks the escape of thromboembolic debris, and no more than a momentary interruption in the throughflow of blood can result in anoxia, both eventualities posing the risk of stroke. In FIGS. 25A and 25B in copending application Ser. No. 16/873,914, entitled Vascular Valves and Servovalves—and Prosthetic Disorder Response Systems, to avoid the visual confusion that would result were the prosthesis realistically shown as overlying the native vessels, the common, internal, and external carotid components of the prosthesis have been pulled aside to the left. In contrast with this risk entailed in a conventional carotid endarterectomy, neither the three-armed prosthesis described in copending application Ser. No. 16/873,914 using inline coupling jackets at the end of each arm to replace a severely diseased, possibly carotid body-malignant carotid with a Y-shaped prosthesis, nor that using valves or servovalves to bypass the carotids during and after endarterectomy shown in FIGS. 25A and 25B therein risk either of these threats.

The nonvalved device has inline coupling jackets at the end of each arm of the Y-shaped prosthesis and at least one service or accessory channel connected to the internal carotid arm, service or accessory channels to the common carotid and external carotid arms provided according to the overall condition of the patient. The nonvalved device is used to accomplish the replacement of the native carotid by both severing the native structure at each of its three ends and rotating the Y-shaped prosthesis into position a single step. With either the nonvalved or the valved device, bypassed blood flow is closed off from any detritus, and the flow of blood is never interrupted. The valved, or switchable device is used when the native carotids, as in a routine endarterectomy, are preserved with the carotid bodies intact, making the ability to automatically switch back to the native structure when the patient undergoes exertion beneficial.

The direct targeting of maintenance solutions into the prosthesis prevents the accumulation of thrombus or debris along the inner walls and the direct delivery into the internal carotid of medication such as anticonvulsive, antipsychotic, anxiolytic, and so on to the brain, passage through the blood brain barrier possibly necessitating the addition of mannitol in inverse proportion to the age of the patient. Lithium to treat bipolar disorder is kept from the kidneys. Drugs that require conversion in the liver are administered in their post-liver passage active metabolized form. Most surgical procedures performed on the carotids are not to remove these but only to remove plaque, so that the carotid bodies are retained.

The three-armed carotid bypass device with valve, that is, switchable connectors described and shown in FIGS. 25A and 25B in copending application Ser. No. 16/873,914 allows endarterectomy with bypass and retention of the native carotids and carotid bodies with no interruption in throughflow, after which the device can be removed, or left in place as a prosthesis. Left in place, switching between the repaired native carotids and the prosthesis provides several advantages, to include access to the respiratory function of the carotid bodies on an as needed basis, while bypassing blood flow around the native structures during healing, diagnosis, and therapy.

The need for bilateral removal of the carotid bodies is dangerous in eliminating ventilatory drive in response to hypoxemia (see, for example, Wasserman, K. 1978. “The Carotid Bodies: Pathologic or Physiologic?,” Chest 73(5):564-566) resulting in the loss of consciousness and the possibility of serious trauma due to falling, carotid body tumors, usually extra-adrenal paragangliomas, are rare, those bilateral rarer, and those malignant rarer still; however, for those with the bilateral loss of the carotid bodies, their condition is more than sufficiently disabling to demand attention.

Replacement of the native carotids is with the nonvalved, inline coupling jacket end-connected, prosthesis, not the native-to-bypass and bypass-to-native switchable valved device, of which the special value is lost when the native carotids have been removed. In a unilateral removal, the nonvalved device is used on the side of the removal, and where the contralateral carotid is endarterectomized, for example, the valved device is best left in place to allow switching.

Postoperative bypassing with a valved embodiment relieves the healing carotids from needless stress, clears these for treatment or diagnosis, and allows the control microcontroller when signaled by a hypoxemia or hypercapnia sensor to automatically switch to the native carotids to stimulate ventilatory drive, averting syncope and the threat of injury. Fortunately, unlike the bilateral loss of both carotid bodies, the need to remove a carotid is more often unilateral. A bilateral carotid endarterectomy to remove plaque leaves the carotids and the carotid bodies in place, and the loss or extensive reconstruction of the aorta which results in its denervation and loss of the aortic bodies all allow for adaptation and compensation over time.

Nevertheless, certain conditions make it necessary to excise bilateral carotid body tumors resulting in the loss of ventilatory drive responsive to hypoxemia (see, for example, Chen, Y., Li, Y., Liu, J., and Yang, L. 2020. “The Clinical Characteristics and Outcomes of Carotid Body Tumors in Chinese Patients—A STROBE [STrengthening the Reporting of OBservational studies in Epidemiology]-compliant Observational Study,” Medicine (Baltimore, Md.) 99(3):e18824; Butt, N., Baek, W. K., Lachkar, S., Iwanaga, J., Mian, A., and 5 others 2019. “The Carotid Body and Associated Tumors: Updated Review with Clinical/Surgical Significance,” British Journal of Neurosurgery 33(5):500-503; Lin, B., Yang, H., Yang, H., and Shen, S. 2019. “Bilateral Malignant Paragangliomas in a Patient: A Rare Case Report,” World Neurosurgery S1878-S8750(18)32954-1; Hoang, V. T., Trinh, C. T., Lai, T. A. K., Doan, D. T., and Tran, T. T. T. 2019. “Carotid Body Tumor: A Case Report and Literature Review,” Journal of Radiology Case Reports 13(8):19-30; Khurana, A., Mei, L., Faber, A. C., Smith, S. C., and Boikos, S. A 2019. “Paragangliomas in Carney-Stratakis Syndrome,” Hormone and Metabolic Research 51(7):437-442; Anand, J. and Singh, J. P. 2018. “Bilateral Sporadic Carotid Body Tumors—A Rare Case Report,” Radiology Case Reports 13(5):988-992; Burgess, A., Calderon, M., Jafif-Cojab, M., Jorge, D., and Balanza, R. 2017. “Bilateral Carotid Body Tumor Resection in a Female Patient,” International Journal of Surgery Case Reports 41:387-391; Ghali, M. G. Z., Srinivasan, V. M., Hanna, E., and DeMonte, M. 2017. “Overt and Subclinical Baroreflex Dysfunction after Bilateral Carotid Body Tumor Resection: Pathophysiology, Diagnosis, and Implications for Management,” World Neurosurgery 101:559-567; Han, L. V., Chen, X., Zhou, S., Cui, S., Bai, Y., and Wang, Z. 2016. “Imaging Findings of Malignant Bilateral Carotid Body Tumors: A Case Report and Review of the Literature,” Oncology Letters 11(4):2457-2462; Nicholas, R. S., Quddus, A., Topham, C., and Baker, D. 2015. “Resection of a Large Carotid Paraganglioma in Carney-Stratakis Syndrome: A Multidisciplinary Feat,” British Medical Journal Case Reports 2015:bcr2014208271; Rosa, M. and Sahoo, S. 2008. “Bilateral Carotid Body Tumor: The Role of Fine-needle Aspiration Biopsy in the Preoperative Diagnosis,” Diagnostic Cytopathology 36(3):178-180).

In a prosthetic disorder response system where the carotids with carotid bodies had to be removed, hypoxemia or hypercapnia would be readily detected by one or more tiny implanted pulse oximeter sensors, for example, from which low value inputs would signal the controller to directly electrostimulate the breathing centers in the medulla and pons.

System Control of Multidrug Delivery Systems

According to the present concept, a pharmacist-programmer enters this into a program whereby each drug is provided in response to the conditions sensed. To deliver drugs automatically and adjust the dosing, the prescription, an adaptive drug delivery program, responds to diagnostic sensor feedback under the control of a medically adapted hierarchical (nodal, nested-levels) ‘intelligent’ hard real-time ‘pathfinding’ control system (references on hierarchical control are provided below).

Less complex than is comorbid, much less multimorbid disease that necessitates a divide-and-conquer approach, monomorbid disease will usually not require multiple level, or hierarchical, control administered by an implanted microprocessor serving as the master controller that integrates the pre-processed data of subordinate nodes or controllers and issues drug release commands. In a comorbid diagnostic and therapeutic system, microcontrollers descend from the level of a monomorbid master node to a node subordinate to the master microprocessor.

Automatic ambulatory disorder response systems to monitor, diagnose, and treat relatively straightforward monomorbid disease and the nodes or controllers subordinate to the master control microprocessor in a hierarchical control system are usually highly miniaturized, large scale integrated single chip microcontrollers such as those produced by Microchip Technology's PIC [Peripheral (or Programmable, Interface Controller or Programmable Intelligent Computer] line and Atmel, for example. For implantation, these are housed to provide thermal insulation and a chemical barrier to prevent contact with tissues.

Since microcontroller and multicore microcontroller input pins are needed to set the program, additional pins to input collateral functions such as those from sensors placed to signal changes in medical conditions and outputs to execute the program, and a significant storage capacity needed to record potential changes, the microcontroller assigned to any given drug reservoir outlet pump-pair plug-in pump-pack such as those depicted in copending application Ser. No. 14/121,365, entitled Ductus Side-entry Jackets and Prosthetic Disorder Response Systems, FIGS. 29, 31, 32, and 36 therein or the implanted equivalent thereof in a distributed set of pump-packs under unified control must provide a number of pins and performance capacity consistent with industrial multicore microcontrollers.

The PICoPLC program ladder logic editing, simulating, and compiling tool can generate native code for 8-bit and 32-bit microcontrollers, such as the Parallax, Inc. Propeller and Microchip Technology PIC16 central processing units from a ladder diagram, effectively gaining in a microcontroller a level of integrative capability associated with programmable logic controllers (see, for example, M. Rafiquzzaman 2018. Microcontroller Theory and Applications with the PIC18F; New York, N.Y.: Wiley; Haddad, N. K. 2017. Microcontroller System Design Using PIC18F Processors, Jacksonville, Fla.: IGI Global; Dogan Ibrahim 2014, PIC Microcontroller Projects in C: Basic to Advanced (for PIC18F), London, England: George Newnes Limited;. Sanchez, J. P and Canton, M. P 2006. Microcontroller Programming: The Microchip PIC, Boca Raton, Fla.: Chemical Rubber Company Press; Iovine, J. 2000. PIC Microcontroller Project Book, New York, N.Y.: TAB [Technical Author's Bureau] Books Publishing Company.

For these and other microcontrollers, further reduction in size and power consumption are afforded through discretization, whereby the continuous steam of data is converted into a sequence of data points with sufficient accuracy preserved for control purposes. Sensor inputs that justify proportional-integral-derivative closed loop feedback from implanted sensors may be discretized.

Conversion of closed loop physiological or life-sign input data into a sequence of points then overcomes the need for an expensive and larger programmable logic controller able to perform the ongoing calculation essential to control the continuous process as such (see, for example, Uzunovic, T. and Turkovic, I. 2012. “Implementation of Microcontroller Based Fuzzy Controller,” 6th Institute of Electrical and Electronics Engineers International Conference on Intelligent Systems, Sofia, Bulgaria, available at Institute of Electrical and Electronics Engineersxplore. Institute of Electrical and Electronics Engineers.org; Velagic, J., Kuric, M., Dragolj, E., Ajanovic, Z., and Osmic, N. 2012. “Microcontroller Based Fuzzy-PI [Proportional-Integral] Approach Employing Control Surface Discretization,” 20th Mediterranean Conference on Control and Automation, Barcelona, Spain, available at Institute of Electrical and Electronics Engineersxplore.Institute of Electrical and Electronics Engineers.org; Avery, S., Gracey, C., Graner, V., Hebel, M., Hintze, J., LaMothe, A., Lindsay, A., Martin, J., and Sander, H. 2010. Programming and Customizing the Multicore Propeller Microcontroller: The Official Guide, New York, N.Y.: McGraw-Hill; Nass, M. 2010. “Xilinx Puts ARM [advanced reduced instruction set computation machine] Core into its FPGAs [field-programmable gate arrays],” Embedded, available at http://www.embedded.com/electronics-products/electronic-product-reviews/embedded-tools/4115523/Xilinx-puts-ARM-core-into-its-FPGAs; McConnel, T. 2010. “ESC—Xilinx Extensible Processing Platform Combines Best of Serial and Parallel Processing,” Electronic Engineering Times, available at http://www.eetimes.com/document.asp?doc_id=1313958; Cheung, K. 2010. “Xilinx Extensible Processing Platform for Embedded Systems,” available at http://fpgablog.com/posts/arm-cortex-mpcore/; Kanagaraj, N., Sivashanmugam, P., and Paramasivam, S. 2009. “A Fuzzy Logic based Supervisory Hierarchical Control Scheme for Real Time Pressure Control,” International Journal of Automation and Computing 6(1):88-96; Keckler, S. W., Olukotun, K., and Hofstee, H. P. 2009. Multicore Processors and Systems, New York, N.Y.: Springer; Scanlan, D. A. and Hebel, M. A. 2007. “Programming the Eight-core Propeller Chip,” Journal of Computing Sciences in Colleges 23(1):162-168). Linear stage motors usually steppers, other type motors are not to be excluded.

When used for the direct pipeline-targeted delivery of drugs into vessels through side-entry jackets or into a volume of tissue by nonjacketing side-entry connectors (references cited above under Cross Reference to Related Applications), a primary object in the use of and is to implement drug delivery aligned to network feedback. When the data is complex, it is processed to include data reduction and integration by means of a hierarchical control system.

Where diagnostic data alone would leave it to the diagnostician to translate the data into remedial action drug delivery could not be immediate, pharmacokinetically and pharmacodynamically optimized, nor unerringly targeted, automatic control that breaks down, integrates, and compares the data up through levels that progressively coordinate more encompassing cross morbidity data makes possible diagnosis and therapy that is optimized in each of these regards. If the patient is not to be bedridden or the condition is chronic, a number of needled catheters cannot be used. Ductus side-entry connection jackets afford secure connection to the ductus, and in so doing, enable not just single point direct-to-ductus drug delivery, but the implementation of such a prosthetic supplementary disease-process compensation system.

The side-entry ductus side-entry jackets, nonjacketing side-entry connector, and drug reservoir outlet pump-pair sets to be described thus make possible the targeted delivery of drugs through automatic response that is immediate. Were the condition to exceed the range of adjustment for which the system had been set, the exigent readings can be transmitted to a clinician able to adjust the dosing by remote control.

Sensors that must not be allowed to lose in sensitivity due to the predictable development of a sensor-enveloping fibrous capsule are shielded from this eventuality with an service or accessory channel to deliver a dissoluting drip such as dilute hydrochloric acid or a dilute hypochlorite such as bleach to occasionally wet that part or parts of the sensor outer surface which must be afforded a clear ‘sight line.’ If a minute amount per drip is satisfactory and would serve to reduce the implant load, a centralized reservoir is used to release the dissoluting agent to all sensors that need it. Since the probability is high that the agent will be reacted to as an irritant, the dissolutive agent is alternated with an irritant counteractant.

In the case of hydrochloric acid, the counteractant is sodium bicarbonate. So that implicative data is always identified to its location, and remedial measures, usually drugs, can be immediately targeted to the site or sites, both sites of primary disease, and sites likely to present progressive, sequelary, or an associated continuation of the disease process, disease analyte-detecting sensors such as report a disproportionate concentration in T or B cells or sites of elevated temperature indicative of malignancy are always mapped to the control system. The location of each sensor is included in the output data. With known disease, the sensors chosen should be closely selective for the detailed analytes as earmarks diagnostic for the disease.

In what may be best described as a ‘lying-in-wait’ posture, sensors are positioned where cellular or otherwise low-level symptoms are most likely to appear first. So that the patient need not undergo another endoscopic procedure to position additional sensors to monitor secondary disease predictable ab initio, the additional sensors, drug reservoirs, and pipelines are placed at the outset. Where the odds for the emergence of any one of a number of equally possible sequelary diseases are equal, if possible, broad spectrum analytes diagnostic for either are monitored, blood and urine draws, for example, used to secure a positive identification.

Reduction in the number and volume of drugs supports elimination of the need for the patient to wear of a paracorporeal, usually belt-worn, drug reservoir and pack, whether needed for power, control, or to house pumps. Paracorporeal, or belt-worn packs are discouraged as inviting tinkering and interfering with a fully closed skin implementation. To the extent possible, an automatic ambulatory disorder response system should preserve the outward integrity of a normal body to include freedom from the need to wear mandatory equipage especially an indispensable body pack. In most instances, the number of comorbidities to be addressed and the drugs needed to deal with these will be few enough that the drug reservoirs and/or power requirement will not compel the need for this impediment.

If possible, to allow full, that is, closed-skin implantation, all components of the automatic response system are miniaturized, and implemented with large scale integrated microelectronics. As shown in copending application Ser. No. 16/873,914, entitled Vascular Valves and Servovalves—and Prosthetic Disorder Response Systems, FIGS. 26A and 26B therein, small drug reservoirs are replenished through closed-skin body surface ports by multiple nozzle injection syringes such as that shown in FIG. 27A therein or by multiple nozzle jet injectors such as that shown in FIG. 27B therein.

Each nozzle can deliver the same or a different drug to replenish different reservoirs. A port described in copending application Ser. No. 14/121,365 incorporates means other than a conventional skin button or skin barrier for averting infection and instability. The port provides as many entry holes as the number of drug reservoirs and pipelines feeding into service or accessory channels requiring periodic drug replenishment.

Mechanical rather than electronic embodiments depicted in copending application, Ser. No. 15/998,002, entitled Ductus Side-entry Jackets and Prosthetic Disorder Response Systems FIGS. 29, and 31 thru 36 therein were intended to convey a clearer pictorial representation than might be provided using schematic diagrams and because the system and because implementation was in terms of these components being stored in a power and control pack. In most cases, it is preferable both from the standpoint of freedom from the need to wear a pack and inaccessibility to the inquisitive to use electronic components equivalent to those mechanical shown where the entire disorder response system is inside the body.

With either type device, injection is through a multiple opening mediport- or portacath-type port positioned subcutaneously (subdermally) in the pectoral region without an opening to the outside. Such a port allows the replenishment of drugs through the skin and a self-resealing cover membrane through which the injection needle or needles are passed. Tiny tattooed dots overlying the subdermal port indicate how the injector is to be aligned to assure the release of each drug into the proper reservoir.

A body surface port with an opening to the outside is used only when miniature cabled devices to include angioscopes, excimer lasers, and various diagnostic probes, chemical and imaging, diagnostic and/or therapeutic, are to be freely pass through to the origin, or root-site of disease from outside the body, or when placed to a side of the mons pubis, to allow the passage of urine into a collection bag. Prostheses that bypass the lower urinary tract are fully described and illustrated in copending application Ser. No. 16/873,914, entitled Vascular Valves and Servovalves—and Prosthetic Disorder Response Systems. Broadly, cautious diagnosis and drug selection in preparation for system placement best fosters system efficiency.

In a prosthetic disorder response system placed to optimize the overall health of a patient with multiple comorbidities, the urinary tract—or in the absence of a urinary tract, equivalent removal of toxins from the blood of nocuous substances—is represented as one of the channels of control in a hierarchical control system. To optimize magnetic separation, or extraction, the electromagnets are made as light in weight as enclosure within a thin but tough nonallergenic plastic case and windings of silver wire, which provide greater field strength for the weight, will allow. If necessary, the micro- or nanoparticle carrier particles bonded to the target analyte or analytes are formulated to incorporate silicon-iron crystal, materially increasing their magnetic susceptibility.

Ancillary factors as pertain to specific pharmaceuticals, tissue expansion to create an intracorporeal pocket to hold one in or more drug reservoirs or another component of the implanted drug distribution system, the use of different electric current discharge patterns from the semicircular anchoring needles of nonjacketing tissue connectors in combination with different drugs passed through the drug pipeline connected to the substrate tissue by the nonjacketing side-entry connectors, and numerous other related topics, are covered in Nonprovisional application Ser. No. 14/998,495, entitled Nonjacketing Side-entry Connectors and Prosthetic Disorder Response Systems filed on 12 Jan. 2016, and Nonprovisional application Ser. No. 15/998,002 entitled Ductus Side-entry Jackets and Prosthetic Disorder Response Systems, both filed under 35 U.S.C. 1 19(e) on 25 Aug. 2014.

For medical use, such jackets and nonjacketing connectors must remain leak-free, nonmigrating or nondislodgeable, nondeformable, nonfracturing, and not injurious to the substrate or neighboring tissue indefinitely. Moreover, for pediatric use, these must adapt to growth over a period of years. Copending application Ser. No. 14/121,365 also addressed means for securely fastening catheteric lines, injection needles, and electrodes, for example, to native ductus through a small entry wound for the long-term treatment of chronic conditions, and delineated the assignment of channels or axes of control in a hierarchical control system to different organs or organ systems in the treatment of comorbid disease, for example.

Detailed information on the structure and function of the different type connectors used in a prosthetic disorder response system will be found in the copending applications specified. The foregoing applications also provide detailed information on the treatment of many diseases and methods of treatment, to include nephrogenic systemic fibrosis, diabetic gastroparesis, solid tumors benign and malignant, the direct and point-blank targeting of tumors internal to delicate organs that to excise surgically would inflict an inordinate degree of trauma, the different types of radiation shielding, to include Auger and higher dose rate radioisotopes used to isolate drug pipelines conducting substances at a dose rate than would be contained by the material of the pipeline itself.

Also addressed are the use of such a control system to alleviate the symptoms of urinary dysfunction such as urethra-noncompressive reinstatement of urinary continence, Vineberg-derived prevention of hypoxia and reperfusion in different contexts, such as venous stasis ulcers of the lower leg, targeted interdiction of a cirrhosis-inducing cascade, stereotactic drug steering by magnetic vectoring, discrete point, and point-to-point through-tissue, transmission, measurement, and telemetry, and targeted electrical and/or chemical autonomic motor assistance, in addition to numerous related topics.

Wireless body area networks with wireless transmission or telemetry is addressed with references provided in copending application Ser. No. 14/121,365, entitled Ductus Side-entry Jackets and Prosthetic Disorder Response Systems, filed on 25 Aug. 2014. The automatic drug selection and delivery control program or prescription data switches the drug reservoir catheters connected to each target ductus from among an unlimited number of drug supply reservoirs. In this, a body area network under simple or intelligent’ complex, meaning hierarchical adaptive control, can be connected to transmit data through a wireless network.

### Concept of the Invention

The invention relates first to

1. A fully implanted system for the segregated and isolated delivery of drugs to the sites of disease which eliminates several constraints that have limited drug efficacy in the past, and second, to:

2. Diagnostics and therapeutics which due to the direct access imparted by internal prepositioning that eliminates the need to reenter, can be directed remotely and/or automatically; and third, to:

3. An automated system for determining the identity, apportionment, and dosing best suited to each in a plurality of morbidities and for the combination thereof and for effectuating the directly pipeline-targeted delivery of the drugs to the targets so that the treatment will have been optimized for each morbidity as well as for the sum thereof.

4. In marked contrast to superficially similar apparatus such as indwelling catheters and continuous glucose monitors, the system is devised to remain functional around the clock, while the patient is ambulatory and likely unaware of its presence, and to do so throughout life, in some cases, even when initially implanted in a neonate.

To this end, copending application Ser. No. 16/873,914 describes a fabric great in tensile strength, securely fixed in position—such as along the thoracic aorta to replace a congenital aneurysm, coarctation, or interrupted aortic arch incapable of satisfactory repair—by means of inline coupling jackets at either end, and capable of expansion sufficient to accommodate growth from the neonate to the adolescent, if not the adult. The importance of avoiding an initial repair lacking sufficient durability to allow strenuous exercise and incapable of expansion, and the need this affirms for multiple revisions in a child weak to begin with speaks for itself.

The two greatest impediments in the use of drugs are the risk of adverse side effects and patient nonadherence to the prescription regimen. Both are eliminated, the first through isolated delivery, the second by automatic release. With an object to provide such systems in a form that will remain fully dependahle for many years if not life, allied applications identified above described highly durable ductus and tissue connectors that would not injure underlying or neighboring tissue.

Described here are fully, or closed-skin, implanted control systems that seek to overcome both of these disadvantages. Side effects arise when a drug introduced into the circulation comes into contact with tissue other than that intended. A familiar instance is the exposure to oncologic drugs which mitocidal, target the quickly replicating cells of a malignancy but also those which produce hair and gametes. Other examples include increased vulnerability to infection in patients prescribed immunosuppressives and the moon facies associated with steroids. All drugs have side effects, and the medical benefits of limiting dosage levels to those which will not harm unintended organs, glands, or tissues while producing the optimal effect in that intended pertains to all drugs.

Copending application Ser. No. 13/694,835 addresses the targeting of radiopharmaceuticals not on the basis of an inherent metabolic affinity of the target organ such that of the thyroid gland for iodine, but rather through the application of magnetic attractive force to superparamagnetic such as magnetite or maghemite drug carrier nanoparticles to which the radiopharmaceutical is bound within a ferrofluid introduced into the pre- or post-heapatic systemic circulation rather than delivered directly to the target (see, for example, Wilfried Andra, W. and Speer, T. 2010. Targeted Radionuclide Therapy, Lippincott Williams & Wilkins; Nowak, H. (eds.) 2006. Magnetism in Medicine: A Handbook, Hoboken, N.J.: John Wiley and Sons).

In copending application Ser. No. 16/873,914, entitled Vascular Valves and Servovalves—and Prosthetic Disorder Response Systems is described the use of such systems to administer surgical procedures, to include solid organ transplantation by means of a compound bypass procedure that switches the circulation of the recipient from his own native to the donor organ, and if appropriate, allows the donor organ to be harvested at the around the midway points in this switch, the donor organ then implanted as a second heart or a supernumerary thyroid gland, for example, where the native organ had grown sufficiently incompetent to justify placement of a coworker as an assist device.

Such a system can also administer an extracardiac reversal of a transposition of the great vessels, the semiautomatic removal and replacement with a prosthesis of any segment along a ductus in any length with its branches, to include the aorta or main pulmonary vessels, for example, in no case requiring a state of circulatory arrest. Some tissues have an intrinsic predilection for a particular agent moving through the circulation. The affinity for iodine of the thyroid gland makes it possible to chemically target the thyroid through the circulation without the need to literally isolate the iodine in a mechanical sense by direct pipeline delivery.

Following an organ transplant, the same system that administered the compound bypass transfer procedure will assure that immunosuppressives are released and optimally directed in perpetuity, thus eliminating deviation from the prescription regimen as a key deterrent in organ transplantation, especially in the very old, the very young, the very sick, the psychotic, and those with impaired cognitive ability. But the affinity of iodine for the thyroid gland is exceptional.

Examples of drugs effective in treating the target while causing grave damage elsewhere in the body are anticholinergic and antimuscarinic negative inotropes used to subdue the excitable neurons of the detrusor muscle of which this hyperactivity causes overactive bladder and frequent urination. Little if at all problematic when used earlier in life, once the blood brain barrier begins to break down with age, these drugs progressively gain greater access to the neurons of the brain where they bring about and cooperate in bringing about dementia.

Just as drugs should be prescribed to include the fewest in the smallest doses as possible, except where disease is progressive or sequelary, the implanted system should include only the fewest components in the simplest embodiments that will accomplish the function desired. In comorbid disease, components that cease to be necessary are generally not explanted for a time to allow reassignment to another function.

The release into the circulation of negative inotropes poses the risk of adverse effects on nervous function throughout the body. For example, peristalsis may be slowed resulting in constipation, and nervous control over the heart can induce dysrhythmias. The direct targeting to the detrusor of botulinum toxin, for example, minimizes if not eliminates contact with the toxin of other tissue to include a dysrhythmic heart still suspected to be dysynchronized as a possible result (see, for example, Miotla, P., Olejnicza, P., Futyma, K., Wrobel, A., Tomaszewski, M., and 4 others 2018. “Can Intradetrusor OnabotulinumtoxinA Injections Alter Heart Function in Patients with Cardiac Arrhythmia?,” Journal of Clinical Medicine 7(9):263; online at https://doi.org/10.3390/jcm7090263).

Reference here to disorders of the urinary tract are purely exemplary in serving to illustrate the unlimited versatility that includes facilitating and optimizing the application of collateral advancements in treatment provided by fully implanted disorder response systems as described in this and related documents. Such a system can configured to diagnose and treat any problem known to the field of internal medicine, a detailing here not limited to one area such as urological impracticable if not superfluous. Wider applicability is, however, provided in the applications listed above. FIG. 12A in U.S. Pat. No. 11,013,858, reproduced here as FIG. 1, which shows a nonjacketing side-entry connector, part number 61, connected to a side of the upper surface, or dome, of the detrusor, is exemplary in showing one connector; however, several small connectors positioned at different points about the bladder considered an obvious variant of the arrangement shown.

Access to the parenchyma of a solid organ is through a side-entry jacket or valve on the blood supply or as shown here, through a nonjacketing side-entry connector fastened to the adventitia or fibrosa. Provided with hollow anchoring needles, such a connector can periodically inject drugs such as anticholinergic, antimuscarinic, mirabegron, or botulinum toxin, for example, into the detrusor or bladder to dispel bladder hyperactivity without exposure of the brain and risk of dementia in an elderly patient, sodium bicarbonate to neutralize the acidity of urine erosive to the cystic urothelium when already injured, or lidocaine to dipel the pain of interstitial cystitis, for example, with little if any spillover into the systemic circulation. Such treatment can be temporary pending more durable therapy, the latter likely supported by adjuvant medication if not administered through the same connections.

The isolated directly pipeline-targeted delivery into the bladder of anticholinergic drugs, antimuscarinic drugs, and botulinum toxin or electrical stimulation avoids the numerous adverse side effects associated with systemic delivery that adversely affects neurons elsewhere in the body (references provided in copending application Ser. No. 16/873,914, entitled Vascular Valves and Servovalves—and Prosthetic Disorder Response Systems, Section 2a(2)(g)c, entitled Vascular valves and Servovalves in Urinary Diversion

The significant deterrents to the use of mirabegron, antimuscarinis, anticholinergics, and botulinum toxic to quell an overactive bladder are delineated with references in copending application Ser. No. 16/873,914.

Provided with electrical current conducting anchoring needles, nonjacketing side-entry connectors can periodically deliver electrical stimulation of the detrusor, tibial, and sacral nerves with less bulk, and applied in different positions, with better distribution than might a solitary electrical neuromodulator. Separate connectors with electrified needles can deliver intermittent shocks where the discharge from each needle, each combination of needles, and from differently positioned connectors can be different in phase and sequence. Moreover, the electrical discharge and injection functions are independently controllable.

This capability makes it possible to induce a response in the detrusor which can be patterned to simulate and so approximate that induced by low energy, or low intensity, shock wave therapy, disposing of drug-induced side effects altogether. As does excessive shock delivery in the form of electrostimulatory defibrillatory resynchronization of the heartbeat, for example, this capability if not used sparingly can prove injurious and is therefore used as supplementary to treatment with shock wave therapy when the patient is away from the clinic. Moreover, the ameliorative effects of electrical neuromodulation within the bladder can also be combined with the abrupt displacements imposed by an extracorporeal lithotripter to inhibit inflammation and oxidative stress in the chronically hypoxic, potassium metabolism-impaired, and/or leaky urothelial lining of the bladder.

Whether separate or concurrent with extracorporeal shock wave therapy, the direct delivery by the system of mesenchymal stem cells and/or genetically modified stem cells can promote vasodilatation and angiogenesis to further reduce this condition (see, for example, Lu, J.-H., Chueh, K.-S., Chuang, S.-M., Wu, Y.-H., Lin, K.-L. and 5 others 2021. “Low Intensity Extracorporeal Shock Wave Therapy as a Potential Treatment for Overactive Bladder Syndrome, Biology (Basel, Switzerland) 10(6):540; Chuang, Y. C., Meng, E., Chancellor, M., and Kuo, H. C. 2020. “Pain Reduction Realized with Extracorporeal Shock Wave Therapy for the Treatment of Symptoms Associated with Interstitial Cystitis/Bladder Pain Syndrome—A Prospective, Multicenter, Randomized, Double-blind, Placebo-controlled Study,” Neurourology and Urodynamics 39(5):1505-1514; Skaudickas, D., Telksnys, T., Veikutis, V., Aniulis, P., and Jievaltas, M. 2020. “Extracorporeal Shock Wave Therapy for the Treatment of Chronic Pelvic Pain Syndrome,” Open Medicine (Warsaw, Poland). 15(1):580-585; Lander, E. B., Berman, M. H., and See, J. R. 2019. “Personal Cell Therapy for Interstitial Cystitis with Autologous Stromal Vascular Fraction Stem Cells,” Therapeutic Advances in Urology 11:1756287219868590; Sun, D. Z., Abelson, B., Babbar, P., and Damaser, M. S. 2019. “Harnessing the Mesenchymal Stem Cell Secretome for Regenerative Urology,” Nature Reviews. Urology 16(6):363-375; Yuan, P., Ma, D., Zhang, Y., Gao, X., Liu, Z., and 5 others 2019. “Efficacy of Low-intensity Extracorporeal Shock Wave Therapy for the Treatment of Chronic Prostatitis/Chronic Pelvic Pain Syndrome: A Systematic Review and Meta-analysis,” Neurourology and Urodynamics. 38(6):1457-1466; Ryu, C. M., Yu, H. Y., Lee, H. Y., Shin, J. H., Lee, S., and 11 others 2018. “Longitudinal Intravital Imaging of Transplanted Mesenchymal Stem Cells Elucidates Their Functional Integration and Therapeutic Potency in an Animal Model of Interstitial Cystitis/Bladder Pain Syndrome,” Theranostics 8(20):5610-5624; Zhu, G. Q., Jeon, S. H., Bae, W. J., Choi, S. W., Jeong H. C., and 8 others 2018. “Efficient Promotion of Autophagy and Angiogenesis Using Mesenchymal Stem Cell Therapy Enhanced by the Low-energy Shock Waves in the Treatment of Erectile Dysfunction,” Stem Cells International 2018:1302672; Kim, A., Yu, H. Y., Lim, J., Ryu, C. M., Kim, Y. H., and 16 others 2017. “Improved Efficacy and in Vivo Cellular Properties of Human Embryonic Stem Cell Derivative in a Preclinical Model of Bladder Pain Syndrome,” Scientific Reports 7(1):8872; Kim, A., Shin, D. M., and Choo, M. S. 2016. “Stem Cell Therapy for Interstitial Cystitis/Bladder Pain Syndrome,” Current Urology Reports 17(1):1; Lee, J. D. and Lee, M. H. 2011, “Increased Expression of Hypoxia-inducible Factor-1 alpha and Vascular Endothelial Growth Factor Associated with Glomerulation Formation in Patients with Interstitial Cystitis,” Urology 78(4):971.e11—e15; Parsons, C. L. 2011. “The Role of a Leaky Epithelium and Potassium in the Generation of Bladder Symptoms in Interstitial Cystitis/Overactive Bladder, Urethral Syndrome, Prostatitis, and Gynaecological Chronic Pelvic Pain,” British Journal of Urology International 107(3):370-375; Kim, J., Keay, S. K, and Freeman, M. R. 2009. “Heparin-binding Epidermal Growth Factor-like Growth Factor Functionally Antagonizes Interstitial Cystitis Antiproliferative Factor via Mitogen-activated Protein Kinase Pathway Activation,” British Journal of Urology International 103(4):541-546; Parsons, C. L., Greene, R. A., Chung, M., Stanford, E. J., and Singh, G. 2005. “Abnormal Urinary Potassium Metabolism in Patients with Interstitial Cystitis,” Journal of Urology 173(4):1182-1185).

Internalizing the delivery of drugs such as oxybutynin and nitroglycerin by delivery through a side-entry connector in lieu of administration by transdermal patch eliminates a local skin reaction (see, for example, Nitti, V. W. 2003, “Transdermal Therapy for Overactive Bladder: Present and Future,” Reviews in Urology 5 (Supplement 8):531-536). The use of drugs conventionally applied to ameliorate detrusor hyperactivity, stem cells, low energy extracorporeal shock wave therapy, electrostimulation applied in any pattern until that determined optimal is chosen, and the directly pipelined delivery into the detrusor of a vasodilator are compatible, and except for the use of a lithotripter, can be prescription-programmed for the system controller to automatically continue in coordination or independently.

The electrical discharge capability of conductive anchoring needles in nonjacketing side-entry connectors has widespread application in accelerating the rate of wound healing (see, for example, Sebastian, A., Syed, F., Perry, D., Balamurugan, V., Colthurst, J., Chaudhry, I. H., and Bayat, A. 2011. “Acceleration of Cutaneous Healing by Electrical Stimulation: Degenerate Electrical Waveform Down-regulates Inflammation, Up-regulates Angiogenesis and Advances Remodeling in Temporal Punch Biopsies in a Human Volunteer Study,” Wound Repair and Regeneration 19(6):693-708).

The muscular coat (tunica muscularis, detrusor urinae muscle) of the urinary bladder (detrusor urinae) consists of smooth muscle fibers in three layers (see, for example, Gray, H. 28th ed. 1966. Anatomy of the Human Body, Philadelphia, Pa.: Lea and Febiger, Chapter 17, page 1295. A nonjacketing side-entry connector with anchoring needles both hollow and electrically conductive can apply shocks and inject medication such as vasodilatory at any time to include simultaneously, and a ductus side-entry jacket or valve can deliver drugs and other agents into the blood supply of target tissue.

This capability means that used individually or in combination, there is no implementation of these therapeutic modalities individually or in combination which the implanted prosthetic disorder system cannot perform as well as support in an adjuvant sense, and moreover, without human error and therefore greater dependability (see, for example, Gupta, A., Rodriguez, H. C., Delfino, K., Levy, H. J., El-Amin, S. F. 3rd, and Gaines, R. 2021. “Evaluation of Immediate and Short-term Efficacy of DualStim Therapy with and without Intracavernosal Umbilical Cord-derived Wharton's Jelly in Patients with Erectile Dysfunction: Study Protocol for a Randomized Controlled Trial,” Contemporary Clinical Trials Communications 23:100790; Li, G. and Man, L 2021. “Low-intensity Extracorporeal Shock Wave Therapy for Male Chronic Pelvic Pain Syndrome: A Systematic Review and Meta-analysis,” Translational Andrology and Urology 10(3):1202-1211; Lin, K. L, Lu, J. H., Chueh, K. S., Juan. T J., Wu, B. N., and 5 others 2021. “Low-intensity Extracorporeal Shock Wave Therapy Promotes Bladder Regeneration and Improves Overactive Bladder Induced by Ovarian Hormone Deficiency from Rat Animal Model to Human Clinical Trial,” International Journal of Molecular Sciences 27; 22(17):9296; Sandoval-Salinas, C., Saffon, J. P., Corredor, H. A., Fonseca, L., Manrique, L., and Solis, G. 2021. “Are Radial Pressure Waves Effective in Treating Erectile Dysfunction? A Systematic Review of Preclinical and Clinical Studies,” Sexual Medicine 9(4):100393; Shin, D., Jeon, S. H., Tian, W. J., Kwon, E. B., Kim, G. E., and 5 others 2021. “Extracorporeal Shock Wave Therapy Combined with Engineered Mesenchymal Stem Cells Expressing Stromal Cell-derived Factor-1 Can Improve Erectile Dysfunction in Sreptozotocin-induced Diabetic Rats,” Translational Andrology and Urology 10(6):2362-2372. Kim, S. W., Zhu, G. Q., and Bae, W. J. 2020. “Mesenchymal Stem Cells Treatment for Erectile Dysfunction in Diabetic Rats,” Sexual Medicine Reviews 8(1):114-121; Furuta, A., Yamamoto, T., Igarashi, T., Suzuki, Y., Egawa, S., and Yoshimura, N. 2018. “Bladder Wall Injection of Mesenchymal Stem Cells Ameliorates Bladder Inflammation, Overactivity, and Nociception in a Chemically Induced Interstitial Cystitis-like Rat Model,” International Urogynecology Journal 29(11):1615-1622; Jeon, S. H., Zhu, G. Q., Bae, W. J., Choi, S. W., Jeong, H. C., and 9 others 2018. “Engineered Mesenchymal Stem Cells Expressing Stromal Cell-derived Factor-1 Improve Erectile Dysfunction in Streptozotocin-Induced Diabetic Rats,” International Journal of Molecular Sciences 19(12):3730; Wang, B., Zhou, J., Banie, L, Reed-Maldonado, A. B., Ning, H., and 9 others 2018. “Low-intensity Extracorporeal Shock Wave Therapy Promotes Myogenesis through PERK/ATF4 Pathway,” Neurourology and Urodynamics 37(2):699-707; Xie J, Liu B, Chen J, Xu Y, Zhan H, and 3 others 2018. “Umbilical Cord-derived Mesenchymal Stem Cells Alleviated Inflammation and Inhibited Apoptosis in Interstitial Cystitis via_AKT/mTOR [protein kinase B/mammalian (or mechanistic, FK506-binding protein 12-rapamycin-associated protein 1 (FRAP1) target of rapamycin] Signaling Pathway,” Biochemical and Biophysical Research Communications 495(1):546-552; Lu, Z., Lin, G., Reed-Maldonado, A, Wang, C., Lee, Y. C., and Lue, T. F. 2017. “Low-intensity Extracorporeal Shock Wave Treatment Improves Erectile Function: A Systematic Review and Meta-analysis,” European Urology 71(2):223-233; Manger, N., and Türkeri, L. 2017. “Stem Cell Therapies in Post-prostatectomy Erectile Dysfunction: A Critical Review,” Canadian Journal of Urology 24(1):8609-8619; Prieto, C. P., Ortiz M. C., Villanueva A., Villarroel, C., Edwards, S. S. and 6 others 2017. “Netrin-1 Acts as a Non-canonical Angiogenic Factor Produced by Human Wharton's Jelly Mesenchymal Stem Cells (WJ-MSC),” Stem Cell Research and Therapy 8(1):p. 43; Yiou, R., Mahrouf-Yorgov, M., Trébeau, C., Zanaty, M., Lecointe, C., and 4 others 2016. “Delivery of Human Mesenchymal Adipose-derived Stem Cells Restores Multiple Urological Dysfunctions in a Rat Model Mimicking Radical Prostatectomy Damages through Tissue-specific Paracrine Mechanisms,” Stem Cells 34(2):392-404; Zhang, J., Kang, N., Yu, X., Ma, Y. and Pang, X. 2017. “Radial Extracorporeal Shock Wave Therapy Enhances the Proliferation and Differentiation of Neural Stem Cells by Notch, PI3K/AKT [phosphatidylinositol 3 protein kinase B], Wnt/β-catenin [calcium.wingless, and Int-1—noncanonical pathway] Signaling,” Scientific Reports 7(1):15321; Zissler, A., Steinbacher, P., Zimmermann, R., Pittner, S., Stoiber, W., Bathke, A., and Sanger, A. M 2017. “Extracorporeal Shock_Wave Therapy Accelerates Regeneration after Bathke Skeletal Muscle Injury,” American Journal of Sports Medicine 45(3):676-684; Pajovic, B., Radojevic, N., Dimitrovski, A., and Vukovic, M. 2016, “Comparison of the Efficiency of Combined Extracorporeal Shock-wave Therapy and Triple Therapy Versus Triple Therapy Itself in Category III B Chronic Pelvic Pain Syndrome (CPPS),” Aging Male 19(3):202-207; Xin, Z.-C., Yong, D. X., Lin, G., Lue, T.-F., and Guo, Y. L. 2016. “Recruiting Endogenous Stem Cells: A Novel Therapeutic Approach for Erectile Dysfunction,” Asian Journal of Andrology 18(1):10-15; d'Agostino, M. C., Craig, K., Tibalt, E., and Respizzi, S. 2015. “Shock Wave as Biological Therapeutic Tool: From Mechanical Stimulation to Recovery and Healing, through Mechanotransduction,” International Journal of Surgery 24 (Part B):147-153; Song, M., Lim, J., Yu, H. Y., Park, J., Chun, J. Y., and 12 others 2015. “Mesenchymal Stem Cell Therapy Alleviates Interstitial Cystitis by Activating Wnt Signaling Pathway,” Stem Cells and Development 24(14): 1648-1657; Takayanagi. A., Sasaki. M., Kataoka-Sasaki. Y., Kobayashi, K., Matsuda, Y., and 4 others 2015. “Intravenous Preload of Mesenchymal Stem Cells Rescues Erectile Function in a Rat Model of Cavernous Nerve Injury,” Journal of Sexual Medicine 12(8):1713-1721; Kim, J. H., Lee, H. J., and Song, Y. S. 2014. “Treatment of Bladder Dysfunction Using Stem Cell or Tissue Engineering Technique,” Korean Journal of Urology 55(4): 228-238; Schuh, C. M., Heher, P., Weihs, A. M., Banarjee, A., Fuchs, Cl, and 6 others 2014. “In Vitro Extracorporeal Shock Wave Treatment Enhances Stemness and Preserves Multipotency of Rat and Human Adipose-derived Stem Cells,” Cytotherapy 16(12):1666-1678; Song, M., Heo, J., Chun, J. Y., Bae, H, S., Kang, J. W., and 5 others 2014. “The Paracrine Effects of Mesenchymal Stem Cells Stimulate the Regeneration Capacity of Endogenous Stem Cells in the Repair of a Bladderoutlet-obstruction-induced Overactive Bladder,” Stem Cells and Development 23(6):654-663; Speed C. 2014. “A Systematic Review of Shockwave Therapies in Soft Tissue Conditions: Focusing on the Evidence,” British Journal of Sports Medicine 48(21):1538-1542; Weihs, A. M., Fuchs, C., Teuschl, A. H., Hartinger, J., Slezak, P., and 5 others 2014. “Shock Wave Treatment Enhances Cell Proliferation and Improves Wound Healing by ATP Release-coupled Extracellular Signal-regulated Kinase (ERK) Activation,” Journal of Biological Chemistry 289(39):27090-27104; Birder, L. and Andersson, K. E. 2013. “Urothelial Signaling,” Physiological Reviews 93(2):653-680; Tepeköylü, C., Wang, F. S., Kozaryn, R., Albrecht-Schgoer, K., Theurl, M., and 7 others 2013. “Shock Wave Treatment Induces Angiogenesis and Mobilizes Endogenous CD31/CD34-positive Endothelial Cells in a Hindlimb Ischemia Model: Implications for Angiogenesis and Vasculogenesis,” Journal of Thoracic and Cardiovascular Surgery 146(4):971-978; Yoshida, S., Aihara, K., Ikeda, Y., Sumitomo-Ueda, Y., Uemoto, R., and 14 others 2013. “Androgen Receptor Promotes Sex-independent Angiogenesis in Response to Ischemia and is Required for Activation of Vascular Endothelial Growth Factor Receptor Signaling,” Circulation 128(1):60-71; Hayashi, D., Kawakami, K., Ito, K., Ishii, K., Tanno, H., and 7 others 2012. “Low-energy Extracorporeal Shock Wave Therapy Enhances Skin Wound Healing in Diabetic Mice: A Critical Role of Endothelial Nitric Oxide Synthase” Wound Repair and Regeneration 20(6):887-895; Qiu, X., Fandel, T. M., Ferretti, L., Albersen, M., Orabi, H., and 5 others 2012. “Both Immediate and Delayed Intracavernous Injection of Autologous Adipose-derived Stromal Vascular Fraction Enhances Recovery of Erectile Function in a Rat Model of Cavernous Nerve Injury,” European Urology 62(4):720-727; Marszalek, M., Berger, I., and Madersbacher, S. 2009, “Pain Syndrome: Finally, the Magic Bullet?,” European Urology 56(3):425-426; Zimmermann, R., Cumpanas, A., Miclea, F., and Janetschek, G. 2009. “Extracorporeal Shock Wave Therapy for the Treatment of Chronic Pelvic Pain Syndrome in Males: A Randomised, Double-blind, Placebo-controlled Study,” European Urology 56(3):418-424; Komori, K., Tsujimura, A., Takao, T., Matsuoka, Y., Miyagawa, Y., and 3 others 2008. “Nitric Oxide Synthesis Leads to Vascular Endothelial Growth Factor Synthesis via the NO/Cyclic Guanosine 36′,5′-monophosphate (cGMP) Pathway in Human Corpus Cavernosal Smooth Muscle Cells,” Journal of Sexual Medicine 5(7):1623-1635; Aicher, A., Heeschen, C., Sasaki, K., Urbich, C., Zeiher, A. M., and Dimmeler, S. 2006. “Low-energy Shock Wave for Enhancing Recruitment of Endothelial Progenitor Cells: A New Modality to Increase Efficacy of Cell Therapy in Chronic Hind Limb Ischemia,” Circulation 114(25):2823-2830; Wang, C.-J., Wang, F.-S., Yang, K. D., Weng, L.-H., Hsu, C.-C., Huang, C.-S., and Yang, L.-C. 2003. “Shock Wave Therapy Induces Neovascularization at the Tendon-Bone Junction. A Study in Rabbits,” Journal of Orthopaedic Research (New York, N. Y.) 21(6):984-989).

Low intensity shock wave therapy, ordinarily applied as lithotripsy to break up urinary stones, has been found, sometimes combined with the administration of stem cells, angiogenic drugs, platelet-rich plasma, and/or oxygenating agents, all of which the automatic disorder response system can administer in a tightly controlled manner, to ameliorate other problems associated with a chronically hypoxic bladder (see, for example, Li, G, and Man, L. 2021. “Low-intensity Extracorporeal Shock Wave Therapy for Male Chronic Pelvic Pain Syndrome: A Systematic Review and Meta-analysis,” Translational Andrology and Urology 10(3):1202-1211. Lu, J.-H., Chueh, K.-S., Chuang, S.-M., Wu, Y.-H., Lin, K.-L. and 5 others 2021, Op cit.; Guu, S.-J., Liu, C.-C., Juan, Y.-S., Li, C.-C., and Tsai, C.-C. 2020. “The 12-month Follow-up of the Low-intensity Extracorporeal Shockwave Therapy in the Treatment of Patients with Chronic Pelvic Pain Syndrome Refractory to 3-As [antibiotics, alpha-blockers, and anti-inflammatories] Medications,” Aging Male 23(5):793-800; Homma, Y., Akiyama, Y., Tomoe, H., Furuta, A., Ueda, T., and 7 others 2020. “Clinical Guidelines for Interstitial Cystitis/Bladder Pain Syndrome,” International Journal of Urology 27(7)-578-589, online at https://doi.org/10.1111/iju.14234; Ke, Q.-S., Jhang, J.-F., Lin, T.-Y., Ho, H.-C., Jiang, Y.-H., Hsu, Y.-H., and Kuo, H.-C., 2019. “Therapeutic Potential of Intravesical Injections of Platelet-rich Plasma in the Treatment of Lower Urinary Tract Disorders Due to Regenerative Deficiency,” Ci Ji Yi Xue Za Zhi [Buddhist Compassion Relief Tzu Chi Foundation Medical Journal (Taiwan)] 31(3):135-143; Lin, C. C., Huang, Y. C., Lee, W. C., and Chuang, Y. C. 2020. “New Frontiers or [sic] in the Treatment of Interstitial Cystitis/Bladder Pain Syndrome—Focused on Stem Cells, Platelet-rich Plasma, and Low-Energy Shock Waves,” International Neurourology Journal 24(3):211-221; Jhang, J.-F., Lin, T.-Y., and Kuo, H.-C. 2019. “Intravesical Injections of Platelet-rich Plasma is Effective and Safe in Treatment of Interstitial Cystitis Refractory to Conventional Treatment—A Prospective Clinical Trial,” Neurourology and Urodynamics 38(2):703-709; Jhang, J. F., Wu, S. Y., Lin, T. Y., and Kuo, H. C. 2019. “Repeated Intravesical Injections of Platelet-rich Plasma are Effective in the Treatment of Interstitial Cystitis: A Case Control Pilot Study,” Lower Urinary Tract Symptoms 11(2):042-047; Liu, T., Shindel, A. W., Lin, G., and Lue, T. F. 2019. “Cellular Signaling Pathways Modulated by Low-intensity Extracorporeal Shock Wave Therapy,” International Journal of Impotency Research 31(3):170-176; Long, C.-Y., Lin, K.-L., Lee Y.-C., Chuang S.-M., Lu J.-H., and 5 others 2020. “Therapeutic Effects of Low Intensity Extracorporeal Low Energy Shock Wave Therapy (LiESWT) on Stress Urinary Incontinence,” Scientific Reports 10(1):5818; Yuan, P., Ma, D., Zhang, Y., Gao, X., Liu Z., and 5 others 2019. “Efficacy of Low-intensity Extracorporeal Shock Wave Therapy for the Treatment of Chronic Prostatitis/Chronic Pelvic Pain Syndrome: A Systematic Review and Meta-analysis,” Neurourology and Urodynamics 38(6):1457-1466; Wang, B., Zhou, J., Banie, L., Reed-Maldonado, A. B., Ning, H., and 9 others 2018. “Low-intensity Extracorporeal Shock Wave Therapy Promotes Myogenesis through PERK/ATF4 [mammalian protein kinase PKR-like ER [endoplasmic reticulum] kinase/activating factor 4 (tax-responsive enhancer element B67)] Pathway,” Neurourology and Urodynamics 37(2):699-707; Chung, E. and Wang, J. 2017. “A State-of-art Review of Low Intensity Extracorporeal Shock Wave Therapy and Lithotripter Machines for the Treatment of Erectile Dysfunction,” Expert Reviews of Medical Devices 14(12):929-934; Jin, Y., Xu, L., Zhao, Y., Wang, M., Jin, X., and Zhang, H. 2017. “Endogenous Stem Cells were Recruited by Defocused Low-energy Shock Wave in Treating Diabetic Bladder Dysfunction,” Stem Cell Reviews and Reports 13(2):287-298; Wang, B., Ning, H., Reed-Maldonado, A. B., and 6 others 2017. “Low-intensity Extracorporeal Shock Wave Therapy Enhances Brain-derived Neurotrophic Factor Expression through PERK/ATF4 Signaling Pathway,” International Journal of Molecular Sciences 18(2):433; Wang, H. J. Lee, W. C., Tyagi, P., Huang, C. C., and Chuang, Y. C. 2017. “Effects of Low Energy Shock Wave Therapy on Inflammatory Moleculars, Bladder Pain, and Bladder Function in a Rat Cystitis Model,” Neurourology and Urodynamics 36(6):1440-1447; Donmez, M. I., Inci, K., Zeybek, N. D., Serkan Do\check{g}an, H., and Ergen, A. 2016. The Early Histological Effects of Intravesical Instillation of Platelet-rich Plasma in Cystitis Models,” International Neurourology Journal 20(3):188-196; Yahata K, Kanno H, Ozawa H, Yamaya, S., Tateda, S., and 3 others 2016. “Low-energy Extracorporeal Shock Wave Therapy for Promotion of Vascular Endothelial Growth Factor Expression and Angiogenesis and Improvement of Locomotor and Sensory Functions after Spinal Cord Injury,” Journal of Neurosurgery. Spine 25(6):745-755; Chen, Y. T., Yang, C. C., Sun, C. K., Chiang, H. J., Chen, Y. L., and 7 others 2014. “Extracorporeal Shock Wave Therapy Ameliorates Cyclophosphamide-induced Rat Acute Interstitial Cystitis though Inhibiting Inflammation and Oxidative Stress in in-Vitro and in in-Vivo Experiment Studies” American Journal of Translational Research 6(6):631-648; Cruz, F. and Nitti, V. 2014. “[Chapter 5] Clinical Data in Neurogenic Detrusor Overactivity (NDO) and Overactive Bladder (OAB),” Neurourology and Urodynamics 33 Supplement 3:S26-S31)

Another constraint in the nontargeted, or systemically dispersed administration of a drug is that the dose cannot be less than would affect the target tissue to a therapeutically meaningful extent but at the same time must not be so great as to adversely affect nontargeted tissue. Compelling underdosing to treat intended tissue while encouraging overdosing adverse to unintended tissue poses risks from both standpoints. In fundamental contrast to this limitation, directly pipe-targeting a drug to the target allows the dose, to which—absent a number of methods for eliminating it entirely—nontargeted tissue might be exposed to no more than a trace amount, to be freely set to that level optimal for the condition of the tissue when targeted.

Delivery thus means that drug selection and dosing need never make concessions to drug-drug or drug-nutrient interactions or the effect on nontargeted tissue. Unfortunately, in the treatment of cancer, the impetus to use the fewest drugs in the lowest dose must often be disregarded to save the patient (see, for example, Hahn, M. and Glatstein, E. 2005. “Principles of Radiation Therapy,” in Harrison's Principles of Internal Medicine, New York, N.Y.: McGraw-Hill, 16th Edition, pages 482-489). Another effect on pharmacy is the impetus to develop liquid drugs at high concentrations for automatically controlled microdrop drip release to allow minimization in the size of implanted drug reservoirs and thus avert the need for the patient to wear a power and pump pack.

A key factor in treatment with the aid of such means is that drug and electrical discharge delivery is targeted, that is, conveyed directly to the treatment site or sites, eliminating electrical or drug takeup and reaction by nontargeted tissues. The view reciprocal to that specified above is no less important. By allowing dosing that substantially omits nontargeted tissue, targeting considerably expands the utility of existing drugs, often in smaller doses, with fewer side effects, and at less expense. Moreover, the avoidance of a dependency upon intrinsic affinity combined with adverse side effects to nonaffiant tissue should not only optimize the efficacy of drugs long in use but expedite the approval of new drugs, significantly reducing the expense of pharmaceutical development.

Ductus side-entry connectors allow the secure connection of synthetic tubing to anatomical tubular structures, to include blood vessels and digestive conduits. An automatic ambulatory prosthetic disorder response system makes possible the continuous automatic monitoring, recording, and application of therapy that previously could be applied only while the patient remained confined to the clinic. The utility of state of the art ambulatory diagnostic tools, such as Holter monitors and event recorders (see, for example, The Merck Manual 18th edition, 2006, section 7, chapter 70, “Cardiovascular Tests and Procedures, page 597) in capturing diagnostic information without the need for continued vigilance in a hospital setting is thus generalized to the diagnosis of each in number of morbidities as well as the totality thereof.

Moreover, the therapy itself is availed of the fundamental benefits of direct pipeline delivery to the target, to include the ability to optimize dosages without exposure to nontargeted, often vulnerable tissue. The avoidance of adverse side effects, drug-drug, and drug-nutrient interactions caused by drugs physically isolated from the body other than the end organ or gland of the blood supply into which the pipeline empties is a fundamentally liberating consequence for drug development, application, and prescription. These interactions severely constrain prescription to the point of hesitancy so that that the fewest drugs in the lowest are recommended, and in so doing, may result in the withholding of a large enough dose, an additional drug or drugs, or the use of these additional drugs in greater concentration that would more contribute to patient relief.

To allow the use of an essential drug may require reviewing the combination of drugs prescribed to determine on a drug-by-drug basis whether any of the others might be eliminated or substituted. Moreover, when evidence of an adverse interaction appears, serum concentration testing may become necessary to identify the source of the problem (see, for example, The Merck Manual 18th edition, 2006, pages 2516, 2517). In affording strict control over which drugs and nutrients may be allowed to come into contact, direct pipeline targeting eliminates all potential for the emergence of such hindrances. Some side effects must be reported by the patient; the ability to detect side effects is limited by the applicability of sensors—side effects such as confusion, changes in vision, and dizziness make automated detection difficult if not impossible.

Even in the clinic, to implement such a system requires stable, durable, and leak-free junctions between the synthetic materials of the end-connectors and tissue. Ductus side-entry jackets and nonjacketing side-entry connectors must be long-lived, sufficiently stable in dimensions, reasonably accommodate or conform to rather than encroach upon neighboring tissue, and minimally excite foreign body tissue reactions or otherwise cause discomfort. The objective in this regard is that the freely moving and fully ambulatory patient is best oblivious to the system.

That the limitation of drug dosing to less than optimal levels for no inherent reason, but rather because these cannot be isolated from other tissues during delivery to the target for fear of side effects has been overcome should result in new levels of efficacy for many drugs that have long been known but never allowed to be delivered in the dose required to be most effective. If the disease is systemic and the original site of disease from which it can spread or metastasize is directly targeted, then a background dose of the drug or drugs is circulated. Similarly, the potential for metastasis of a carcinoma or sarcoma is suppressed with a systemic dose much reduced compared to that conventional, and possibly a regional dose somewhat higher in concentration, while the lesion itself is directly assailed with as concentrated an anticancer drug or drugs as will not provoke injury equally worrisome as the cancer itself.

The isolated delivery and targeting of relatively high dose rate antineoplastics or anticarcinogens is through radiation shielded passages and connectors, described and illustrated in copending application Ser. No. 15/998,002 entitled Ductus Side-entry Jackets and Prosthetic Disorder Response Systems, and Ser. No. 14/998,495, entitled Nonjacketing Side-entry Connectors and Prosthetic Disorder Response Systems, both of which show radiation shielding devised to break down over time as well shielding that will not. Numerous issues omitted in this application, to include the various implementations of radiation and the use of superparamagnetic nanoparticle-carried drugs make it essential to review these applications.

For biomedical engineers and technically oriented internists, among others, the prerequisites to make possible the realization of a fully or almost fully implanted automated system to suppress a single chronic disease using negative feedback control would appear not simple but still clear enough to be dealt with on the basis of experience and intuition. Mostly what is needed is some technical background about automation, a good understanding of the problem and its interactions from the standpoint of internal medicine, an awareness of the availability of practical connectors, drivers, and analyte sensors, or detectors, that would be needed to institute the system so that it would continue to function over an indefinite but very long period.

However, even to create this relatively simple nonspecific monomorbid system, all of the essential components, described and illustrated in detail in the copending applications identified above, will usually not have been approved for implantation in humans and therefore remain unavailable, while references to various sensors which are available are cited in the literature. Here is taken up comorbid disease in which all of the components interact, making the maintaining of a running overall summary diagnosis to allow optimal treatment from the lowest level to that comprehensive not just complex but demanding of time not ordinarily granted to a single patient.

Here the comorbities represent the upward arms or channels of progressively more highly integrated and coordinated data. A fully implanted hierarchical control system of the kind indicated comprises three major components:

1. A fully implanted set of sensors to generate ground level diagnostic data specific to each component morbidity. Two morbidities may require more than just one or two sensors each, and an increase in the number of sensor can determine the number of nodes, or subcontrollers, at higher levels into which they feed.

2. The lower level nodes coordinate the ground level diagnostic sensor output data sent to them from the ground level. The larger the number of sensors, the greater is the probability that more than one node at the next or two next higher levels will intervene between the ground level and the master controller. Moving up the hierarchy, the degree of data integration becomes greater and the number of nodes fewer. First, the data for each morbidity is integrated, then the data for the other morbidity or morbidities must be coordinated with this data. At the penultimate level in the hierarchy, the data from the component morbidities is integrated and passed up to the master controller for remedial action.

Based upon the prescription-program, the master controller, which can identify problems at any subordinate level, evaluates the summary data covering the combination of morbidities, compares the values across the morbidities to the those in the normal range, and passes the adjustments to be effected down through the levels as ‘motor’ commands to effect the adjustment. More specifically, the master controller translates its comprehensive data into the commands issued to the system drug reservoir outlet motors—or if delivered by gravity feed, opens the stopcock of the reservoir, or energizes and monitors the electrical end-effectors assigned—and monitors each. When the preferred end-values cannot be specified, the master node, or master controller, can be programmed to apply a sequence of test doses at different levels to find that which yields the best overall result.

3 The third major component is the hardware, the pipeline distribution system consisting of a set of catheteric blood and drug pipelines to deliver the drugs and/or other agents injected at a body surface port such as those shown in copending application Ser. No. 15/998,002, entitled Ductus Side-entry Jackets, FIGS. 27 and 28 therein and copending application Ser. No. 16/873,914 entitled Vascular Valves and Servovalves—and Prosthetic Disorder Response Systems, FIGS. 26A thru 26C therein into the drug reservoirs to pass through the ductus fluid lines leading to the side-entry and nonjacketing connectors and electrical end-effectors described in the copending applications cited and through these into the target nidi, tissues, or their blood supply. The entry holes respective of each pipeline can also serve to allow an endoscope to be passed down to the treatment site.

Through this distribution system, the control system effectuates drug and nondrug, such as electrostimulatory or neuromodulatory therapy that is directly targeted by transmission through a fluid pipe or electrical line. Extraordinarily, commands can be transmitted by radio, generally ‘Bluetooth’. Total body networks, transcutaneous, or transdermal, energy transfer, remote diagnostics, and medical telemetry are covered.

If it poses a potential problem, any trace residue, even radioactive, not taken up within the parenchyma, endothelium, or urothelium and as a result continues to pass into the venous drainage is easily eliminated through a number of methods described in the foregoing applications. The incorporation of service or accessory channels allows all pipelines and end-connectors to be intermittently drip-fed or occasionally flushed through with clot, crystal, and/or biofilm buildup solvent as well as allow the intermittent addition to the primary drug of an adjuvant, for example. In this regard, service or accessory channels eliminate a major drawback in the usability of polymeric tubing to serve as small shunts and bypasses.

The first of these was described in detail in the copending applications cited, which not only clarify the structure of the different type end connectors used but the applications of these over a broad spectrum of internal medicine. he delivery system must incorporate means for connection to native tissue that should never require followup maintenance that cannot be accomplished with the service or accessory channels provided. In the copending applications specified, system jacketing and nonjacketing connectors, the different types and the numerous applications of each type have been described and illustrated in detail.

In the treatment of monomorbid disease, such as diabetes or heart failure, the system master controller is a microcontroller. Where both of these conditions, elevated blood pressure, and atherosclerosis combine in metabolic disease—in light of its prevalence, an ideal example—a more capable controller, a microprocessor, programmed at a lower level to address each disease component and at a higher level to cross-level treatment among the component morbidities to achieve the best overall homeostatic condition is used. The implication that the system maintains a running record of drug release/outcome relations which can be printed out as essential for diagnostics and the formulation of updated or new prescription-programs is correct.

By now, the concept of hierarchical control is over a half century old, and appears to have been limited in application to the programming of autonomous industrial robots and ‘rovers’ for deployment on the moon and other planets (Mesarovic, M. D., Marco, D., and Tashahara, Y. 1970. Theory of Hierarchical Multilevel Systems, Academic Press: New York, N.Y.), and the resolution of performance optimization issues in production plants. Similarly to the industrial applications of hierarchical control where the object was to progressively adjust an unfolding or building process as would best approximate the specified end result, in medicine, the result is not designed but given by nature as a condition of normalcy. There appears to be no evidence that the concept of hierarchical control was ever contemplated as a powerful tool to coordinate the remedial action to be taken on the basis of often complex data which is the ordinary situation in internal medicine.

Even though hierarchical control has been around for decades, without means for safely, durably, and connecting to native ductus along the vascular tree through a secure and leak-free junction with catheteric drug and blood pipelines rendered invulnerable to the accumulation of clot, crystal, and/or biofilm, the relation, indeed realization of the possibility to apply hierarchical networked feedback to automatic drug delivery has remained unfeasible. Sensors can be collocated with the means that make possible the targeted release of drugs to the location respective of each.

For this reason, immediate and automatic remedial drug delivery, not just information as to the status of the patient, can be achieved. As will become clear, the strategically located sensors, ductus side-entry connection pump-pairs and jacket sets to be described make possible the targeted delivery of drugs through automatic response that is immediate. Were the condition to exceed the range of adjustment for which the system had been set, the exigent readings can be transmitted to a clinician able to adjust the dosing by remote control.

Every tissue in the body is either part of and therefore directly, or supplied by and therefore indirectly, accessible through vessels and/or ducts. There is no disease in which vascular and other supply and drainage lines are uninvolved and signal the local dysfunction to higher control centers. Symptoms even appear in bodily systems that would seem qualitatively unlike and remote from that of origin. Regional enteritides can induce arthritis. Osteoporosis and Paget's disease of bone (osteitis deformans), for example, are disorders often secondary to endocrine disease that affect the skeleton.

If arterial applications are stressed, it is because of the disproportional involvement of vessels in death from disease. No bodily conduit, to include the smallest, is analogous to inert plumbing; all are integrated into a hierarchy of negative feedback loops from the brain down to the individual cells to actively interact with the passing contents (see, for example, Jameson, J. L. 2005. “Principles of Endocrinology,” in Harrison's Principles of Internal Medicine, New York, N.Y.: McGraw-Hill, page 2072).

Every bodily conduit communicates directly or indirectly with all the tissues in the body—not just by transmitting luminal contents, but by signaling local function to higher control centers. In endothelial function, for example, the linings of blood and lymphatic vessels actively secrete vasodilators such as relaxing factor (like indistinguishable from nitric oxide), bradykinin, potassium ions, and adenosine and vasopressors or vasoconstrictors such as endothelins, epinephrine, norepinephrine, dopamine, thromboxane, and insulin, all tied into coordinated feedback loops, which continuously adjust the degree of contraction, hence, the blood pressure.

That vessel wall, segment, and organ drug targeting has not progressed beyond the drug eluting stent is due to an inadequacy of methods and means for limiting drug delivery to the site that requires treatment and would allow different drugs to be delivered in doses not limited by intolerances to tissues beyond the target area. Whether access through ‘keyhole’ incisions at the body surface is more invasive than transluminal access may not be true.

In addition to communication affected by the autonomic nervous system, the luminal wall can release signaling proteins, such as chemokines and interleukins, and the luminal contents can include enzymes, hormones, cells containing cytokine signaling proteins, and so on, so that remote tissues are affected as well. As a result, there is no disease in which bodily conduits are uninvolved. No bodily conduit is analogous to inert plumbing; all are integrated into a hierarchy of negative feedback loops from the individual cells to the brain to actively and appropriately interact with the constitution, pressure, and velocity of passing contents.

Collaterally, vasopressin, or antidiuretic hormone, produced in the hypothalamus and released by the pituitary gland in response to a decrease in blood volume exerts a pressor effect and acts as a diuretic by reducing the volume of urine, thereby conserving the volume of blood. That the caliber of blood vessels, for example, is adaptive locally as well as systemically demonstrates that control is effected by a hierarchy of control loops wherein those subordinate interact with those progressively more encompassing until the center just above the brainstem is reached. Central mechanisms initiate the release of circulating vasoconstrictors or vasodilators that cause the linings of blood vessels to contract or relax in response to the condition of the circulatory system, which includes cardiac output, partial pressures of oxygen and carbon dioxide, and the existing concentration of hormones and electrolytes in the blood.

Blood pressure as the product of cardiac output and peripheral resistance subsumes numerous interrelated contributory closed loop actions responsive to emotional state, level of exertion, temperature, metabolism as affected by ingesta, disease, medication, and gas exchange in the lungs effected by cellular level feedback between every cell and its immediate environment. Maintaining normal function in the walls of bodily conduits is thus central to and inseparable from maintaining normal function. Much the same hierarchical integration mutates between the wider physiological context and any other bodily conduit, whether ureter, gamete transporting duct, the airway, choroid plexus and arachnoid villi, or lymphatic vessel.

While the body is able to compensate for numerous forms of degradation, such as those associated with aging, a failure to produce an essential enzyme or to produce an essential protein as the result of a genetic defect or progressively emerging alteration, for example, is sufficiently anomalous that the body lacks sufficient responsive measures. Thus, up to the degree of deviation that can be accommodated, an atherosclerosed artery is continuously remodeled, preserving its luminal diameter, for example, but the inadequate synthesis of insulin, resulting in diabetes, or tyrosine, resulting in phenylketonuria, for example, demand human intervention.

Such anomalous defects, to which the body has limited if any adaptive or accommodative compensatory response, account for much of internal medical practice. Application to controlled steering of a prosthetic hand has been addressed (Light, C. M., Chappell, P. H., Hudgins, B., and Engelhart, K. 2002. “Intelligent Multifunction Myoelectric Control of Hand Prostheses,” Journal of Medical Engineering and Technology 26(4):139-146; Chappell, P. H. and Kyberd, P. J. 1991. “Prehensile Control of a Hand Prosthesis by a Microcontroller,” Journal of Biomedical Engineering 13(5):363-369), but nowhere does there appear the application of hierarchical control to continuous adjustment in the execution of a prescription or any end motion-unrelated medical use.

A system for the delivery of drugs under the control of a hierarchical control system is analogous to the kind of system used to control a remote vehicle, for example. Such a system, where data is collected as to the best overall outcome across a plurality of morbidities treated with various combinations of drugs administered is analogous to the kind used to control a remote vehicle over uneven terrain, where data concerning the contour of the ground covered and the orientative response thereto is continuously collected for use to adapt for and optimize continued level transit, for example.

While not as applied to internal medicine, hierarchical control, whereby sensors send input data up through levels of more encompassing data integration and commands usually proceed in reverse down through the same chain, is a well developed field (see, for example, Raisch, J., Schmuck, A.-K., Gromov, D., and Geist, S. 2021. “Hierarchical Control Theory,” online at https://www.mpi-magdeburg.mpg.de/95036/Hierarchical-Control-Theory; Dellaert, F. 2020. “Hierarchical Control,” online at https://www.cc.gatech.edu/˜dellaert/07F-Robotics/Schedule_files/02-Hierarchical Control.ppt.pdf; Merel, J., Botvinick, M. and Wayne, G. 2019. “Hierarchical Motor Control in Mammals and Machines,” Nature Communications 10:5489, online at https://doi.org/10.1038/s41467-019-13239-6; Wayne, G. and Abbott, L. F. 2014. “Hierarchical Control Using Networks Trained with Higher-level Forward Models,” Neural Computation 26(10):2163-2193; Erez, T. 2011. “Optimal Control for Autonomous Motor Behavior,” Doctoral Dissertation, School of Engineering and Applied Science, Washington University, St. Louis, Mo.; Hou, Z.-G., Gupta, M. M. Nikiforuk, P. N., and Tan, M. H. 2007. “A Recurrent Neural Network for Hierarchical Control of Interconnected Systems,” Institute of Electrical and Electronics Engineers Transactions on Neural Networks 18(2):466-481; Schlenoff, C., Albus, J., Messina, E., Barbera, A. J., Madhavan, R., and Balakirsky, S. 2006. “Using 4D/RCS to Address AI Knowledge Integration,” Artificial Intelligence Magazine 27(2):71-81; Aguilar, J., Cerrada, M., Mousalli, G., Rivas, F., and Hidrobo, F. 2005. “A Multiagent Model for Intelligent Distributed Control Systems,” 191-197; http://www.mpi-magdeburg.mpg. de/95036/Hierarchical-Control-Theory; Meystel, A. M. and Albus, J. S. 2002. Intelligent Systems, New York, N.Y.: John Wiley and Sons; Albus, J. S. 2000. “4-D/RCS [Four Dimensional Real-time Control System] Reference Model Architecture for Unmanned Ground Vehicles,” in Proceedings of the 2000 IEEE International Conference on. Robotics and Automation, New York, N.Y.: Institute of Electrical and Electronics Engineers; volume 4, datalogue number 00CH37065, pages 3260-3265; Takahashi, Y. and Asada, M. 1999. “Behavior Acquisition by Multi-layered Reinforcement Learning,” in Proceedings of the 1999 IEEE International Conference on Systems, Man, and Cybernetics, New York, N.Y.: Institute of Electrical and Electronics Engineers; pages 716-721; Albus, J. S. 1996. “The Engineering of Mind”. From Animals to Animats 4,” in (Maes, P., Mataric, M. J., Meyer, J.-A., Pollack, J., and Wilson, S. W. (eds.) Proceedings of the Fourth International Conference on Simulation of Adaptive Behavior (Complex Adaptive Systems): Cambridge, Mass.: MIT Press; Albus, J. S. and Meystel, A. M. 1996. “A Reference Model Architecture for Design and Implementation of Intelligent Control in Large and Complex Systems,” International Journal of Intelligent Control and Systems 1(1):15-30; Albus, J. S. 1995. “RCS: A Reference Model Architecture for Intelligent Systems, Association for the Advancement of Artificial Intelligence Technical Report SS-95-02, available at http://aaaipress.org/Papers/Symposia/Spring/1995/SS-95-02/SS95-02-001.pdf; Albus, J. S. 1993. “A Reference Model Architecture for Intelligent Systems Design,” Chapter 2, pages 27-56 in Antsaklis, P. J. and Passino, K. M., eds., An Introduction to Intelligent and Autonomous Control, Baltimore, Md.: Wolters Kluwer Academic Publishers; Hayes-roth, F., Erman, L., and Terry, A. 1992. “Distributed Intelligent Control and Management (DICAM) Applications and Support for Semi-automated Development,” in Keller, R/M. (ed.), Working Notes from the 1992 AAAI [Association for the Advancement of Artificial Intelligence] Workshop on Automating Software Design, National Aeronautics and Space Administration Technical Reports Server Document ID 19930008310; Baxt, W. G. 1991. “Use of an Artificial Neural Network for the Diagnosis of Myocardial Infarction, Annals of Internal Medicine 115(11):843-848; Maclin, P. S., Dempsey, J., Brooks, J., and Rand, J. 1991. “Using Neural Networks to Diagnose Cancer,” Journal of Medical Systems 15(1):11-19; Reggia, J. A. and Peng, Y. 1987. “Modeling Diagnostic Reasoning: A Summary of Parsimonious Covering Theory. Computer Methods and Programs in Biomedicine 25(2):125-134; Jones, A. T. and McLean, C. R. 1986. “A Proposed Hierarchical Control Model for Automated Manufacturing Systems,” Journal of Manufacturing Systems 5 (1): 15-25; Findeisen, W. 1984. “The Essentials of Hierarchical Control,” in Thoft-Christensen, P. (ed.), System Modelling and Optimization. Lecture Notes in Control and Information Sciences 59:38-61; Findeisen, W.; Bailey, F. N., Brdys, M., Malinowski, K., Tatjewoki, P., and Wozniak, A. 1980. Control and Coordination in Hierarchical Systems, Chichester, England/New York, N.Y.: John Wiley and Sons, Issue 9 of the International Series on Applied Systems Analysis, Wiley-Interscience; Miller, R. A. 1994. “Medical Diagnostic Decision Support Systems—Past, Present, and Future,” Journal of the American Medical Informatics Association 1(1):8-27; additional references provided below).

Control therefore is preferably of sensor response adaptive closed loop control over the delivery of each drug in the turret, control of the pump and turret stepper motors under open loop controlled. While the same degree of complexity and expense is not warranted in less serious cases, in a patient with an unstable life-threatening condition, adaptive response justifies the implantation of sensors tied into closed loops in a wireless body area network with automatic adaptive response in the dosing of each drug by means of a hard real time adaptive hierarchical, or nested, complex control system.

Provided with proper sensors properly located, such a system can be programmed to assimilate or ‘learn’ events as these are experienced, such as the action of a drug at an interval other than expected (Albus, J., Bostelman, R., Hong, T., Chang, T., Shackleford, W., and Shneier, M. 2006. “The LAGR [Learning Applied to Ground Robots] Project: Integrating Learning into the 4D/RCS [4 Dimensional Remote Control System] Control Hierarchy,” International Conference in Control, Automation and Robotics—ICINCO 06, Setubal, Portugal, available at http://www.nist.gov/customcf/get_pdf.cfm?pub_id=822702). Unless interrupted by an adverse event, the drug regimen continues unaffected. In such a hierarchical control system, the processors of a monolithic integrated circuit or microchip multicore microcontroller are partitioned to support one control node each, that programmed to function at the highest level of control as the master node sent the inputs from and having a time horizon comprehensive of the subordinate nodes, of which each contributes inputs to the pumps and jackets of the set based upon the sensors that feed it.

Meaning of an Automatic Adaptive/Predictive Ambulatory Prosthetic Disorder Response System

System desiderata and capabilities are addressed in different contexts and therefore appropriately addressed in different sections below such as that entitled Local and Systemic Implications of Automatic Sensor-driven Targeted Drug Delivery. With a portable (wearable, ambulatory) prosthetic disorder response system, the clinician specifies the target ductus, the drugs to be delivered to each, the dose regimen, and any additional factors pertinent thereto. According to the present concept, a pharmacist-programmer enters this into a program whereby each drug is provided in response to the conditions sensed. To deliver drugs automatically and adjust the dosing, the prescription, or adaptive drug delivery program, responds to diagnostic sensor feedback under the control of a medically adapted hierarchical (nodal, nested-levels) ‘intelligent’ hard real-time ‘pathfinding’ control system.

Depending upon the intricacy and frequency of differential control required of either pump in the pump and jacket set, each node controls either one of the pumps or the modular plug-in pump-pair as a subsystem in the pump-pack, usually cinched about the waist. While sensors, fluid lines, and connectors must be implanted, the control circuitry, power source, and pumps need not. Generally, the latter are implanted only when the condition or conditions treated are expected to persist to the end of life. In the case of progressive disease, the sensor-driven automatic drug delivery system spontaneously adjusts the intervals and dose of drugs in accordance with the prescription-program,

Representation in the drawing figures of system componentry as housed in a body surface-worn, or paracorporeal, pump, power, and/or control pack pertain no less to system implantation where these parts are much miniaturized to allow full, or closed-skin, implantation. To the extent practical, where comorbid conditions must be treated, each such component disease is assigned to a respective node and modular plug-in pump-pair and jacket set, and the master microprocessor programmed to coordinate the delivery of drugs among the nodes.

System Control of Multidrug Delivery System

Control therefore is preferably of sensor response adaptive closed loop control over the delivery of each drug in the turret, control of the pump and turret stepper motors under open loop controlled. While the same degree of complexity and expense is not warranted in less serious cases, in a patient with an unstable life-threatening condition, adaptive response justifies the implantation of sensors tied into closed loops in a wireless body area network with automatic adaptive response in the dosing of each drug by means of a hard real time adaptive hierarchical or nested complex control system.

Such a system, where data is collected as to the best overall outcome across a plurality of morbidities treated with various combinations of drugs administered is analogous to the kind used to control a remote vehicle over uneven terrain, where data concerning the contour of the ground covered and the orientative response thereto is continuously collected for use to adapt for and optimize continued level transit, for example (reference provided above and additional references cited below).

That is, with the proper sensors, such a system can be programmed to assimilate or ‘learn’ events as these are experienced, such as the action of a drug at an interval other than expected (Albus, J., Bostelman, R., Hong, T., Chang, T., Shackleford, W., and Shneier, M. 2006. “The LAGR [Learning Applied to Ground Robots] Project: Integrating Learning into the 4D/RCS [4 Dimensional Remote Control System] Control Hierarchy,” International Conference in Control, Automation and Robotics—ICINCO 06, Setubal, Portugal, available at http://www.nist.gov/customcf/get_pdf.cfm?pub_id=822702).

Unless interrupted by an adverse event, the drug regimen continues unaffected. In such a hierarchical control system, the processors of a monolithic integrated circuit or microchip multicore microcontroller are partitioned to support one control node each, that programmed to function at the highest level of control as the master node sent the inputs from and having a time horizon comprehensive of the subordinate nodes, of which each contributes inputs to the pumps and jackets of the set based upon the sensors that feed it.

In more advanced use—appropriate for the treatment of more complex comorbid disease where a certain apportionment of drugs among the treatment sites achieves the best overall consequence—the dispensing of drugs is automated, necessitating the implantation of additional components. Each component morbidity is assigned a channel or arm of control in a hierarchical control system which takes biosensor inputs at the lowest level. The inputs are passed for coordination to a control node, an intermediate chip-microcontroller, thence to a next higher node which coordinates treatment with its counterpart or counterparts in the other control channel or channels, the number thereof and need for coordination among these dependent upon the number of distinguishable morbidities.

These nodes then pass their outputs to a master node microprocessor. By analogy with the nervous system, such can be characterized as the system sensory function. Overall coordination of drug delivery (or other therapy, such as the application of heat), analogous to motor function, is then governed by the master node, a microprocessor which coordinates the inputs from the nodes next lower in rank and passes control signals down the channel in a motor sense, causing implanted drug reservoir outlet valves or pumps to release the drugs as directed.

When the condition of the patient allows, fitting of the system is preceded by an initial test period similar to that used in placing an electrostimulatory neuromodulator except that the question is not whether to implant the device but rather what combination drugs would best be used. The implanted system is used to test different drugs, the best overall result with minimal drug interaction thereby made discernible. This determined, a pharmacist-programmer prepares a prescription-program for execution by the master node. Immediately lesion- or original site of disease targeted and kept from the general circulation, medication delivered thus is substantially more effective in smaller doses and spares nontargeted tissue.

While in use independently, the local control module, itself a chip microcontroller, and associated components nevertheless represent a single node of the overall prosthetic disorder response system governed by a central microprocessor as the master control node. The later addition of another system module, such as requires a pump-pack and fluid lines to deliver synthetic mucus and digestive enzymes, then requires the activation of another node.

If the implanted or local control module is so capable, it continues to support the digestive module previously implanted, and has the new node added. When the digestive function need not be coordinated with the added function, it is most expeditious to allow the existing implant to continue to function independently. Otherwise, it is equally expeditious to place the previously implanted local control module as a node under the control of an added microcontroller in the pump-pack, or if the local controller is so capable, assign to it overall control. Later access governs the positioning of components.

Automatic disorder response system design strives for freedom from the impediment of a belt-worn power, control, and/or drug reservoir and outlet motor pack, conceding to the use thereof only when necessary. Implementation is preferably in the form of a fully, or closed-skin, system. Nevertheless, for pictorial clarity in copending applications pertaining to ductus side-entry jackets and nonjacketing side-entry connectors, system components are depicted in mechanical form.

The need for a paracorporeal pack arises when the number of system components exceeds that implantable without obtrusion into neighboring tissue to cause discomfort. Currently, conditions involving fewer morbities can be accommodated by a fully implanted system. Telemetric data transmission, a total body network, and the replacement of hard wires with short distance radio transmission can be obtained in small shape factor forms. Once transcutaneous power transfer allows the use of small antennae, this too can be implanted.

The relegation of nonimplanted components to an externally worn, or paracorporeal, body pack can include the hierarchical master node microprocessor, subordinate level node microcontrollers along each morbidity channel sent to the microprocessor, the power source, and external drug reservoirs or storage canisters, and pumps. Clearly, in the treatment of disease with the potential to result in death if not treated, a definite preference favors a fully implanted system, various means for fitting components inside the body such as tissue extension addressed in the copending applications cited above as well as herein.

Ambulatory Adaptive Prosthetic Disorder Response System Control

FIG. 4 provides a schematic of the control hierarchy for a single pump-pair in support of four jackets in the pump-pair and jacket set, the control program, that is, the prescription-program, of the master node, a microprocessor, determined by the specific or comorbid conditions to be treated. Nodes subordinate to the master node are generally microcontrollers. FIGS. 4 and 5 provide a schematic of the pump-pack, jacket set, and control system.

Unlike FIG. 6, in FIG. 5, only the control train is represented, the distinction between intra and extracorporeal elements omitted. An extracorporeal pack affords considerably more space and can hold a larger volume of numerous drugs, other therapeutic agents, and equipment maintenance solutions. While shown as carried in a body pack, the control hierarchy is implantable with the impediment of a pack eliminated.

When implanted, the contents labelled body pack at the lower left in FIG. 6 are miniaturized; otherwise, FIG. 5 applies no less to a fully implanted as to a body pack carry system. Also when implanted, to preclude complications due to encroachment upon or strangulation of tissue by wires, data intercommunication from the sensors and subordinate nodes and control signals from the master node are preferably by wireless, or Bluetooth transmission. For pictorial clarity, where the electrical and fluid lines between nodes and jackets are actually separate and distinct, those between nodes and jackets are shown as consolidated until finally led to each jacket, and remote sensors and auxiliary drug supply reservoirs have been omitted.

Electrical connectors, more remote sensors, drug supply reservoirs and outlet pumps controlled by the master node have been omitted. If provided with the requisite switching and valving, the fluid and electrical lines shown as shared could support each jacket independently but not simultaneously, the utility thereof contingent upon the condition or conditions to be treated; simultaneous capability is accomplished by furnishing the components necessary. Hard-wired intracorporeal electrical connections that would pose the risk of strangulating a structure or tissue intervening along the delivery route are preferably implemented by means of short range radio transmission such as provided by ‘Bluetooth.’

Such lines of communication can serve to interconnect local implanted sensors, their co-level nodes, intermediate nodes, and the hierarchical control master node, a microcontroller in uncomplicated or monomorbid, a microprocessor in comorbid disease. In FIGS. 5 and 6, single lines are electrical, or if it is found difficult to route the electrical lines without the risk of strangulating intervening structures, then connected by wireless ‘Bluetooth’ transmission rendered selective by difference in carrier frequency.

If virtually simultaneous operation cannot be achieved with a single carrier transmitter switching among the jackets, the microprocessor is provided with more than one transmitter. The addition of a module is coordinated with the module or modules already inserted; however, because when targeted to specific tissue, most if not all drugs are kept separate, the regimen overall as administered by the master controller over the nodes usually need not effect significant adjustments among these to accommodate the addition or removal of a power, control, and pump pack if such is needed.

While almost all practical applications will have been entirely translated into fully implanted miniaturized electronic versions of the equivalent mechanical components depicted in the drawing figures, by extracorporealizing numerous components, a pack does offer the benefits in the elderly and very young of both sparing the internal weight of these components (turreted pumps, batteries, possibly nodes) as well as freeing the application of changes in componentry from the need to reenter, even endoscopically.

Here a more literal mechanical representation of components which would almost always be implanted in miniaturized electronic forms equivalent to those mechanical that might be relegated to a belt-worn pack are primarily for pictorial clarity. Unless a practical power, control, and pump pack is provided with batteries which can be recharged through a small socket or transcutaneous-type energy transfer, a separate openable compartment can be provided to house disposable batteries. To prevent tinkering by a curious child, for example, the power, control, and pump pack can be locked, the key or keys maintained in the clinic.

A prescription-program can be executed by a multicore microcontroller of which each core or cog is programmed as a time division multiplexed node in the control hierarchy. Where magnetically susceptible carriers with or without a carried extractate (extractant) will be so small in volume as not to require removal, high energy product permanent magnets, ordinarily made of neodymium iron boron, are preferred. The addition of a module is coordinated with the module or modules already inserted; however, because when targeted to specific tissue, most if not all drugs are kept separate, the regimen overall as administered by the master controller over the nodes usually need not effect significant adjustments among these to accommodate the addition or removal of a pump-pack.

Such a prescription-program can be executed by a multicore microcontroller of which each core or cog is programmed as a time division multiplexed node in the control hierarchy. Where magnetically susceptible carriers with or without a carried extractate (extractant) will be so small in volume as not to require removal, high energy product permanent magnets as shown in copending application Ser. No. 15/932,172, entitled Integrated System for the Infixion and Retrieval of Implants, FIGS. 5, 6, 13, and 14 therein, ordinarily made of neodymium iron boron, are preferred.

Where magnetically susceptible, hence, extractate debris or detritus is drawn, a permanent magnet jacket that detains the debris, if necessary for dissolution with sodium hypochlorite isolated from tissue has a side grating such as that shown in copending application Ser. No. 15/932,172, FIG. 16 therein which allows the debris to be extracted with the aid of a powerful extracorporeal electromagnet. Generally, the debris if at all toxic will be equally so in the tissue surrounding the ductus; however, when extracted, it can be dispersed so as to reduce the immediate burden or concentration to a tolerable level or passage through normal excretion.

If the extractate debris is more toxic or radioactive, then electromagnetic extraction-jackets such as shown in copending application Ser. No. 15/998,002, entitled Ductus Side-entry Jackets and Prosthetic Disorder Response Systems FIGS. 13 thru 15 therein remove the extractate. The consecutive jackets along the ductus in FIG. 15 therein, ordinarily a vessel, are connected by a flush-line from a supply to a separate waste reservoir in the pump-pack, washing the pole of each electromagnet 75 where the debris accumulates along the way.

Instead of ambulatory means for continuously, automatically, immediately and autonomously responding to the condition sensed, readings are transmitted to a specialist for review and the writing of a prescription. The delay in this process is a conspicuous deficiency. Usually, the overall sequence in which the drugs are delivered to each side-entry jacket is maintained whether the jackets are placed along a single ductus, or where interrelated and interdependent organ systems are affected, along ductus belonging to different organ systems. In more complex situations, nested levels of program control, or nodes, each supporting a jacket incorporating symptom and remedial substance delivery and level-measuring sensors, are used.

Control System Options

The nodes can consist of time division multiplexed cores of a multicore microcontroller (see, for example, Schoeberl, M., Brandner, F., Sparsø, J., and Kasapaki, E. 2012. “A Statically Scheduled Time-division-multiplexed Network-on-chip for Real-time Systems,” pages 152-160, Networks on Chip (NoCS), 2012 Sixth Institute of Electrical and Electronics Engineers/ACM International Symposium on, Lyngby, Denmark, available at http://www.jopdesign.com/doc/s4noc.pdf; Sparso, J. 2012. “Design of Networks-on-chip for Real-time Multi-processor Systems-on-chip,” in 12th International Conference on Application of Concurrency to System Design, Hamburg, Germany pages 1-5; Paukovits, C. and Kopetz, H. 2008. “Concepts of Switching in the Time-triggered Network-on-chip,” in Proceedings of the 14th Institute of Electrical and Electronics Engineers International Conference on Embedded and Real-Time Computing Systems and Applications (RTCSA '08), Kaohsiung City, Taiwan, Republic of China, pages 120-129; Schoeberl, M. 2007. “A Time-triggered Network-on-chip,” in International Conference on Field-Programmable Logic and its Applications (FPL 2007), pages 377-382; Kopetz, H. and Bauer, G. 2003. “The Time-triggered Architecture,” Proceedings of the Institute of Electrical and Electronics Engineers, 91(1):112-126; Wiklund, D. and Liu, D. 2003. “SoCBUS: Switched Network on Chip for Hard Real Time Embedded Systems,” in Proceedings of the 17th International Symposium on Parallel and Distributed Processing (IPDPS '03), Los Alamitos, Calif., Institute of Electrical and Electronics Engineers Computer Society, page 78a), which communicate with the higher node or core programmed to function as master or ‘supreme’ node or controller, and if pertinent, directly with one another.

The distribution of control between the brain and subordinate circuits and ganglions a salient feature of the nervous system, such a hierarchical scheme may be seen as analogous to the relation between the motor cortex and subsidiary or more localized control circuits in the spinal cord, for example. Here such a control tree receives feedback from the sensors associated with each jacket to continuously adjust and coordinate the dosing of the drug delivery program in detail, and overall.

Subordinate or ‘intimal’ nodes closest to their respective sensors feed into a channel of control directed to one morbidity among morbidities, an organ, organ system, lesion, or midus, the sensor readings used by the master node to apportion the actuation of nondrug therapeutic components such as electrostimulators and the release among these targets of the fewest drugs in the smallest doses as is best likely to reinstate homeostasis or overall health to the extent possible.

The automatic adaptive response hierarchical control system consists of local microcontrollers—usually positioned within the ductus side-entry and impasse jackets and nonjacketing side-entry connectors which represent the subordinate hierarchical levels assigned to the morbidities. These take sensor inputs, which to minimize dissection and achieve the maximum compactness, usually incorporated into the local jacket or connector—and coordinate these within their respective subsystem, or channel of morbidity control, typically assigned to an organ- or organ system-defined channel of morbidity depicted in FIGS. 4 and 5.

Each morbidity is assigned a channel or arm of control in a hierarchical control system which takes sensor inputs at the lowest level. The inputs are passed for coordination to a control node, an intermediate chip-microcontroller, thence to a next higher node which coordinates treatment with its counterpart or counterparts in the other control channel or channels, the number thereof and need for coordination among these dependent upon the number of distinguishable morbidities.

As to route of administration, drugs are infused, that is, released directly into the vascular tree, usually into the supply artery of the organ or tissue volume targeted. In advanced and more complicated use demanding the treatment of complex comorbid disease where the dose of each drug to be delivered to each of numerous target sites must be timed to take into account the time to onset of each drug as well as the exact dose required within the context of the sum of drugs to bring about that combination of drugs most likely to optimally curb the disease overall.

The ‘inertia’ and delay in affecting some physiological parameters considerably greater than it is for others, depending upon the application, no individual or composite form of control, to include model predictive, fuzzy, and proportional-integral-derivative can be ruled out. In general, using different controllers in each type implanted drug reservoir outlet pump or if necessary, belt-worn power, control, and pack is more costly than is the use of a standard microcontroller and development environment; nevertheless, provided simple applications and embodiments prevail for a given type pump-pack, the smaller cost of a simple or hobby grade controller is preferable.

Hierarchical control has been available for decades; however, with no means for safely converging with ductus through a secure junction, the relation of hierarchical networked feedback to automatic drug delivery has remained elusive. Because the sensors are associated with collocated means for the targeting of drugs to the location respective of each, immediate and automatic remedial drug delivery, not just information as to the status of the patient, are obtained.

The penultimate nodes in the hierarchy pass their outputs to a master node microprocessor. By analogy with the nervous system, such can be characterized as the system sensory function. Overall coordination of drug delivery (or other therapy, such as the application of heat), analogous to motor function, is then governed by the master node, a microprocessor which coordinates the inputs from the nodes next lower in rank and passes control signals down the channel in a motor sense, causing implanted drug reservoir outlet valves or pumps to release the drugs as directed.

The overall consequence of the combination of drugs released or other therapy applied such as electrostimulatory or thermal entered into memory, the system ‘learns’ the best combination at a given time and adapts to changes with time, this pattern having diagnostic and prognostic value. Moreover, if automated, the system is able to edit its own prescription-program originally prepared by the pharmacist programmer and thus maintain the optimal time-adjusted therapeutic response to treat the component morbidities or lesions.

A ductus or impasse side-entry jacket equipped with the required electromagnets will draw any sufficiently magnetic field susceptible particle-bound drug outward and through the surrounding lumen wall. The position of the side-entry jacket thus targets the level along the ductus, and the magnetic force targets the intramural lesion in that segment. A lesion such as an atheroma is therefore ‘washed over’ and penetrated by the drug, which can be released continuously or at intervals throughout the day.

Local and Systemic Implications of Automatic Sensor-Driven Targeted Drug and Electrostimulation Delivery

Symptoms even appear in bodily systems that would seem qualitatively unlike and remote from that of origin. Regional enteritides can induce arthritis. Osteoporosis and Paget's disease of bone (osteitis deformans), for example, are disorders often secondary to endocrine disease that affect the skeleton. If arterial applications are stressed, it is because of the disproportional involvement of vessels in death from disease. Bodily conduits are not analogous to inert plumbing; in fundamental contrast to synthetic tubing, the walls of vessels, lymphatics, hormonal ducts, and gut are integrated into a hierarchy of negative feedback loops that to adaptively interact with the passing contents, extend from individual cells to the brain (see, for example, Jameson, J. L. 2005. “Principles of Endocrinology,” in Harrison's Principles of Internal Medicine, New York, N.Y.: McGraw-Hill, page 2072).

When a bodily conduit is itself diseased, effective and efficient treatment requires that medication be actively drawn into, not merely pass through the line. Allowing the medication to pass lesions wastes medication that if targeted would have contributed to an effective dose, exposes healthy tissue downstream to the wasted dose, results in complications, and increasing the dose to increase absorption only increases the waste and the risk. When the supply zone or territory or downstream segment becomes diseased, the contents passing through the line should be adjusted or supplemented to promote healing. While therapeutic agents are often best restricted to frankly diseased tissue, the pathways in which the affected tissue participates, and therewith, the far-reaching relations of that tissue to other tissue, means that the propagation of disease from that tissue to other tissue is not so restricted.

For example, the central negative feedback loops that govern angiotension flow along the hypothalamic-pituitary-adrenal axis. The central loops incorporate, integrate, and drive subsidiary loops more and more local in level down to the individual cells. Tied into the neuroendocrine and autonomic nervous systems, the hypothalamic-pituitary-adrenal axis responds to systemic blood volume by continuously regulating the blood serum levels of steroid hormones produced in the adrenal cortex, such as cortisol, and in the kidney, such as angiotensin II. Angiotensin II directly effects vasoconstriction and secondarily effects the release of aldosterone to regulate the balance between sodium and potassium in the blood, thus enlisting osmolar support to regulate water retention.

Collaterally, vasopressin, or antidiuretic hormone, produced in the hypothalamus and released by the pituitary gland in response to a decrease in blood volume exerts a pressor effect and acts as a diuretic by reducing the volume of urine, thereby conserving the volume of blood. That the caliber of blood vessels, for example, is adaptive locally as well as systemically demonstrates that control is effected by a hierarchy of control loops wherein those subordinate interact with those progressively more encompassing until the center just above the brainstem is reached. Central mechanisms initiate the release of circulating vasoconstrictors or vasodilators that cause the linings of blood vessels to contract or relax in response to the condition of the circulatory system, which includes cardiac output, partial pressures of oxygen and carbon dioxide, and the existing concentration of hormones and electrolytes in the blood.

The system is ambulatory and functions around the clock without control by the patient, who may be asleep. In an emergency not programmed for response distant from the clinic, a preplaced pump-pack can transmit the emergency signal to be activated by remote control. The jackets to be described can be placed in encircling relation about ductus along the digestive and/or urogenital tracts, the vascular tree, and/or the airway, to form a continuous passageway through the lumen of a synthetic line and into the native lumen without significant leakage or trauma and with no portion of the junction endoluminal, or projecting into the lumen. This capability has implications for the treatment of disease on a continuous, automatic, sustained, and when necessary, immediately adaptive basis.

This because the body consists of tissue pipelines and the tissues these supply. Except for absorption through the skin and oral mucosa, all intake into the body is through ductus. Any tissue can be accessed through ductus; when a side-entry jacket can be placed at a level that substantially excludes other tissue, the tissue that will be supplied is effectively isolated for targeted delivery of medication. Moreover, because the wall surrounding ductus support many biochemical interactions and discharge sensory feedback signals that modulate the control of numerous functions, the ability to circumscribe only a certain segment along a ductus for the delivery of drugs can have significant physiological implications, the more so when that segment is diseased.

In addition to communication affected by the autonomic nervous system, the luminal wall can release signaling proteins, such as chemokines and interleukins, and the luminal contents can include enzymes, hormones, cells containing cytokine signaling proteins, and so on, so that remote tissues are affected as well. As a result, there is no disease in which bodily conduits are uninvolved. No bodily conduit is analogous to inert plumbing; all are integrated into a hierarchy of negative feedback loops from the individual cells to the brain to actively and appropriately interact with the constitution, pressure, and velocity of passing contents.

The atrial walls, aortic, and carotid sinus bodies (glomus caroticum, carotid glomus) contain chemoreceptors that detect blood gas and acidity levels, which transmitted to the medulla, signal the autonomic nervous system to adjust the respiratory and heart rates and the stroke volume. Similarly positioned baroreceptors, or pressoreceptors, detect the blood pressure, likewise transmitted to the brainstem, which regulates subsidiary feedback control loops. Placed along an artery, the level at which simple junction jackets such as those shown in at the top of FIG. 4 and described below are positioned sets the supply territory or region.

Advancing the jacket along the artery toward its end supply excludes more proximal branches to neighboring tissue, closing in upon and so narrowing the target zone or supply territory. By the same token, retreating along the artery admits side branches to neighboring tissue, thus expanding the zone. The junction bidirectional, antegrade delivery into the native lumen, whether vascular, digestive, urinogenital, respiratory, for example, is usually of a drug, whereas retrograde delivery from the lumen is usually of a diagnostic test sample.

Accordingly, automated ambulatory systems of pumps able to individually deliver any of a number of different drugs to jackets placed at different levels along a single ductus, different ductus belonging to the same bodily system, or ductus belonging to different bodily systems according to a programmed schedule and mediated by sensor implants have the potential to treat morbidities and comorbidities in a discretionary manner whereby each drug is delivered to the target tissue in a time coordinated sequence. Such treatment has the potential to outstrip any therapy dependent upon the systemic, hence, necessarily indiscriminate, administration of drugs. Susceptible to primary disease, and supplying and draining every part of the body, the treatment of bodily conduits has application to any localized condition.

Drug delivery through a side-entry jacket allows the upstream ductus and tissue it supplies to be avoided. When more effective, the drug can be increased in concentration for the target tissue while substantially reduced in dose compared to the systemic dose that would be needed to achieve the same dose at the target. Whether through the use of a reversal agent or an extraction-jacket, as will be described, if necessary any residue of the drug can be truncated from further circulation at a segment cutoff level. When a bodily conduit or ductus (singular) is itself diseased, effective and efficient treatment requires that medication be actively drawn into, not merely pass by it through the lumen with little uptake.

For disease within the wall of the ductus itself, the junction is extended to incorporate a magnetic collar of which the field strength is incrementally increased in the antegrade direction to achieve a more uniform penetration. Mechanically and magnetically based, the drug targeting spoken of here averts the contingency of discovering a substance that depends upon intrinsic properties and affinities for targeting therapy at the gross anatomical level. A drug must, for example, inhibit a destructive enzyme produced as the result of a genetic defect, such as the tyrosine hydroxylase inhibitor imatinib mesylate (STI-571; Novartis Gleevec®) to selectively target cancer cells.

Or it must take advantage of an inherent affinity of an organ or gland for a substance, such as the thyroid gland for iodine. Here instead, the drug is contained while conducted to the treatment site, where it is forcibly drawn into the surrounding tissue, regardless of its inherent proclivities. Allowing the medication to pass lesions within the wall surrounding the lumen, or ductus-intramural lesions, without uptake wastes medication that if targeted would have contributed to an effective dose, exposes healthy tissue downstream to the wasted dose, and results in complications.

Moreover, increasing the dose to achieve better absorption only increases the waste and the risk. Drug targeting substantially limits exposure to the drug to the tissue intended, isolating the drug from other tissue targeted elsewhere in the body by the same control system. This makes it possible to target a transplant organ without exposing the entire body to immunosuppressive or immunomodulatory medication, and can significantly reduce if not eliminate the damage to the immune system done by chemotherapy and radiation, for example.

The value of drug targeting with respect to the administration of immunosuppressive drugs, nonsteroidal anti-inflammatory drugs such as aspirin, which used to treat arthritis, for example, often produce gastritis and ulcers, statins that induce myositis in susceptible patients, steroids which can produce moon facies and induce diabetes, for example, and the avoidance of adverse side effects, drug-drug and drug-food interactions that would be caused by piped drugs were these not isolated across the entire array of pharmaceuticals. All drugs bode potential complications, making directly piped targeting significant in eliminating such adverse sequelae (see, for example, Polyak, B. and Friedman, G. 2009. “Magnetic Targeting for Site-specific Drug Delivery: Applications and Clinical Potential,” Expert Opinion on Drug Delivery 6(1):53-70).

Conventionally, magnet implants are limited to permanent magnets used to secure dental and maxillofacial prostheses and cochlear implants, and implanted rings used to ligate and atrophy tissue by compression ischemia. Other applications of magnetism require the use of an extracorporeal electromagnet to direct the magnetic field toward the treatment site, which limits such use to the clinic. The importance of drug targeting with respect to preventing rejection in transplantation, for example, will be addressed. Drug targeting can also be of value in averting side effects that preclude their use due to drug intolerance.

Jacket placement assumes that the medication will be required on a long-term basis, would best not be taken orally, or by injection, or injection that must be frequent as would promote patient noncompliance, and that accessibility to the site in order to implant the jacket and a port at the body surface to be described will not result in trauma more than negligible and transient. Direct access to or into any internal structure otherwise inaccessible from outside body except through an invasive procedure limited to the clinic and achieved instead from outside the body by electrical control makes impediment-free treatment available at any time.

With intractable and painful dysuria such as caused by interstitial cystitis, for example, the direct pipeline targeting into the bladder without the need for invasive instillation of ameliorative medication such as 100 milligrams of pentosan, 80 of lidocaine, 3 ml of sodium bicarbonate, and a nonsteroidal anti-inflammatory, for example through the body wall (The Merck Manual, 2006, page 1962), is a significant improvement in patient quality of life.

When the dosage regimen frequent, and/or multiple drugs are needed making self-administration problematic, drug delivery is not dependent upon patient compliance but rather automatic as programmed, through a direct catheteric pipeline to the jacket or through plural lines respective of plural jackets from a port implanted at the body surface. At the same time, the port is available to administer another drug in the clinic from a syringe, for example. In order to realize the benefits of drug targeting over a period of years if not life, it is essential to possess means for establishing secure connections to ductus. The long-term indwelling of a catheter, needle, endoluminal implant, or prosthesis in a vessel often leads to adverse complications.

Subclavian, femoral, and internal jugular lines, and even peripherally inserted central catheters or PICCs, for example, are susceptible to infection, occlusion, breakage, and leaks (see, for example, Jumani, K., Advani, S., Reich, N. G., Gosey, L., and Milstone, A. M. 2013. “Risk Factors for Peripherally Inserted Central Venous Catheter Complications in Children,” JAMA Pediatrics 167(5):429-435; Barrier, A., Williams, D. J., Connelly, M., and Creech, C. B. 2012. “Frequency of Peripherally Inserted Central Catheter Complications in Children,” Pediatric Infectious Disease Journal 31(5):519-521; Shen, G., Gao, Y., Wang, Y., Mao, B., and Wang, X. 2009. “Survey of the Long-term Use of Peripherally Inserted Central Venous Catheters in Children with Cancer: Experience in a Developing Country,” Journal of Pediatric Hematology and Oncology 31 (7): 489-492).

Due to the risk of injury, air embolism, or the formation of a hematoma, maintaining multiple such diagnostic sampling and/or drug delivery points in different veins with indwelling catheters is not feasible, certainly not in an ambulatory patient, much less in one who is very young or very old. Moreover, even though direct access to the blood supply to an affected organ or region would afford considerable advantages both diagnostically and therapeutically, this cannot be done with respect to small much less major arteries, wherein the blood pressure is greater. However, the ability to form several secure junctions with arteries, even large ones, opens the way for targeting medication to, taking draws from, and inserting a diagnostic probe into the blood supply of the organs or tissues these supply.

Extended Capabilities

For less power demanding applications, power is obtained by carrying charged button cell batteries to replace the one or more in the surface port. Higher demand on a continuous basis calls for a larger implanted rechargeable battery, the surface port then used to take power from an electrical outlet. The need for more power on an intermittent basis can be satisfied by connection to an external power source with or without recharging a battery. Transdermal energy transfer allows direct tetherless delivery of power whether a battery is simultaneously recharged within a circumscribed area.

Most applications of ductus side-entry connection jackets simple and direct, a secure means for forming a junction with a ductus allows the application of a body area network with wireless transmission, or telemetry, even combined with transdermal energy transfer (see, for example, Mao, S., Wang, H., Zhu, C., Mao, Z. H., and Sun, M. 2017. “Simultaneous Wireless Power Transfer and Data Communication Using Synchronous Pulse-controlled Load Modulation,” Measurement (London, England) 109:316-325; RamRakhyani, A. K. and Lazzi, G. 2014. “Interference-free Wireless Power Transfer System for Biomedical Implants Using Multi-coil Approach,” Electronics Letters 50(12) 853-855; Yazicioglu, R. F., Torfs, T., Penders, J., Romero, I., Kim, H., and 4 others 2009. “Ultra-low-power Wearable Biopotential Sensor Nodes,” Conference Proceedings, Institute of Electrical and Electronics Engineers Engineering in Medicine and Biology Society 2009:3205-3208; Yoo, H. J., Cho, N., and Yoo, J. 2009. “Low Energy Wearable Body-sensor-Network,” Conference Proceedings, Institute of Electrical and Electronics Engineers Engineering in Medicine and Biology Society 2009:3209-3212; Young, D. J. 2009. “Wireless Powering and Data Telemetry for Biomedical Implants,” Conference Proceedings, Institute of Electrical and Electronics Engineers Engineering in Medicine and Biology Society 2009:3221-3224; Panescu, D. 2008. “Wireless Communication Systems for Implantable Medical Devices,” Institute of Electrical and Electronics Engineers Engineering in Medicine and Biology Magazine 27(2):96-101; further references provided below) to afford immediate diagnosis and targeted drug delivery at multiple locations under automatic control.

In such a hierarchical control system, the processors of a monolithic integrated circuit or microchip multicore microcontroller are partitioned to support one control node each, that programmed to function at the highest level of control as the master node sent the inputs from and having a time horizon comprehensive of the subordinate nodes, of which each contributes inputs to the pumps and jackets of the set based upon the sensors that feed it. Various arrangements possible, separate microcontrollers can be assigned to each node in the control tree.

The object is to optimize drug delivery while least interfering with freedom of movement. The sensors, the positioning of these, and the control desiderata among the nodes signaling a pump-pair depend upon the disease under treatment and vary widely. Once the implanted elements have been placed, only the need to replenish one or more drugs by injection through the body surface port and into a pectoral reservoir or replacement of the vial in the pump pack interrupt the patient in free movement.

Much as a vaccine confers artificially acquired immunity, such a system effectively serves as an adjunct or nonintrinsic suppressive or negative feedback response loop for adapting to an anomalous condition. By comparison, automatic ambulatory insulin pumps deliver insulin subcutaneously, intramuscularly, hence, systemically following a time delay, without targeting ability, and intravenous drug delivery is unsuited to an active life. Implant cardioverter defibrillators deliver electrical current, not fluid drugs, and ventricular assist devices provide mechanical action. Many genetic defects result in a failure to produce an essential enzyme or protein, or to produce the substance in the normal form and/or amount.

A disorder response system that supplements or substitutes for a defective intrinsic response constitutes what is in effect a physiological prosthesis, whereas a system placed to compensate for a genetic defect that evokes no innate adaptive mechanism is bionic. An automated prosthetic disorder drug delivery response system can function as a backup immune system to compensate for deficiencies in intrinsic adaptive responses, and where an intrinsic response is not just deficient but entirely lacking, such as where due to an inborn error in metabolism an essential enzyme is not produced, the automatic disorder response system can be characterized as bionic.

The distribution of control between the brain and subordinate circuits and ganglions a salient feature of the nervous system, such a hierarchical scheme may be seen as analogous to the relation between the microprocessor as master controller and the cortex and subsidiary or more localized control circuits in the spinal cord, for example. Here such a control tree receives feedback from the sensors associated with each jacket to feed data up the hierarchical levels continuously to adjust and coordinate the dosing of the drug delivery program both in detail, and overall.

While ground level subordinate or ‘intimal’ nodes associated with ground level sensors feed into a channel of control usually dedicated to one of plural morbidities, the same approach can be applied to organs, organ systems, lesions, or nidi. In any case, once processed by the subordinate nodes, or controllers, and the sensor readings have been passed up to the master node to apportion the release of drugs and/or other therapeutic measures among the targets, the consequence should best approximate normal homeostasis across the targets for the patient.

Homeostatic optimization for a specific patient means that if present, deficiencies and defects of anatomy or physiology can prevent the realization of optimal homeostasis that would be possible were such obstacles absent. In a tertiary medical center with the patient stationary, this scheme can be expanded so that diagnostic sensor feedback initiates and regulates not only ongoing dosing from among clinician prescribed drugs loaded, but can select as well as deliver drugs from among an unlimited number of drug supply reservoirs. Numerous genetic defects result in metabolic disorders, due, for example, to a failure to produce an essential enzyme or protein or to produce the substance in the normal form and/or amount.

Notable examples include phenylketonuria, alkaptonuria, pyruvate dehydrogenase deficiency, Lesch-Nyhan syndrome, Kearns-Sayre syndrome, Zellweger syndrome, Gaucher's disease, Niemann-Pick disease, glycogen storage diseases, galactosemia, organic acidemias, urea cycle defects, long-chain hydroxyacyl dehydrogenase deficiency, intolerance to sugars such as lactose or fructose, and many others (see, for example, Fong, C.-T. 2006. “Inherited Disorders of Matabolism,” in The Merck Manual 18th edition, section 19, chapter 296, pages 2458-2475; Rader, D. J. and Hobbs H. H. 2005. “Disorders of Intermediary Metabolism,” in Harrison's Principles of Internal Medicine, part 14, section 3, pages 2286-2298).

A fully implanted automatic drug delivery system can detect the instant shortage for such a substance or substances and provide these in the correct amount without the patient being aware of it, making this ability especially beneficial in patients such as the very young, demented, and uncooperative, who cannot be depended upon to adhere to a prescription.

While the drugs delivered must be compatible, which is readily accomplished when delivery is targeted, such a system seeks to detect and return diagnostic information, such as at the level of metabolites, antibodies, antigens, and organic or inorganic substances, in relation to homeostatic balance without necessarily ascribing combinations of imbalances to a particular syndrome. Fully implanted and power and drug pack portable systems are loaded with a set of specific drugs to treat a diagnosed or predictable condition. To accommodate unpredictable as well as predictable eventualities, one factor deciding which drugs to store is broad spectrum, the applicability to treat an array of disease conditions.

By contrast, a stationary system need not be limited thus and does not require a preestablished diagnosis, so that correction expeditious, the risk of misdiagnosis is less. The direct delivery of drugs without relationship to a specific diagnosis allows immediate response to reasonably predictable intercurrent disease, especially valuable when comorbidities are likely. For example, with no change in behavior, metabolic syndrome, or the combination of abdominal obesity, hypertriglyceridemia, lowered high-density lipoprotein serum level, elevated plasma fasting glucose and low-density lipoprotein levels, and hypertension, progression to diabetes and cardiovascular disease is predictable, but not as to time of onset.

Such represents the internalization and rendering immediate of point of care detection (see, for example, Chikkaveeraiah, B. V., Bhirde, A. A., Morgan, N. Y., Eden, H. S., and Chen, X. 2012. “Electrochemical Immunosensors for Detection of Cancer Protein Biomarkers,” ACS [American Chemical Society] Nano 6(8):6546-6561; Rusling, J. F. 2012. “Nanomaterials-based Electrochemical Immunosensors for Proteins,” The Chemical Record 12(1):164-176; Rusling, J. F., Kumar, C. V., Gutkind, J. S., and Patel V. 2010. “Measurement of Biomarker Proteins for Point-of-care Early Detection and Monitoring of Cancer,” The Analyst 135(10):2496-2511; Choi, Y. E., Kwak, J. W., and Park, J. W. 2010. “Nanotechnology for Early Cancer Detection,” Sensors (Basel, Switzerland) 10(1):428-455. Liu, G. and Lin, Y. 2007. “Nanomaterial Labels in Electrochemical Immunosensors and Immunoassays,” Talanta 74(3):308-317).

For such patients with both portable and stationary systems, prepositioning sensor implants to detect and loading the stationary dispensing system with drugs to treat the additional symptoms associated with congestive heart failure, for example, allows the system to respond to these additional symptoms upon onset. Large in number, with additional drugs appearing often, the complement of drugs dispensed by such a stationary system is reduced to those for each purpose which clinical trials have shown to be safe and effective. The automatic drug selection and delivery control program or prescription data switches the drug reservoir catheters connected to each target ductus from among an unlimited number of drug supply reservoirs. In this, a body area network under ‘intelligent’ complex or hierarchical adaptive control can also be made to transmit data through a wireless network.

More than a single subordinate control level exceptional, a pump-pair and jacket set that includes three jackets, for example, requires a microcontroller with at least four cores (referred to by Parallax, Inc., whose multicore microtroller chips have the individual cores arranged in a circle or ‘hub’ for access to shared memory, ‘cogs’). Magnetic gradient-incorporating side-entry jackets, or piped impasse-jackets, already capable of drawing superparamagnetic carrier bound drugs radially outward through a ductus wall, patch-magnets are placed not to encircle ductus, but attached to the outer capsule of an organ supplied by the ductus and subject to the disease process under treatment. In most instances, the sensors are packaged in the form of stays configured for concentric insertion into the wall of the ductus or parenchyma before the jacket or patch-magnet is applied, so that these sit beneath or within the jacket or patch-magnet.

Minute diagnostic sensor implants respective of each jacket, patch-magnet, or other type implant provide feedback to the lower level nodes respective of each jacket, patch-magnet, or other type implant feedback site, thereby adjusting the dose of the drug respective of each within the prescribed drug delivery context, or the prescription as maintained by the master node. Highly stable conditions may require no more than one sensor closed feedback loop if any. Significant cost reduction may be achieved by limiting control software and hardware to the nonadaptive where more complex control and artificial intelligence are unnecessary. Whether control is nonadaptive or complex, the drivers remain standardized interchangeable pump-pair and jacket set open loop-driven stepper motors. The program automatically and immediately adjusts the delivery of medication for the present condition.

To cover different ranges of disease severity, the multicore microcontroller stores more than one program or prescription. Upon receiving appropriate sensor feedback through one or more subordinate nodes, the master node automatically transfers the program for the out of range node and jacket or the entire set. Should the feedback signals reflect a condition outside the drug delivery response range of the apparatus, a wireless body area network transmits an alarm to the clinic by emergency band or a conventional communication means, such as a text message. Depending upon the urgency, the input can be applied to dispatch an ambulance, alert the patient to return to the clinic, or instruct the patient to connect the pump-pair intake turret lines to higher capacity tabletop drug reservoirs containing the same or different drugs and switch to a different prestored control program.

The set produced as a unit, a single jacket set omits pump-pair outlet turrets, while multi-jacket sets with a reasonable limit of four jackets provide pump outlet turrets to allow switching the pump outlets to any one jacket at any one time. More elaborate line switching as would permit simultaneous outlet switching to more than a single jacket at a time is possible but elusive of practical medical purpose, needlessly complex and costly, and inviting human error. In any such set, the lines connecting the pump-pair to each jacket is permanently fastened to the main and sidelines of each jacket, pump outlet switching among jacket inlets in the set accomplished at the pump outlet turret where any line to any jacket in the set can be rotated into alignment with the pump outlet.

A pump or pump-pair and jacket set thus constitutes a unit apparatus, of which portions proximal to the port implanted at the body surface remain outside the body, or extracorporeal, with those distal to the port implanted, hence, intracorporeal. Since individual jackets in a given standardized pump-pair and jacket set can be different sizes, can be placed along different type ductus in different parts of the body, and the one pump-pair supporting the jacket set allows the delivery of any drug to any jacket in the set in any sequence at any time, to further admit the inter-switching of lines among different pump and jacket sets only causes confusion. Jackets belonging to different pump-pair and jacket sets can be interposed with drug delivery times controlled by the multicore microcontroller in the multipump-pair power and control housing, or base into which the pump-pair plug-in modules insert. However, the need for more than one such set should prove rare and limited to cases of severe multiorgan disease or extensive injury.

The implanted system of catheters and ductus and tissue connectors to target drug delivery requires prosthesis-to-native tissue junctions which will remain secure and to the extent possible, conform to neighboring structures in a compliant manor without becoming disoriented. Other attributes essential for long term sufficient service include insusceptibility to the development of leaks, microbial intrusion, or injury to the substrate ductus or tissue to which the connector is mounted. These connectors must also be maintainable by means of inmate service or accessory channels that allow direct access without the need for reentry, even endoscopically.

Specifically, connection for securely and least disruptively merging catheteric drug and blood pipelines, or druglines and bloodlines, and native lumina is described in copending nonprovisional application Ser. No. 14/121,365 entitled Ductus Side-entry Jackets and Prosthetic Disorder Response Systems, filed on 25 Aug. 2014 and its continuation-in-part Ser. No. 15/998,002. This application was directed to the creation of passages between synthetic and native ductus and the reverse by means of dependable connectors and durable connections able to remain in place indefinitely without damage to the substrate ductus, and if necessary, adapt to growth over a period of years.

Another application, Ser. No. 14/998,495, now U.S. Pat. No. 11,013,858, entitled Nonjacketing Side-entry Connectors and Prosthetic Disorder Response Systems, also addressed means for securely fastening the distal ends of catheteric pipelines, injection needles, electrodes, and miniature diagnostic and therapeutic probes, for example, to the surfaces of nonductal tissue such as that of the solid organs and glands for an indefinite if not lifelong treatment of chronic disease. As must ductus side-entry jackets, side-entry connectors must provide a synthetic-to-native junction which is durable, positionally stable, will accommodate growth, remain durable and leak-free, allow the direct delivery of drugs into the connector nor induce atheromatous degeneration in the substrate ducts, such a system could not remain implanted over more than a brief period in the clinic.

For a sustainable and durable ambulatory automatic response system, such connectors are a central prerequisite. Both of the applications cited delineated the assignment of the axes of control in a fully implanted hierarchical control system to different morbidities, organs, or organ systems in the treatment of comorbid disease. The term ‘comorbid’ is intended to denote coexisting disease conditions whether or not these are directly related or cooriginal. Nonjacketing side-entry connectors extend this capability to structures such as the heart, stomach and colon, which abruptly motile and large in diameter, need not be jacketed or collared, as well as to nonductal tissue, prompting revision of the title from ‘nonductus’ to ‘nonjacketing.’

The junction created may be conventional and singular or support one in a number of disease process treatment control axes or channels of an automatic ambulatory prosthetic disorder response system placed to act as a backup ‘immune’ system.’ Autonomic motor assist devices mentioned in passing are deferred for full description in an application to follow, that present concerned with electrical and pharmacological applications of nonjacketing side-entry connectors.

Such a lower to higher levelled control system is an adaptive ambulatory hierarchical prosthetic disorder response system, distinct from a conventional system such as a continuous glucose monitor which injects insulin as the need therefor is detected. With such a rudimentary device, the injection of insulin remains intramuscular and its dispersal systemic. In contrast, a system of the type meant is fully implanted, and the insulin is not dispersed but released through a side-entry jacket directly into the portal vein as would a normal pancreas.

This eliminates the need for the intramuscular injection of an insulin, with a time lag that compared to release targeted thus is considerable. No subcutaneous injection, oral antihyperglycemic drug, or inhaled formulation of insulin, metformin, or any other drug can approximate the ability of an instant response system with multisensor input under hierarchical control to modulate blood glucose to within the normal range. Insulin overdose or overproduction should it arise is remediable by releasing metalloprotease insulin-degrading enzyme (insulysin, insulinase) or glucose directly into the hepatic portal vein or glucose into the bloodstream.

Because the automatic disorder response system is equipped and programmed to maintain its own components as well as to monitor and treat the disease, provided the drugs required if any are replenished as necessary, and except for periodic charging, usually by means of transdermal energy transfer, it is meant to function autonomously for years. To treat symptomatically complex comorbid disease, which may elude diagnosis, such a negative feedback system assigns lower level closed loops to the control of individual symptom values, such as characterize a key metabolic pathway or process. The conventional treatment regimen established, inputs from symptom or variable sensor implants provide feedback, to which the controller responds by adjusting the delivery in dose level and interval of pharmaceutical and/or electrical therapy to recover to the programmed target set point as the normal value.

Where the regimen is unestablished, different drugs and electrical discharge patterns are first established in the clinic. In the treatment of comorbid disease, higher level control is applied to monitor the summary or overall homeostatic condition and if necessary, apply adjustments among the control axes, such as to shift subordinate set points when necessary. Such a system is fully, or closed-skin, implanted, a belt-worn battery pack such as those used with conventional implanted assist devices required only when the simultaneous treatment of multiple comorbidities creates a demand for power too large to be satisfied by transcutaneously, or transdermally, recharged implanted or body surface port-held button cells.

If and only if necessary, an extracorporeal battery pack is connected to the intracorporeal components through a body surface, or on-the-skin jack, socket, or port, designed to be easily kept sterile, different types described and illustrated in copending application Ser. Nos. 14/121,365 and 15/998,002, entitled Ductus Side-entry Jackets and Prosthetic Disorder Response Systems, FIGS. 27 and 28 and Ser. No. 16/873,914. entitled Vascular Valves and Servovalves—and Prosthetic Disorder Response Systems, FIGS. 26A thru 26C.

Where the means for achieving the overall homeostatic condition closest to that optimal, meaning closest to that normal, among an unfamiliar combination of comorbidities is unclear, the level-by-level ‘bottom up’ data buildup of the self-optimizing and diagnostic empirical approach applied by the control system allows it not only to treat and diagnose a complex condition but to actively and methodically seek out and depending upon the combination of drug onset times, eventually define this condition. With such a system, this process of self-optimization proceeds without interruption until resolved, undegraded by lapses between blood draws or other intermittent tests and unconstrained by limitations of time and staffing in a clinic.

The system functions continuously through stress testing, to include iatrogenic to ascertain the relative value of different drugs under such conditions. More broadly, the system can be programmed to determine and report the optimal dose of any drug previously not deliverable as isolated by direct pipeline targeting. A lucid wearer who documents events such as periods of exercise and sleep can further aid in clarifying the relative performance of different drugs during such periods. In that the detection of unsensed involuntary dysfunction whether of autonomic motor or metabolic function cannot depend upon patient awareness, the dependency upon patient perception to elucidate the nature of the complaint is eliminated.

For this reason, a system that automatically responds to unsensed malfunction, an implanted backup ‘immune system,’ must initiate remedial action immediately on the basis of sensor inputs without the participation of the patient. Unsensed aberrations of physiology must be entrusted to sensor inputs chosen and positioned to detect indicia associated with the disorder or disorders and to implanted electrical, mechanical, and chemical effectors. Side-entry jackets and connectors can fix the position of sensors that would otherwise lack positional stability, at the same time delivering drugs and/or electrical current to the site of implantation.

When the patient is likely to harbor secondary or additional disorders at a later date, an initial procedure responsive to incontinence, just as any to treat a singular disorder, is responded to by initial treatment using componentry that except for placing a body surface rather than subdermally positioned injection port, or portacath, allows the introduction of additional control channels as may later become necessary. If secondary or sequelary morbidity is likely to affect the same region or organ and the connection—unlike one that conveys blood or a drug to be delivered continuously—the different viewing, diagnostic, and therapeutic devices to be used are of like diameter, allowing these to be inserted interchangeably through the aperture at the center of the nonjacketing side-entry connector.

Depending upon the individual patient and the number of drug delivery components likely to become necessary at a later date, a tissue expander to create more space for the small flat drug reservoirs can be placed in the pectoral or pelvic regions, for example. The drug delivery components, include a subdermal surface port, catheteric pipeline from the port to a drug reservoir with outlet pump controlled by the microcontroller, or in comorbid disease, the microprocessor, which to allow the retrieval of biopsy test samples, can be reversible, and the terminal connector—ductus side-entry jacket or nonjacketing side-entry connector to the tissue to be targeted.

Of these, when larger in number, the drug reservoirs are preferably retained within one or more surgically constructed pockets positioned subdermally in the pectoral or pelvic regions. An object of good design being to spare the patient the impediment of a belt-worn pack, intracorporeal positioning thus, provided it does not impose undue discomfort for the patient, is preferable. In a more elaborate system to treat several comorbidities, the need for an extracorporeal belt-worn pack may be unavoidable, so that in this situation, the drug reservoirs can be housed within the same enclosure as the batteries that serve as power source. To assure its immediate sighting, the free proximal end of the catheter (line, feedline) is crimped with a magnetically susceptible ferrule marked with contrast, such as tantalum-based.

If the number of drugs to be provided for the same or different disease necessitates, an external port with multiple openings, each clearly marked is used. Prepositioning a connector with piping and electrical conductor or conductors and control electronics—but not a portacath, reservoir, or pump, which can be placed later—allows testing electrostimulation as the first and best option. Not requiring a portacath, reservoir, or pump, for example, electrical means involve the fewest components, take up the least space, and generally allow placement with the least dissection. Provided unintended function of like innervation is unaffected, the lead or leads can be positioned at a functionally and anatomically higher level. A sacral neuromodulator, for example, may exert an effect on rectal as well as bladder function where only one or the other called for treatment.

In such a circumstance, more highly resolved stimulation farther along the neural circuit once the nerve divides to send the target organ its respective branch or ramus is therapeutically selective in eliminating unwanted concurrent stimulation of another organ, such as the rectum where the bladder had been intended. Then, however meticulous was the testing before implantation, even tiny movement of the lead, whether tined or barbed, cannot shift the distribution of stimulation. Using an electrode, lead, or leads to stimulate the innervation, electrostimulatory neuromodulation is least invasive of a native sphincter, and unsusceptible to the complications associated with pharmaceutical treatment, to include adverse side effects, drug-nutrient, and drug-drug interactions. When the likelihood of secondary disease is high, the need to reenter is best avoided by prepositioning the additional components that would become necessary to treat these.

At the same time, including at the outset fluid, meaning drug, blood, or urine catheteric pipelines that may later become necessary allows for the addition of other components as necessary without the need to reenter the patient in order to add or replace the connector or to place additional lines at a later date. Initial placement best enables the delivery of treatment beyond that contemplated at the outset, not just to allow adjustment in a single therapeutic modality but in the modality or combination of modalities. The concept of making it possible for the therapy to be adjusted without the need to revise the initial procedure or replace the original implants at a later date applies not just to disease able to induce sequelary pathology but to specific disorders for which the best therapeutic regimen will need to be adjusted, as well as when the optimal result can be found only through empirical testing.

To cite one instance, with a refractory gastric reflux that resists treatment with a proton pump inhibitor and/or induces unwanted side effects at the oral (systemic) dose necessary, delivery through a side-entry jacket or nonjacketing side-entry connector at the lower esophageal (cardiac, gastroesophageal sphincter, at the gastroesophageal junction allows the dose to be increased to a level that if circulated could result in anchlorhydria, or an insufficiency of hydrochloric acid in the gastric juice as is necessary for the normal breakdown of food and digestion. Copending application Ser. No. 14/998,495, entitled Nonjacketing Side-entry Connectors and Prosthetic Disorder Response Systems, showed that the semicircular needles used to securely anchor the connector into the substrate tissue could be electrified in a number of different electrical discharge patterns.

This feature afforded the ability to vary the delivery of current concomitant with the piped delivery of different drugs. If the decision is made to resort to electrostimulation of the sphincter, in lieu of or in combination with medication, the connector, already in place, can be used to test numerous modes of electrical pulsation or drug based treatments with or without concurrent or intermittent electrostimulation. The ability to directly target familiar drugs, hormones, and enzymes allows the use of these in novel ways that can advance pharmaceutical science equivalent to the development of new drugs.

Where a different etiology would reasonably effect these different component modes of treatment in a distinctively different way, existing electrostimulators, or neuromodulators, such as sacral and gastric are limited to electrostimulation of the innervation; usually at a high enough level as to involve unintended tissue.

Conventionally, diabetes, which affects the entire body, is treated separately, whereas here, the systemic therapy is certainly provided but also locally coordinated with means to remediate the local consequences of the systemic disorder. Damage to the vagus nerve may be uninvolved in some gastroparesis, or may have resulted in other damage to the stomach, so that only to electrostimulate the nerve would never afford a cure. In fact, the condition is usually treated pharmacologically as well, but without the benefit of direct pipeline targeting which eliminates constraints of dose.

In most cases, the more detailed components of the condition will not be known; however, empirical adjustment to determine the optimal combination of electrostimulatory and pharmaceutical curative factors will not only serve to more effectively ameliorate the medical problem but help to explain its basis. In this process, the fact that the electrostimulation and drugs are precisely targeted eliminates the host of detractive factors contributed by exposure to the drugs and electrostimulation of extraneous tissues and organs. The pathophysiological analysis as to etiology and optimal treatment regimen for a given condition in a given patient are hindered by the number of variables, which is only further complicated when extraneous tissue is involved.

Analogous application to dysmotility along the gut or urinary tract is intentional. Complicated conditions may necessitate a coordinated response that addresses collateral conditions elsewhere within the same or in other organ systems. The satisfactory application of a therapeutic regimen which senses the need for and automatically actuates a coordinated response that includes directly targeted electrical discharges and/or drug delivery, as well as autonomic motor assist devices, requires and justifies the placement of a microcontroller, sensors, and other components necessary to provide such a coordinated response.

Administered conventionally, proton pump inhibitors taken orally often fail to afford sufficient relief of acid reflux or of gastroparesis, and prokinetic, or promotility, drugs, such as erythromycin, domperidone, metoclopramide, (Camilleri, M., Parkman, H. P., Shafi, M. A., Abell, T. L., Gerson, L. and the American College of Gastroenterology 2013. “Clinical Guideline: Management of Gastroparesis,” American Journal of Gastroenterology 108(1):18-38; additional references cited in U.S. Pat. No. 11,013,858, section entitled Background of the Invention, column 17) which may be injected with an endoscope, have yielded unsatisfactory results for the long term relief of gastroesophageal reflux, as have hormonal and antinausea therapy.

In addition to the increased utility of drugs that must not be administered systemically at a dose limited by the need to avoid adversely affecting other tissues, automatically targeted delivery at intervals of a short duration drug such as botulinum toxin type A to a sphincter, for example, elevates it in utility from a temporary palliative, means of confirming a diagnosis, and possibly averting a surgical procedure to a sustainable source of relief.

It should be assumed that the need for additional druglines will develop over time; especially if the insertion site is deep as will detain revision, fluid lines (catheteric drug pipelines) connected to the side-entry connector are routed to minimize the risk of organ strangulation. At least until the need therefor arises, these should be tunneled subdermally, so that the free proximal ends are prepositioned for connection to drug delivery components once these become necessary. If the number of drug target sites exceeds the number of subdermal ports acceptable, then a body surface type nonjacketing side-entry connector as described in copending application Ser. No. 14/121,365, entitled Ductus Side-entry Jackets and Prosthetic Disorder Response Systems is placed.

Such a surface port can be placed temporarily during the initial drug testing period. If the number of drug target sites is reduced, the external port connecter is removed and replaced by portacaths. The need to continue with an external port connecter is limited to comorbidity that poses numerous electrostimulation and/or drug target sites. Provided the systemic medication previously used provided some relief, the treatment commences with the same drugs, with, however, the dose adjusted for direct targeting, wherewith the exposure of unintended tissue ceases as a consideration.

To be driven forward from a body surface port implanted subcutaneously in the pectoral region, for example, to the target, small amounts of drugs can be placed at the head of a column of water, and can be arranged for intermittent automatic dispensing by varying the length of the water segments between successive doses. The reversible pumps used allow drugs to be withdrawn, the line and reservoir if present flushed clean, and another drug introduced.

The reason that such an assist device with electrical and fluid delivery capability is not placed at the outset is that to encircle the native sphincter requires an extent of circumesophageal dissection and the use of suture to stabilize the surrounding tissue and thus avoid producing the effect of sliding hiatus hernia. Primarily to prevent migration, autonomic motor assist devices such as an electromagnetic sphincter have suture pass-through loops such as those shown as part number 32 in the accompanying drawing figures therein.

These loops, at several points toward the proximal and distal margins, allow connection of the implant to the surrounding tissue, here to the diaphragm, the sphincter and diaphragm therefore moving together. The use of an electromagnetic sphincteric assist device should be viewed as a last resort; in all but a small proportion of cases, the patient would never require mechanical assistance, so that the need for revision would almost always have been avoided.

This action is best stimulated electrically, and next best through the direct application of inotropic drugs through a nonjacketing side-entry connector. An electromagnetic sphincteric assist device does not function thus but applies constrictive force entirely about the native sphincter. Because this mode of constriction is different than that to which the native sphincter is adapted (see, for example, Theodosiou, N. A. and Tabin, C. J. 2005. “Sox9 and Nkx2.5 Determine the Pyloric Sphincter Epithelium under the Control of BMP Signaling,” Developmental Biology 279 (2): 481-490; Moniot, B., Biau, S., Faure, S., Nielsen, C. M., Berta, P., Roberts, D. J., and de Santa Barbara, P. 2004. “SOX9 Specifies the Pyloric Sphincter Epithelium through Mesenchymal-epithelial Signals,” Development (Cambridge, England) 131(15):3795-3804), an electromagnetic sphincteric assist device should always incorporate a fluid line for drug delivery to ameliorate any adverse sequelae of forcible constriction.

A potential disadvantage of conventional electrostimulation is that the stimulation is applied to a larger nerve which intercepted at too high a level is likely to include fibers that will eventually ramify to tissue other than that to be treated. In most instances, a side-entry connector is local to the target tissue, so that affecting unintended tissue is out of the question. Whereas electrostimulators have limited prescribed points of insertion, ductus side-entry jackets and nonjacketing side-entry connectors can be placed at any nervous or vascular level to deliver any combination of electrical discharge and/or medication.

In the treatment of a sphincteric motor dysfunction, the resolution to be preferred is that simplest and most compact, beginning with electrostimulation through a nonjacketing side-entry connector with only an electrical wire, not a fluid drug delivery line or catheter. If inadequate, the addition of a fluid drug delivery line follows. If electrostimulation and direct drug targeting fail, then an electromechanical assist device is employed.

The longitudinal extent of a sphincter usually not affording sufficient space to position both a combination-form electromechanical sphincteric assist device with built in fluid and electrical capability and a nonjacketing side-entry connector, unless confidence in the side-entry connector is high, the nonjacketing side-entry connector should be placed first, just proximal to the sphincter, with the distal end of its catheter and/or electrode set to penetrate the sphincter proper.

Then, if placed, the combination-form electromechanical sphincteric assist device will be drug and electrical discharge capable, allowing the side-entry connector to be removed. If the patient history indicates little probability that the side-entry connector will work to satisfaction, the combination-form electromechanical sphincteric assist device is placed ab initio. The larger sphincters of the digestive and urinary tracts consist of specialized abluminal muscle fibers continuous with the surrounding tissue.

The electromechanical sphincteric assist device is placed to encircle the sphincter, the suture loops 32 such as those shown in FIG. 1 therein used to prevent unwanted mobility, in this case, equivalent to a sliding hiatal hernia. Provided to do so is not likely to result in erosions, ulceration, or fistulization of a sphincter lining such as that of the internal urinary sphincter which is unadapted to and intolerant of constant constriction, the electromagnetic sphincteric assist device type ductus jacket is placed just proximal to the native sphincter. The lining of the digestive tract much tougher and if not so intensely as a sphincter, routinely constrictive, when surrounding tissue or some peculiarity of the anatomy recommend, placement of cardiac, pyloric, and ileocecal electromagnetic sphincters are positioned just proximal or short of the native structure.

The surrounding tissue is dissected away if and only if the placement of an electromagnetic sphincteric assist device has been confirmed as necessary and not likely to cause injury that cannot be controlled through the delivery of medication through an service or accessory channel. Where separation from the surrounding tissue is disruptive, suture loops 32 in the accompanying drawing figures situated about the outer surface of the assist device are used to reattach the surrounding tissue.

The ability to apply any drug, drugs, and/or electrostimulation in any pattern of pulsation with a nonjacketing side-entry connector such as that shown in FIG. 9 and the further ability to mechanically force the motility required with the aid of a combination-form sphincteric ductus side-entry jacket, by its spectrum of treatment modalities and results found empirically through adjustment outside the body, allows dispensing with much prediction and testing to offset the cost of treatment.

While it may be presumed that once forcible closure is instituted, electrical and chemical modulation might just as well be disposed of as superfluous, because forcible closure, especially where the tissue is not adapted for it, often injures the conduit lining. In this circumstance, the sphincteric assist device best includes the capability to forcibly contract the sphincter only once electrical and chemical neuromodulation have been unsuccessful.

For this reason, a sphincteric assist device usually includes electrical and drug delivery means ab initio, allowing the use of force to be minimized through extracorporeal adjustment following closure, without the need for reentry or revision. Then, if neuromodulatory means substantially close the sphincter so that only a final application of constrictive force is necessary to finally squelch acid reflux, the additional force is applied over the shortest interval following neuromodulation.

Thus, if the severity of the condition is recognized early, the placement of a sphincteric assist device with fluid and electrical delivery lines allows one time placement and the ability to adjust the therapy until that regimen most effective with the least treatment is determined. Then if medication and electrostimulation fail, the sphincter is forced shut. A comparable approach applies to the targeted delivery of digestive hormones, enzymes, and electrical neuromodulation to reverse gastric and/or intestinal hypo or hypermotility. The concurrent placement of sensors and control microcontroller allow the process of optimization and future adjustment as necessary to proceed automatically. When placed in conjunction with a robotically assisted procedure, use of a robotic or camera access port already present should be considered.

As to a LeVeen, or peritoneovenous shunt, the ability to access the junction with the vein for delivery of drugs should substantially eliminate the complications of superior vena cava thrombosis, infection, variceal bleeding, and disseminated intravascular coagulopathy encountered with such devices. Ductus and nonjacketing side-entry connectors are intended to remain in place over a long period if not permanently, thus supporting the long term functionality of a fully implanted prosthetic disorder response system that uses inputs from implanted sensors to govern the targeted delivery of drugs to different treatment sites under automatic control. The elimination from the vena cava, internal jugular, or any other vein of an indwelling catheter provides a safety advantage.

Equally important as these conventional applications, the stable connections, long life, and direct to junction delivery of drugs that can treat the disease and maintain the catheteric line means that ductus and nonjacketing side-entry connectors are able to support, and in so doing, make possible, an automatic control system as addressed in copending application Ser. No. 14/121,365, entitled Ductus Side-entry Jackets and Prosthetic Disorder Response Systems, filed on 25 Aug. 2014. Such a system, conceived of as a prosthetic backup immune system able to treat comorbid disease, uses implanted sensors to signal the need to target medication in the appropriate doses to an organ, vessel, or a combination of these.

Full implantation, automatic ambulatory operation, and system self maintenance have the potential to critically improve patient quality of life. Where indwelling catheters limit free movement, require frequent examination, and cause progressive irritation and injury that limit duration, ductus side-entry jackets form secure catheteric junctions with ductus, and nonjacketing side-entry connectors the same with organs and tissues, to minimize if not eliminate growing trauma at the entry wound.

An service or accessory channel to introduce catheter and target maintenance measures as needed is not accessed through a ‘piggyback’ port dangling out through a hole in the body wall but rather through a separate fully implanted or closed-skin portacath or secure port at the body surface, described in copending application Ser. No. 14/121,365. This allows apparatus such as nephrostomy tubes and central venous catheters previously limited to temporary use and the need for replacement if necessary to remain in place over a long term if not permanently. Side-entry connectors are intended for long-term or permanent fastening of synthetic or tissue engineered ductus to or from native or transplanted organ or tissue, thus into the parenchyma.

Ductus side-entry jackets allow secure connection to ductus, and so release, for example, drugs directly into the circulation rather than into the parenchyma. As such, both include secure fastening means, means for passing fluids and/or electrical currents through the junction, and an service or accessory channel to allow release into the line of medication to prevent the buildup of the clot, crystal accretion and biofilm as appropriate which have thwarted the long-term use of narrow catheter implants. Ordinarily, the line proximal to the outlet of the service or accessory channel does not convey biological but rather pharmaceutical materials, so that it is not susceptible to fouling or occlusive buildup.

Ductus and nonjacketing catheter-to-tissue and tissue-to-catheter fasteners that provide secure and leak-free attachment and can be accessed without invasive entry to deliver maintenance substances are indispensable for the implementation of ambulatory prosthetic disorder response systems. If necessary, counteracting agents, such as a solvent or antimicrobial can be included with the pharmaceutical at the inlet into the line. In addition to the growing irritation caused by movement at the tubing-tissue interface, synthetic tubing that is smaller in gauge placed in the vascular tree tends to be thrombogenic and subject to the formation of biofilm, and placed in the urinary tract, susceptible to crystal accretion, as seen in the need to periodically replace current ureteric stents.

However, clot, biofilm, and crystallization eliminated, a synthetic tube can remain in place indefinitely, is not subject to stenosis, degradation, or infection, has no need for a blood supply, has no intrinsic physiology that is mismatched when resituated to a different location, and is not obtained at the cost of a preliminary procedure that harvests and renders normal tissue abnormal. These factors have limited the time that catheters can be allowed to remain in place. Accordingly, side-entry connectors not only pass fluids as a primary object of placement, but incorporate an accessory line for self and catheter support.

Such junctions can be used to extend the indwelling time of catheters in otherwise conventional practice, but are essential for the implementation of automatic ambulatory prosthetic disorder response control systems as described in copending nonprovisional application Ser. No. 14/121,365. Long term stability and ease of maintenance allow, for example, the placement of drug targeting means in a primigravida requiring a drug that would harm the fetus, where the unobtrusive apparatus placed early in pregnancy can remain in place over the balance of her reproductive if not entire life.

Provided a reversal agent is available, incomplete takeup within the target organ or tissue can be accomplished through a second ductus side-entry jacket on the outflow vein or veins to deliver that agent, thus preventing continued transport through the circulation, access to this second jacket as specified above for an service or accessory channel. Essential substances for which there is no reversal agent are prevented from further transport by introducing the medication in the form of a ferrofluid wherein the drug is bound to superparamagnetic nanoparticles drawn by magnets situated about the organ periphery from the point of entry into the surrounding tissue to draw the drug into the parenchyma or surrounding tissue.

Nonjacketing side-entry connectors are of two types, those for internal use as described herein and those for placement at the surface of the body, described in copending application Ser. No. 14/121,365. Reduction in the need for maintenance is advantageous for a patient of any age, but especially for those at the extremes of age and their caregivers. Whereas the object in forming a junction between a synthetic or tissue engineered tube and a native ductus, such as a vessel or a ureter, is to accomplish merging confluence with minimal shear stress, connection to solid or hollow organs and to fascia-invested muscle, for example, is usually to fixedly implant and if necessary, advance and retract a styloid or styliform, that is, a rod or needle-shaped device.

Such include electrodes; ultrasonic, electrohydraulic, and laser probes; scopes; and/or hollow (injection/aspiration) needles, hypotubes, lasers; and/or heating elements. Those implanted for therapeutic neuromodulation can be chemical, electrical, such as leads placed for transcutaneous electrical nerve stimulation, or these inserted side by side. Electrodes, for example, can be electroanalytic and/or electrotherapeutic, such as electroanalgesic, and different styloid or cabled devices can be positioned side by side.

In an automated system, the energization of these, individually or in coaxial or disparate combinations to treat singular or comorbid conditions, can be a part of or coordinated with chemotherapy, radiotherapy, or chemoradiotherapy in adjuvant and/or neoadjuvant relation. That is, a connector for the immobile infixion to or within nontubular or nonductal anatomical structures must allow the connection as necessary of electrical lines and small caliber cabled devices or styliform components such as therapeutic and diagnostic electrodes or microelectrodes, lasers, or probes or microprobes in addition to fluid lines.

The ability to isolate or circumscribe an organ or region for treatment by pharmacotherapy, chemotherapy, radiotherapy (radiation therapy, radiation oncology), or chemoradiotherapy has the potential to eliminate much, perhaps all, of the adverse side effects, drug-drug, and drug-nutrient interactions associated with these treatment modalities. Photon radiation as in brachytherapy involves the infixion of seeds, wires, or pellets that move with the substrate organ or tissue and are therefore positionally stable without the need for a means of positional fixation. Use of a remote afterloader, which has limited applicability, and must be withdrawn leaving no radioactive substance in the patient, denies the ability to terminate the treatment based upon reexamination at intervals without the need to repeat the procedure.

More generally, the ability to isolate a native organ, blood supply territory, or a transplant organ by means of placing side-entry jackets on the arterial inflow, and if necessary, the venous outflow, allows the avoidance of side effects, if any, to the organ or tissue circumscribed, alleviation of such continuous insult alleviating a major contributory factor in rejection. The targeting of a lesion within an organ or tissue is by placing a nonjacketing side-entry connector mounting a styloid device such as a catheter or hollow needle at a fixed angle and depth within the organ or tissue. The use of both jackets and a side-entry connector to treat the same organ or tissue then serves to directly target the lesion while furnishing a background dose to the surrounding tissue as ‘extension for prevention,’ while containing exposure to the tissue intended.

The same application describes radiation shielding with both short and longer half life radionuclides and other radioisotopes (see, for example, Murata, T., Miwa, K., Matsubayashi, F., Wagatsuma, K, Akimoto, K., and 5 others 2014. “Optimal Radiation Shielding for Beta and Bremsstrahlung Radiation Emitted by (89)Sr and (90)Y: Validation by Empirical Approach and Monte Carlo Simulations,” Annals of Nuclear Medicine 28(7):617-622; Bhattacharyya, S. and Dixit, M. 2011. “Metallic Radionuclides in the Development of Diagnostic and Therapeutic Radiopharmaceuticals,” Dalton Transactions 40(23):6112-6128; Yue, K., Luo, W., Dong, X., Wang, C., Wu, G., Jiang, M., and Zha, Y. 2009. “A New Lead-free Radiation Shielding Material for Radiotherapy,” Radiation Protection Dosimetry 133(4):256-260; Amato, E. and Lizio, D. 2009. “Plastic Materials as a Radiation Shield for Beta-Sources: A Comparative Study through Monte Carlo Calculation,” Journal of Radiological Protection 29(2):239-250; Jødal, L. 2009. “Beta Emitters and Radiation Protection,” Acta Oncologica (Stockholm) 48(2):308-313; Papagiannis, P., Baltas, D., Granero, D., Pérez-Calatayud, J., Gimeno, J., Ballester, F., and Venselaar, J. L. 2008. “Radiation Transmission Data for Radionuclides and Materials Relevant to Brachytherapy Facility Shielding,” Medical Physics 35(11):4898-4906; Van Pelt, W. R. and Drzyzga, M. 2007. “Beta Radiation Shielding with Lead and Plastic: Effect on Bremsstrahlung Radiation when Switching the Shielding Order,” Health Physics 92 (2 Supplement): S13-S17).

When flushing through the line with water would not preclude the risk of injury, tungsten shielding offers the best combination of light weight and expense. Tungsten is toxic and must be encapsulated for chemical isolation, polyethylene terephthalate and related polyesters suitable materials therefore. Implants accurately prepositioned to work in conjunction with external pencil beam radiation or other means of excitation from outside the body at intervals, such as radiofrequency magnetic field alternators to warm the implants, can represent strike-target reactive or relay emitter devices, receiving antennas, or discharge tubes for substances used in radiopharmaceutical practice such as nuclides, any of which can be fixedly prepositioned in relation to the target for energization by the external source with the aid of a nonjacketing side-entry connector.

The nonjacketing connectors described herein are intended to achieve positioning as stable and durable as reversibility with relatively little trauma will allow. When placement is temporary, the needles are smooth surfaced and provided with a snare-grab to facilitate extraction. The fine needles must be of extreme strength, hence, made of graphene, titanium, or heat treated 17-4PH and 15-5PH stainless steel, which martensitic however, are magnetic. If this will pose a problem, the needles are made of a cold worked austenitic stainless steel. The use of a nonjacketing side-entry connector assumes that positional stability is essential for a treatment to continue over a period long enough to work at all or to work to better effect.

Scheduled dosing with passive drug delivery necessitates patient or assistant compliance, whereas automated delivery does not. This factor becomes the more important as the number of drugs to be administered increases, especially if the administration thereof must be coordinated. A port with multiple openings fastened to the body surface is not considered an implant. Such a port, described in copending application Ser. No. 14/121,365, entitled Ductus Side-entry Jackets and Prosthetic Disorder Response Systems, can provide openings which are closed off to the exterior by injection bottle cap type elastomeric plugs or a lid that allows insertion of a line from an external pump.

To admit a fiberscope or cabled device such as a fine excimer laser, for example, the plug is withdrawn from the port at the body surface. The direct delivery of drugs to nidi that would ordinarily require passage through the liver is overcome by delivering the drug in its post-liver metabolized form. That is, the liver and/or kidneys bypassed, drugs ordinarily administered as prodrugs must be converted into the biotransformed (post-metabolized, post-hepatic, post-renal—and hypothetically, application to a fetus not yet practicable—post-placental) form exogenously before direct application at the treatment site.

Similarly, for direct application, conventional drugs must be adjusted in dose. Some drugs thought to have no topical or direct effect upon tissue do in fact have such properties, a notable example being statins of which the direct healing effect is referred to as pleiotropic. Using the means described herein, drugs such as lithium, which is neuroprotective (but potentially nephrotoxic) can be directly targeted to the treatment site, pre- or post-hepatically and/or pre- or post-renally.

That the immunosuppressant cyclosporine, also nephrotoxic (see, for example, Yu and Brenner, Op cit., pages 1703-1704), can be targeted to a non-kidney transplant, avoiding the kidney, is further addressed below in this section. With superparamagnetic iron oxide nanoparticles as drug carriers, one can selectively target diseased tissue within an organ or tissue while keeping the agent away from the healthy tissue surrounding the lesion. Such an application was addressed in connection with FIGS. 6, 13A, and 13B in U.S. Pat. No. 11,013,858.

Ordinarily, the use of a reversal agent or counteractant is complicated by the possibility of unwanted reversal; however, the segregation implicit in targeting allows conjugation or chemical reaction between the therapeutic agent and reversal agent to be controlled. Delivery of the therapeutic agent, and if needed, the reversal agent, can be pulsed or continuous. Lithium, for example, to treat bipolar (manic depressive, mood) disorder, which is not metabolized but rather remains intact, can be routed directly to the brain and substantially kept away from the kidneys, intestinal tract, and thyroid, for example.

Targeting to the brain of lithium or another drug, or to the eyes of an ophthalmic drug is by direct delivery into the internal carotids through ductus side-entry jackets or their service or accessory channels. When directly delivered to the brain, the risk of drug-induced renal complications, especially if counteracted when continuing through the circulation, is substantially eradicated. The smaller dose needed when not dispersed throughout the pre- or post-systemic circulation should prove harmless, but if needed, a counteractant, neutralizing, or reversal agent is delivered directly into the jugulars or internal jugulars.

In this way, acetaminophen can be kept away from the kidneys and nonsteroidal anti-inflammatory drugs from the gastrointestinal tract of a patient with chronic or migraine or cluster headache (see, for example, Raskin, N. M. 2005. “Headache,” in Harrison's Principles of Internal Medicine, Op cit., pages 85-94). The direct delivery to the brain of drugs averts metabolism by, and is limited to drugs that do not depend upon, conversion by the liver and kidneys, for example. Such drugs exercise the therapeutic effect locally at the site to which delivered, the brain exemplary in this regard.

Where antecedent conversion of the drug is essential, administration of the drug through direct targeting must deliver the drug in its activated or effective post-metabolized form. For drugs with direct local action, dispersion in a relatively small and substantially isolated volume of blood conserves plasma concentration, minimizes the time to peak plasma concentration as a primary factor in clinical efficacy (Raskin, N. M. 2005, Op cit., page 91), avoids breakdown by nontargeted tissue, and minimizes loss through absorption which could induce adverse side effects.

This consideration, fundamentally important in the administration of chemotherapy, radiotherapy, chemoradiotherapy, and immunotherapy, all inducing severe side effects, is no less important in the administration of migraine medication, where the efficacy of the drug tends to vary in proportion to its toxicity. For example, when dispersed throughout the systemic circulation through injection or oral administration in systemic doses, sumatriptan, usually formulated to include naproxen, one of the most effective drugs for reducing the pain of migraine and one unlike a statin not in question as to its direct tissue contact efficacy, can induce serious side effects, to include ventricular dysrhythmias, coronary vasospasm, myocardial ischemia, and infarction.

Less serious neurological side effects include altered sensation of temperature, pressure, pain, paresthesias, and sleep disturbances. The release of serotonin 1B, 1D receptor agonists, antiemetics, analgesics, for example, to suppress a migraine headache on inception depends upon the experience of an aura or prodrome by a competent patient able to control the drug delivery pump implant. In patients who do not experience an aura, other sensible symptoms, such as paresthesia of a hand that progresses proximally up the arm signals onset (see, for example, The Merck Manual, 2006, page 1848).

In an intellectually impaired adult or a young child, automatic release is effected by a sensor implant which detects a physiological concomitant and experiential correlate to onset, signals the microcontroller to energize the pump, and provides the quantitative information for controlling the pump. Provided distention or vasodilation of the extracerebral cranial arteries signals onset, a thin film strain gauge pressure type sensor implant can be used.

If for any reason, the action of the drug produces results outside the target range, further delivery is stopped upon receipt of pertinent sensor feedback. An unanticipated effect can be encountered during preliminary testing or at any time thereafter in which the patient experiences a primary change in metabolism, disease induced or otherwise. Then delivery of the drug is immediately stopped, and if available, a reversal agent (antidote, counteractant) is delivered. Drug delivery cessation and recovery are the reasons for requiring that all pumps be reversible.

The sensors signal out of range values to their morbidity or organ system control node, whereupon a higher-order controller programmed to coordinate the action of the nodes issues the commands to achieve the most efficacious overall response. The application of such a system is generally reserved for chronic conditions where an automatic system not only effects remedial action immediately to interdict progression but serves to dispel a central condition that detracts from the quality of life. Such an automatic ambulatory system, operating barely if at all noticed, has the potential to forestall if not prevent the inducement by a chronic systemic disease of a terminal condition. For example, if left untreated, diabetes, hypertension, atherosclerosis, or the metabolic syndrome will eventually induce chronic, then end-stage kidney disease.

A suitable circumstance where comorbid disease may be best controlled with automatic monitoring by sensor implants and the delivery of insulin and drugs to treat concurrent hypertension with an angiotensin converting enzyme inhibitor and angiotensin receptor blocker, or atherosclerosis with a statin, is diabetic nephropathy. By impeding progression to end-stage renal disease, which necessitates precise diagnosis and correctly measured treatment, survival is extended (see, for example, The Merck Manual 18th edition, 2006, page 2008). The automatic system functions continuously, and can do so in a mentally impaired patient.

Through the use of catheters made of a hydrophilic materials having a slippery internal surface, usually a fluoropolymer such as polytetrafluoroethylene tubing backed up by at least one service or accessory channel to clean away any buildup of clot, crystal, or biofilm used in accordance with the guidelines set forth in the foregoing and in this application thwarts the use of small gauge synthetic tubing in the body. Additionally, along the vascular tree, an service or accessory channel (service channel, sideline) attached to the primary or mainline is always provided to allow the targeted and tightly metered addition of an anticoagulant, antiseptic, and/or anti-inflammatory as well as any other fluid medication into the blood or therapeutic fluid passing through the mainline.

By substantially avoiding the systemic circulation, the targeted delivery of medication allows use of the drugs at higher concentrations for restricted site specific local application. An automatic ambulatory prosthetic disorder response system with direct and targetable access to multiple sites of internal disease must coordinate the automatic treatment of these in a synchronized manner while the patient engages in normal activity. Even one, much less a collection of indwelling—meaning temporary, nonimplanted—catheters would disallow this. Imperative for the implementation of a fully implanted therapeutic system, safe, secure, and durable pipeline or electrical conductor to tissue connectors were addressed in the copending applications specified.

Already described in application Ser. No. 14/121,365 are body surface ports and ductus side-entry jackets for connection to tubular anatomical structures, or ductus, to meet the immediate requirement for such connection in an automatic ambulatory prosthetic disorder response control system. However, regardless of application thus, such means overcome the need to detain an otherwise ambulatory patient in the clinic merely because a catheter, infusion line, the tape securing it, or the solution used to promote antisepsis require frequent examination and changing or because more radical surgery necessitates more time to heal.

Described here is a prosthetic disorder response system-compatible fluid and electrical line connector for fastening one or a number of catheters to nontubular internal surfaces and organs, such as the kidneys, the urinary or the gall bladder, the spleen, prostate gland, uterus, and any location along a serous membrane-lined internal surface. Surface ports secure the wound at the body surface, ductus side-entry jackets where connection is made to a tubular anatomical structure, and the internal surface connector described herein is used to attach a catheter to any surface which nontubular, is not articulable by means of encirclement.

### SUMMARY OF THE INVENTION

The information handling capability imparted by hierarchical control, previously used to reduce the complexity of decision-making in the fields of robotics, manufacturing, and artificial intelligence is applied to medical diagnostics and therapeutics. In a fully implanted system, sensors positioned to monitor known and predictable secondary or associated disease at the lowest local level, often cellular, input data to nodes or subcontrollers at the same level. Sensors are chosen on the basis of existing and predictable signs and symptoms. These ground level sensors pass their data to diagnostic nodes or controllers at their respective level. Therapy is primarily medicinal but may include electrostimulatory neuromodulation, for example.

At the same time, other ground level sensors strategically positioned in the same or other parts of the body, assigned to monitor the same or an associated or secondary disease process, that is, a comorbidity, likewise send disease-related data to the ground level nodes at their level. Implanted drug reservoirs are preloaded with broad spectrum pharmaceuticals effective over a range of similar, and others most effective in treating specific predictable signs and symptoms.

The nodes at the ground level, one or more in one set assigned to one morbidity and those in another set assigned to another morbidity, pass their data up to a higher cross-morbidity node that identifies medication, for example, that would address the diagnostic data for both morbidities most effectively with the least adverse effects. Where the comorbidities are more than two, the process of coordinating and integrating the indicia associated with additional comorbid disease is likewise diagnosed and passed up to higher level nodes or controllers so that at the highest level, this process integrates the data across the three morbidities.

An implanted microprocessor—the master controller—is programmed to analyze and integrate the highest level, or summary level data, formulate a therapeutic regimen consisting of the fewest drugs in the smallest doses, and where applicable, the energization of electrical therapeutic components, most likely to reinstate homeostasis across the set of comorbidities to the extent possible, then effectuate the response by actuating and metering the ‘stopcocks’ or motors at the outlets of the drug reservoirs to pipe-target the medication according to the resolution arrived at through this process. In so doing, the system reinstates the affected tissue or tissues to the most competent level of performance of which it had been capable before it became affected by disease.

The system can provide a level of performance to compensate for tissue limited by a cytological, histological, or gross anatomical deficiency or malformity that arose during development as results in an inborn error of metabolism, for example. Additionally, such a system is able to compensate for if not restore the level of function of which the structure was capable before having been degraded by disease. Attempting to exceed the level of performance of the system or structure beyond its de facto potential is specifically discounted as injurious. Accordingly, the system detects and responds to the appearance of a disorder or disease process immediately, before the patient becomes aware of it, and reacts to that emergence immediately to optimal effect, the patient ambulatory throughout. The incident can be signaled and transmitted to the clinic telemetrically.

### OBJECTS OF THE INVENTION

The central object of the invention is to provide control means over the automatic detection, diagnosis, and treatment of disease, the semiautomatic execution of solid organ transplantation operations and the semiautomatic detection, diagnosis, and treatment following such operations, and the semiautomatic replacement of congenitally severe malformities of the vasculature to the end that these procedures will demonstrate much greater than conventional durability.

An object of the invention is to provide a fully implanted automatic diagnostic and therapeutic system to evaluate and treat comorbid disease as well as to detect the emergence of and respond to any of a number of predictable intercurrent diseases immediately upon appearance, before symptoms appear or the patient becomes aware of it, in a patient ambulatory and without a loss in freedom of movement, so that diagnosis and treatment are initiated instantly regardless of the time of day, location, or mental state of the patient.

Another object of the invention is to provide a system which can be fully implanted without the need to interrupt the flow of blood through a vessel treated much less induce circulatory arrest with the complications this risks.

Another object of the invention is to provide such a system to administer the transplantation of a solid organ using the compound bypass method and thereafter, provide automatic and immediate followup treatment thereof, as well as respond to post-transplantation complications and predictable intercurrent disease indefinitely, without detracting from the ambulatory state of the patient.

An object of the invention is provide the clinician with the ability to effect the release of drugs and the application of electrostimulatory neuromodulation, for example, anywhere deep inside the body without the need for entry.

Another object is to provide the system in the form of a hierarchical control system wherein different disease processes or comorbidities are specifically and simultaneously addressed at the immediate or ground level by sensors that supply output data to a node or controller at the same level in the hierarchy, other nodes dedicated to monitoring different disease processes then passing their data up to a next higher intermediate node for integrating and generating the best response to the combination of disease processes, this pattern of increased comprehension by passage through higher level nodes of integrated data concerning any additional comorbidities finally presented to a master controller programmed to induce and institute the response best calculated to suppress the combination of disease processes and achieve the condition of optimal homeostasis of which the patient is capable.

Another object of the invention is to isolate the delivery of drugs in separate pipelines each emptying into the blood supply or parenchyma of an organ, gland, or volume of tissue, thereby delivering the complete and proper dose respective of each without the need to compromise due to the potential injury to nontargeted tissue and avoiding the side effects that would be more likely to arise were these drugs released into the circulatory system.

Yet another object of the invention is to provide a fully implanted system of leak-free, durable, and safe drug and blood catheteric pipelines and electrical devices to provide the implanted microcontroller in monomorbid disease and the microprocessor master controller in comorbid disease immediate access to the diseased nidi or tissues, making it possible to directly pipeline-target therapy to any one organ, gland, or tissue.

Another object of the invention is to make possible the coordination, and usually the collocation, of drug need detection and delivery means so that drugs can be targeted directly to the anatomical point of detection or a point functionally related thereto, thereby enabling the implementation of prosthetic disorder response systems, to include those employing hierarchical control.

Yet another object of the invention is to allow the direct and immediate translation of chemical, electrical, and immunoassay feedback diagnostics into automatic drug delivery around the clock, avoiding any impediment to free movement, whether to the locus of detection, the site of the symptom, and/or the etiological origin, under the control of a hierarchical or complex control system capable of predictive or anticipatory control and further adaptable through ‘learning’ ability, and in so doing, apply such control to the practice of internal medicine.

### BRIEF DESCRIPTION OF THE DRAWING FIGURES

FIG. 1 is a schematic, or nonanatomic, representation of a fully implanted automatic ambulatory prosthetic disorder response system showing components always and a few less often needed in such a system, the shown here configured to treat various urological disorders.

FIG. 2 is a schematic, or nonanatomic, representation of a fully implanted system for allowing the wearer to voluntarily divert the outflow of urine from the kidneys with the aid of manually controlled ductus side-entry valves on the ureters to bypass the bladder for outflow directly into a collection bag, the same drainage system equipped with electrically controlled bypass valves when coordination by the automatic implanted diagnostic and therapeutic system shown in FIG. 1 is needed to coordinate the timing of valve opening and closing with the release of drugs into the bladder, the system in FIG. 1 at the same time no less capable of coordinating diagnostic and therapeutic functions appurtenant to other disease processes elsewhere in the body.

FIG. 3 shows the connection of a drug delivery mainline 13, or drugline, and accompanying service or accessory channel, or sideline 11, from a subcutaneously implanted body surface port 16 into which the drug is injected to flow directly into the target ductus 2 shown here as the left anterior descending coronary artery or alternatively into a subcutaneously implanted drug reservoir wherefrom release of the drug is under the control of a fully implanted ambulatory disorder control system such as that shown in FIG. 1, the target ductus as shown being the left anterior descending coronary artery with periodic release of a maintenance statin, for example, periodic, or immediately upon the detection by a sensor incorporated into the side-entry jacket 6 of an incipient parital or complete blockage to the continued flow of blood a thrombolytic.

FIG. 4 shows pumps in a pump-pair wherein drug delivery, or drugline, switching using turrets allows an automatic disorder response system such as that shown in FIG. 1 to index any drug delivery line such as main drugline 13 or service or accessory channel 11 in FIG. 3 into alignment with a pump intake and a drug vial so that any drug in either turret can be released into any pump and any drug delivery line, the pump outlet switching means also shown as a turret but for simplicity, without drug vials.

FIG. 5 is a diagrammatic representation, or schematic, of the control train when a single pump-pair and jacket set, size permitting, is implanted, or if not, inserted in a control, power, and/or pump body pack, shown here in the abstract as to the actual conformation of the parts, the control train comprising a system for the hierarchical control of a prescription-program in accordance with the guidelines set forth by evidence based pharmacy for immediate response to an expression of disease, to include those emergent.

FIG. 6 is a diagrammatic schematic, or circuit diagram, of the interconnections in a hierarchical control system and its positioning as miniaturized for implantation inside or if located outside the body, then relegated to a control, power, and/or pump body pack worn about the waist when a second pump-pair and jacket set to allow any loaded drug to be delivered through any drugline is added to the first.

### DESCRIPTION OF THE PREFERRED EMBODIMENTS OF THE INVENTION

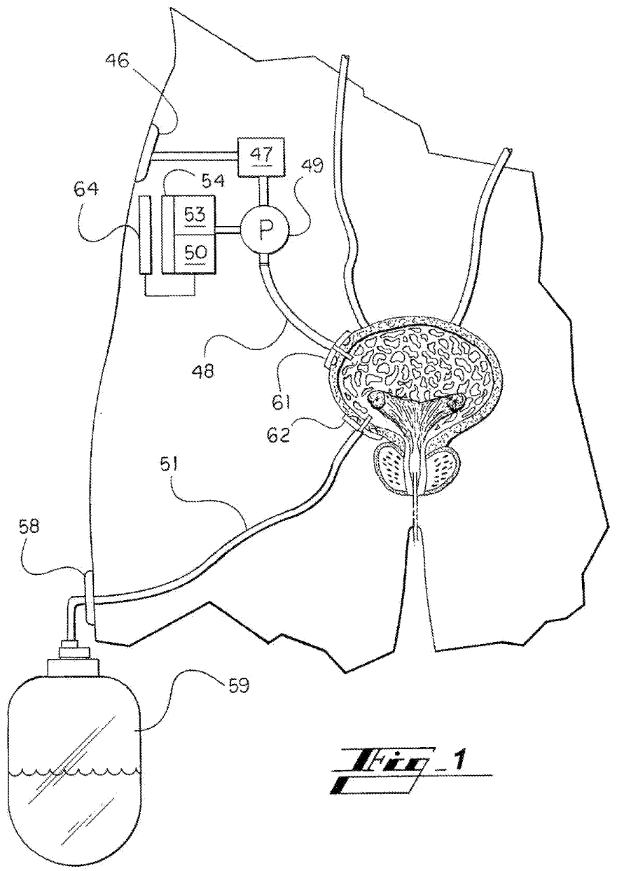

FIG. 1 is a schematic, or nonanatomic, overall representation of a fully implanted prosthetic disorder response system which includes most of the components found in different applications, not all of which will always be needed in any one system. System components are relegated to an paracorporeal, belt-worn body pack only when these are too large or numerous to be implanted. For simplicity and less expense in competent adult patients, such a system is devised to limit the number and type of functions and components and therefore the complexity of the controller and prescription-program to those best if not necessarily automated.

For system simplicity and economy, the release of drugs and/or other therapy such as electrostimulatory for which the need is signaled by the implanted sensors is relegated to the automatic system, whereas scheduled oral medication prescribed for a competent patient is omitted. Automatic response functions include any that demand response to sensor inputs indicating the emergence of an abnormality which had been diagnosed on the basis of a genetic evaluation and the system prepositioned to counteract the condition before the patient becomes aware of it.

For a prescription adherent patient, the functions supplied by the system may be supplemented with an oral or self-injected prescription. FIG. 1 first appeared as FIG. 12A in application Ser. No. 14/998,495, now U.S. Pat. No. 11,013,858, entitled Nonjacketing Side-entry Connectors and pictorial representations and descriptive text explaining the various components used to implement a hierarchical automatic control system and their positioning in the body also appeared earlier in copending application Ser. No. 14/121,365, entitled Ductus Side-entry Jackets and Prosthetic Disorder Response Systems, initially filed on 27 Aug. 2013.

As used here, the control system is the focus, end-connectors, end-effectors, and details concerning component materials and mechanical function omitted as duplicative of information having been set forth earlier in related applications identified in the section above entitled Background of the Invention. The implementation of these components in combination with an automatic ambulatory disorder response system has application to many disorders and disease processes affecting every bodily system, application to the lower urinary tract purely exemplary.

In FIG. 1, part number 46 is a subcutaneously positioned surface port with separate openings into each drug storage reservoir or delivery pipeline shown in FIGS. 26A and 26B of copending application Ser. No. 16/873,914, entitled Vascular Valves and Servovalves—and Prosthetic Disorder Response Systems, of which only drug delivery line 48 is shown here. In FIG. 1, part number 47 represents a drug storage reservoir, shown in FIG. 26B or application Ser. No. 16/873,914.

Body surface ports—even when positioned subcutaneously with a self-sealing cover membrane as provided in subcutaneously positioned portacaths, or mediports, allow the insertion of not just infusion lines but miniature cabled devices for passage through a side-entry jacket for entry into its substrate ductus or side-entry for entry into the substrate parenchyma. Such cabled devices include those both diagnostic and therapeutic, such as lasers, endoscopes, and intravascular ultrasound probes, which allow the application of therapy as well as the withdrawal of biopsy samples.

Depicted in FIG. 1, the body surface port is entirely subcutaneous, or subdermal and entered by inserting a hypodermic needle through the skin and a self-sealing membrane. The position of multiple needle openings would be indicated by tiny tattoo markings which can be lased away were the port removed. However, a body surface port can include one or more above-skin openings when the other openings are subcutaneous. FIG. 26C in copending application Ser. No. 16/873,914 entitled Vascular Valves and Servovalves—and Prosthetic Disorder Response Systems shows a port positioned as is port 58 in FIG. 1 through the center of which passes a urine outflow pipe for attachment to a paracorporeal collection bag cinched about the thigh.

Here in FIG. 1, port 46 in the pectoral region can include one or more above-skin openings surrounded by subcutaneous injection openings where an above-skin opening might serve to admit cabled devices too large in caliber to pass through the skin and port membrane. An above-skin opening can incorporate a universal serial bus socket, for example, to allow access to the implanted microprocessor for adjustments to the prescription-program by the programmer who would plug his keyboard into the socket during an office visit. Reprogramming to any extent can be prerecorded on a USB (jump, flash, keychain) drive for virtually instantaneous entry.

In FIG. 1, part number 48 is a drug delivery pipeline, or drugline; 49 a miniature reversible pump; 50 transdermal charging circuitry; 53 the master controller, or control microprocessor; 54 a rechargeable battery; 58 a body surface port with an outlet to release urine through urine outlet hose 51 into collection bag 59; 61 a nonjacketing side-entry connector that securely connects drugline 48 to the urinary bladder; 62 a nonjacketing side-entry connector that securely connects the bladder to outlet hose 51; and 64 a transdermal, or transcutaneous, battery charging secondary coil.

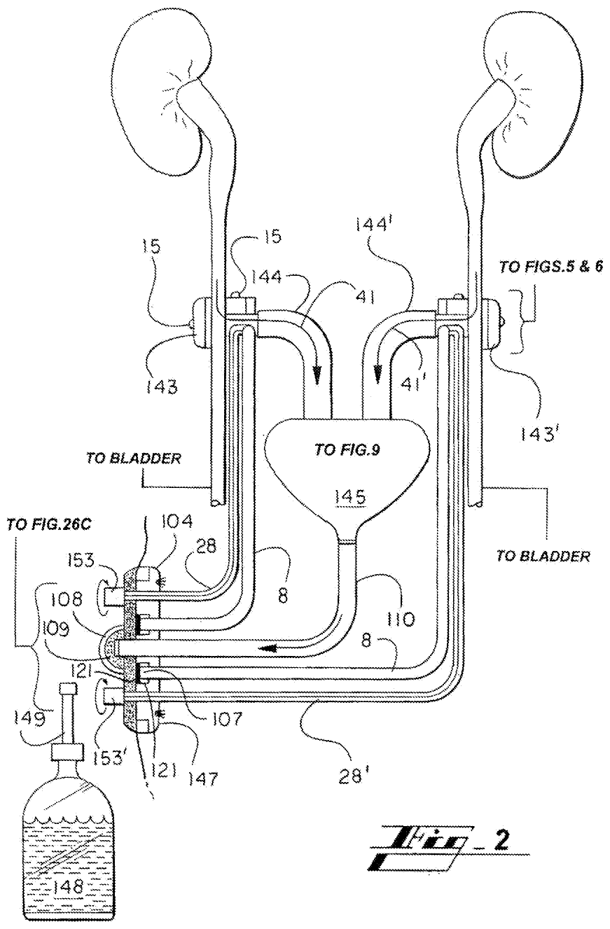

Exceptionally, the arrangement shown in FIG. 2 can be used apart from or in combination with the arrangement shown in FIG. 1 to allow the bladder to remain empty during surgery, treatment, or postsurgical treatment. Because several diagnostic and therapeutic procedures would best be performed and if applicable, allowed to heal with the interior of bladder free of urine, it is significant that the drainage system shown in FIG. 2 allows the bladder to be bypassed.

Significantly, the drainage system shown in FIG. 2, originally shown as FIG. 30 in copending application Ser. No. 16/873,914, entitled Vascular Valves and Servovalves—and Prosthetic Disorder Response Systems. allows the bladder and the rest of the lower urinary tract to be bypassed whenever necessary or desired, whereas a nonadjustable, permanent bladder bypass version of the same system, shown as FIG. 28 in the same application serves as a prosthesis for a patient lacking the lower tract. For the patient with a lower urinary tract, the ability to bypass the bladder and the rest of the track on a voluntary basis not only allows avoiding the disruptions caused by conditions such as frequency, overactive bladder, dysuria, nocturia, urgency, and incontinence regardless of the cause, even neurogenic, and allows the lower tract to be examined and treated when fully drained.

More specifically, FIG. 2 shows a manually switchable bladder bypass for a patient with intractable frequency and/or incontinence whose activities demand the ability to avoid distractions and interruptions due to the need to void. The addition of a prosthetic disorder response system elevates the utility of a lower tract bypass system as shown from the practical to the medically significant in making possible the administration of essential therapy in a dry field while the patient is ambulatory away from the clinic and such treatment proceeds without the conscious awareness of the patient.

Bypass thus allows medication to remain without being washed away and the diseased urothelium to heal without the insult of urine flowing over it. When a drug or drugs for release into the bladder are to coat the interior while the urothelium remains dry, the valves used are not manual but rather solenoid or servo driven, and the microcontroller—or in comorbid disease where the urinary tract is one of multiple affected systems, the master control microprocessor—then automatically times the actuation of the valves to bypass the inflow of urine into a collection bag at an interval preceding the release of the drug or drugs to allow the interior to dry.

The timely administration of drugs, especially when numerous and for release at different related intervals of which some are dependent upon others will be too difficult for many patients who may already be impaired, and a requirement to additionally coordinate this timing with the need to make preparatory adjustments in the delivery mechanism is likely to meet with failure unless such adjustments are automatically coordinated with the release of the various drugs by a controller that governs both. Much the same need for coordination thus applies to the use of other drug-combined means such as electrostimulation, warming, or cooling devices to be actuated in timed relation to the release of drugs or drugs used in combination.

This is significant for facilitating healing following a surgical procedure inside the bladder, for example. The addition of a rudimentary automated system comprising an implanted microcontroller chip and an service or accessory channel on either side would allow the automatically dispensed targeting of a crystal solvent through the valve and into the tract. A more elaborate system would include sensors to detect signs of inflammation, infection, or metaplastic degeneration, for example, and initiate the dispensing of medication while the patient remained ambulatory and unconscious of this action. Copending application Ser. No. 16/873,914, entitled Vascular Valves and Servovalves—and Prosthetic Disorder Response Systems, specifies sensors capable identifying virtually any condition of disease that would affect the urinary tract.

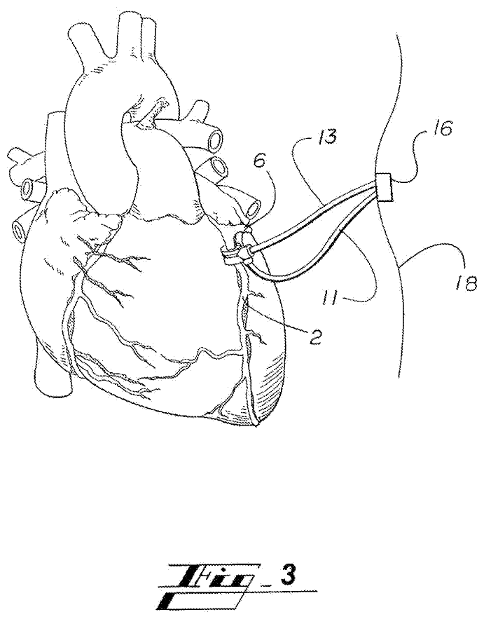

FIG. 3, originally published as FIG. 16 in copending application Ser. Nos. 14/121,365 and 15/998,002 entitled Ductus Side-entry Jackets and Prosthetic Disorder Response Systems, shows the direct connection as exemplary to the left anterior descending coronary artery of a ductus side entry jacket 13 and accompanying service or accessory channel 11 which allows the delivery through the jacket into the substrate artery of liquid drugs. Automated response system sensors incorporated into the jacket are free of the fibrous envelopment to which sensors placed elsewhere are subject, often to the loss of a clear field of view.

An oximetric sensor in this example or with respect to any artery that blocked would lead to a cerebral or myocardial infarction, for example, will instantly detect incipient hypoxia, other sensors capable of detecting the integral stress response and/or ‘cyclokine storm’ associated with the inception of a vasospasm in angina (angina pectoris) or any condition such as the rupture of a vulnerable plaque or thromboembolism associated with an imminent occlusion. As enumerated in copending application Ser. No. 16/873,914, entitled Vascular Valves and Servovalves—and Prosthetic Disorder Response Systems, the state of development in sensors now allows a host of adverse conditions to be detected.

Upon the detection of such an acute incident, a fully implanted disorder response system can immediately pipeline target a concentrated dose of a thrombolytic such as tissue plasminogen activator, uroknase, or streptokinase, to the affected coronary, carotid, or internal carotid artery in an ambulatory patient, and in so doing, interdict the onset of a potentially fatal condition before it has the chance to evolve and before the patient even becomes aware of the condition. Chronic conditions such as stable or unstable angina and cardiac syndrome X are immediately responded to with the direct pipe-targeting into the artery of a vasodilator such as nitroglycerin.

A regular maintenance sensor to detect a chronic condition of atherogenic dyslipidemia rather than an imminent vascular accident monitors the timely dispensing and the effect of maintenance medication such as a statin, low-dose aspirin, clopidogrel, beta blocker, calcium channel blocker, and/or short term acting nitroglycerin. That the onset and duration of action of these drugs is variable and several may be prescribed for timely application means that automatic dispensing will preclude failure to adhere to the prescription despite its complexity or the forgetfulness or the disorientation of an elderly patient, for example.

To conserve energy, where appropriate to the application, sensors are activated intermittently. For system simplicity and reduced expense, newer drugs which are administered by injection in any event, such as proprotein convertase subtilisin/kexin tye 9 (PCSK9) inhibitors to suppress the production of low density lipoprotein currently every two to four weeks with currently experimental variants to be taken as infrequently as twice a year should still be administered through manual injection by a clinician. That is, the automatic system would be capable of administering a drug semiannually, but the increased cost and complexity of the system to incorporate such an infrequently taken drug is best avoided.

The sensor and automatic response remain in place following an angioplasty and stenting to prevent the angina, for example. Copending application Ser. No. 16/873,914, entitled Vascular Valves and Servovalves—and Prosthetic Disorder Response Systems, shows inline coupling jackets which under the control of the automatic disorder response system can replace a segment along a ductus without interfering with the flow therethrough of blood. If vulnerable plaque is situated along a segment of the artery, this segment can be replaced. Otherwise, severe angina is best alleviated by a coronary artery bypass graft.

For immediate response to an emergency signaled to the clinic by implanted sensors that requires a change in the prescription-program, changes are transmitted over the Internet to the unique name of the microprocessor supported by an accompanying mobile hot spot with security provided by a virtual private network, for example. The section above entitled Updating the Prescription-Program deals with remote access to the patient microprocessor by various methods to include remote desktop protocol, or remote computer access with the aid of a virtual private network to protect the privacy of the patient. Intended to represent one in a number of comorbidities, the same arrangement would apply were the condition monomorbid with the system managed by a microcontroller rather than a master control microprocessor in comorbid disease.

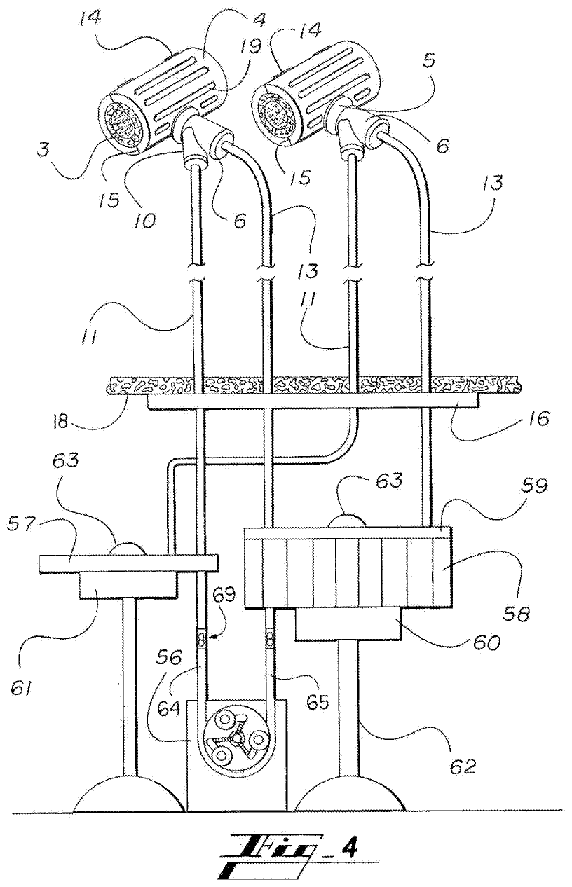

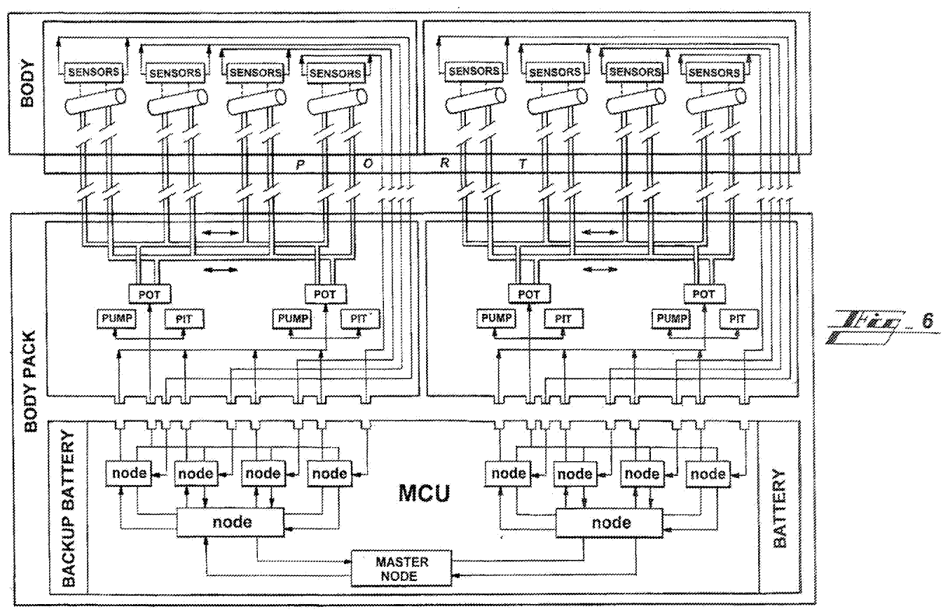

For pictorial clarity, the drug, pump, and drugline mechanism in FIG. 4 is represented in the much larger form intended for incorporation into a belt-worn body pack; the mechanism when implanted considerably miniaturized but essentially the same. To avoid the need to wear a body pack, it preferred that the system be fully implanted; the representation in FIG. 6 of the system components as having been relegated to a body pack rather than implanted indicates that the sizes and/or number of system components were greater than could be implanted.

Such becomes necessary only once the number of components becomes excessive due to the compresence of numerous disorders. Such a condition is not limited to the elderly but may be seen, for example, in the multiply severe comorbid disorders due to congenital, to include pleiotropic origin, in young patients with trisomy mutations. In monomorbid disease with the disorder response system fully implanted, the drug selection mechanism is controlled by a microcontrol chip. In comorbid disease, control is by a microprocessor.

In FIG. 4, providing drugline and drug reservoir vial switching turrets at the intake and outlet lines of each pump in a pair makes it possible to switch the inlets to either or both jackets to any drug loaded. That is, in FIG. 4, each of the pumps in a pump-pair and jacket set is furnished with turrets at both its intake and outlet to allow any drug delivered through the intake turret to be sent to any jacket in the set. FIG. 4 was first published in nonprovisional application Ser. No. 14/121,365 entitled Ductus Side-entry Jackets and Prosthetic Disorder Response Systems, filed on 25 Aug. 2014 following provisional application Ser. No. 61/959,560 filed on 27 Aug. 2013. application Ser. No. 14/121,365 was then updated in continuation-in-part application Ser. No. 15/998,002, filed on 8 Jun. 2018.

In the arrangement depicted in FIG. 4, one of the pumps in a given pump-pair is used independently. The outlet of the other pump in the pump-pair could be plugged into the intake or outlet turret of the other pump; however, the need for such cross-feeding between pumps in a pair is exceptional. Cross-feeding to pumps belonging to other pump-pair and jacket sets is avoided as needlessly complicated as to invite errors. FIG. 4 shows the right-hand pump in a standardized pump-pair wherein drugline switching using turrets allows any drug or line rotated into alignment with the pump intake by the pump intake line switching means shown as a turret to be delivered through any one line rotated into alignment with the pump outlet line by the pump outlet switching means also shown as a turret but without drug vials for simplicity.

FIG. 4 depicts the side-entry connection jacket at the top left as currently connected to pump 56, pump to turret outlet line 64 indexed, or switched by turret 57 motor 61 to the inline position, with accessory or sideline 11 connected to water-jacket or accessory inlet 10 of that jacket. Water jacket 10 assists in preventing extravasation during plug removal and thereafter serves as the accessory inlet to a jacket service or accessory channel which allows the directly piped delivery of drugs and maintenance solutions, for example, into the jacket and the ductus it encircles. The lines of the jacket to the top right are not currently indexed to the pump inline positions and are therefore disconnected from pump 56.

Pump 56 is continuously adjustable in speed and reversible, allowing outflow to and inflow from either jacket over the range of drug volumetric flow rates without the need to switch to lines of different caliber. Pump 56 is usually one of a pair, one pump usually connected to the sideline, that is, the service or accessory channel which allows access to the jacket or connector for the directly targeted delivery of medication or a miniature cabled device such as a laser scope, or intravascular ultrasound probe. When more than one pump-pair is present, the connection of these to either jacket is through lines connected to the turret respective of each jacket.

Reciprocally, jackets not shown in FIG. 4 may communicate with pump 56. The foregoing degrees of flexibility attest to a potential versatility able to respond to extraordinarily complex medical conditions. This potential capability notwithstanding, pump and jacket relations are ordinarily simple. To prevent air from entering the lines in vascular applications, turrets 57 and 59 omit blank vial positions that would leave a line open-ended; and pumping is stopped once the amount of the infusate has passed so that the free end of the line or hose can be disconnected.

As shown, the left-hand turret lacks a vial and reservoir hose plug in table seen at 58 on the right, indicating that in this application, only the right-hand turret loads drug vials or receives medicated hydrogel or other therapeutic substance reservoir lines or hoses. Were, however, drugs to be supplied from the turret to the left or a tacky medicinal hydrogel, for example, to be recirculated through the closed pump circuit with pump 56 when rotated clockwise, then the turret on the left would be of the same kind as that on the right. If to fill the line then stop or recirculate the gel, a reservoir hose would supply the gel necessary to fill the line. Segments along a line of medicinal or nonmedicinal gel or water can be interposed between segments of the primary medicinal as a way to deliver the primary medicinal in an intermittent manner.

Control of this rotating turret mechanism is one means by which the master controller can position drugs for release to specific targets, alternative embodiments such as miniaturized functionally equivalent. To conserve space, drugs are moved through narrow gauge druglines, often conventional catheters. If the distance to the target makes it necessary, the drug can be diluted or positioned at the head of a column of gel or water. This application concerned with control of a totally implanted disorder response system, the review is necessarily cursory, a more thorough description of drug delivery mechanisms provided in copending application Ser. No. 15/998,002.

FIG. 4 shows one of the two pumps in a pump-pair with switching mechanisms at both the pump intake and outlet to allow the sequential delivery of any drug to the mainline or sideline of any jacket. Accordingly, FIG. 4 shows the right-hand pump in a standardized pump-pair wherein line switching using turrets allows any drug or line rotated into alignment with the pump intake by the pump intake line switching means shown as a turret to be delivered through any one line rotated into alignment with the pump outlet by the pump outlet switching means, also shown as a turret, but again without drug vials for simplicity.

In FIG. 4, crushed tacky hydrogel, drugs, drug hydrogels, and/or wash water for separate consecutive delivery to different jackets are delivered from one of the pumps in a pump-pair through the lines 13 and 11 and side-entry connector 6 of either jacket. Pump outlet flow lines (arms, runs) 11 are connected at intervals about outflow indexing turret plate 57, and pump intake lines 13 are connected at intervals about turret drug vials and/or vials used as drug reservoir hose connectors to pump intake sectional tray consisting of sectional tray 58 and hold-down plate 59. Each turret rotates one inlet vial or line into the in-line position at the same time that it rotates the preceding line out of the in-line position. Lines 13 and 11 are given enough slack that these do not interfere with rotation of the turrets.

Also not shown are service or accessory channels to deliver an anticoagulant such as a heparin or thrombolytic drip to prevent the accumulation of a residue along the inner wall of the druglines, or of clot when the fluid moved is blood. In FIG. 4, part number 3 is a viscoelastic polyurethane foam jacket lining with surface coated to prevent dissolution essential to prevent compression of the vasa vasora and vasa nervora as would induce atherosclerotic degeneration. Part number 4 a strong jacket outer shell or casing made of polyether ether ketone (PEEK) or another biocompatible nonallergenic material such as gear grade nylon with edges rounded to prevent irritation to surrounding tissue.

Part number 5 is a the outer sealing grommet cap of an eccentric bushing that allows the razor sharp circle cutter, or trepan, at the end of the bushing facing into the jacket, hidden in this view but clearly shown in copending application Ser. No. 15/998,002, entitled Ductus Side-entry Jackets and Prosthetic Disorder Response Systems filed on 25 Aug. 2014, to be rotated and reciprocated to expedite removal from the side of the ductus of a plug of tissue to serve as the ostium of sideline 13 exiting side connector, side-stem, or mainline 6, ordinarily fed by a subsidiary sideline shown here as part number 10.

Jacket side-stem, or side-connector 6 shown here ensheaths mainline 13 here used as a drugline leading to the turret aligned drug reservoir vial 58; part number 10 is a side-stem subsidiary takeoff, or sidestem, that ordinarily conveys a drugline or service or accessory channel; 11 used to empty into service or accessory channel; 13, the jacket mainline having emerged through side-stem, or side-connector 6, used here as the drugline connecting the jacket to the turret aligned drug reservoir vial 58.

Part number 14 is a spring-hinge which urges the jacket shut but not with a restorative force so great as to prohibit growth in a young child; 15 indicates the position of the joint separating the spring-loaded semicylindrical halves of the jacket opposite to the jacket spring-hinges; 16 schematically represents the subcutaneously implanted body surface port through drugs and therapeutic solutions are replenished regardless of the number or size of the components needed as too numerous or large for the system to be fully implanted so that the internally unaccommodable components had to be relegated to a body pack. Part number 18 schematically represents the body surface integument, comprising the skin, subcutaneous fascia, and fat.

Part number 19 points to side-entry jacket and lining through and through slits to allow open exposure of a sufficient area of the vascular adventitia of the encircled artery as essential to preclude the complete enclosure of its nervelets and tiny vessels as would induce atherosclerotic degeneration; 56 is the right hand of two drug delivery pumps, shown here as peristaltic, or of the roller type, for propelling drugs from the drug reservoir 58 and through sideline, or service or accessory channel 11.

Part number 57 is the drug pump outflow indexing turret plate; 58 is the drug turret drug reservoir or vial storage tray that rotates to index the required drug vial into alignment with line 65 leading to drug pump 56 as the drug pump intake line; 59 is the pump intake drug vial hold-down plate, which along with drug storage vial sectional tray 58, comprises the drug pump intake turret; 60 is the drug inlet turret motor of the right-hand pump shown.

Part number 61 is the drug outlet turret motor for the right-hand pump shown; 62 is the right hand drug turret stile or mounting shaft; 63 is the drug vial hold-down plate retainer cap; 64 is the drug pump outlet line that leads into the sideline or service or accessory channel 11; 65 is the pump drug intake line from drug storage vial 58; and 69 are fluid line cleanouts. An additional service or accessory channel feeding into the druglines to drip in an anticoagulant or thrombolytic to prevent the formation of clot is not integral to the mechanism is not shown.

Whereas bedridden patients need drugs to be pumped to the target, in an ambulatory patient able to maintain an upright posture, drug reservoirs can allow the drug to flow down to the target due to gravity. Generally, it is simpler and less costly to move expensive drugs through druglines in the form of a diluted continuous column rather than to arrange for a much smaller concentrated amount of the drug to be driven down the drugline ahead of a column of water by the reservoir pump. That is, the degree of control, precision calibration of the componentry, expense, and susceptibility to malfunction to provide such head of water column drug-water reservoir switching and apportioning are greater than is the use of narrow gauge druglines and diluted drugs of like dose as were these highly concentrated and positioned ahead of a column of water.

Moreover, that the continuous input of sensor feedback as medication is added is the basis for determining whether the optimal dose has been delivered means that the gradual rate of drug delivery is easier to determine than would be the sudden delivery of the drug. Nevertheless, if excessive water or dilution are problematic, the alternative of delivery at the head of a column of water which upon delivery is stopped leaving almost all of the water in the line is to be preferred. Depending upon the connections made between pumps and jackets, a pump or pump-pair can support one or more side-entry jackets, and more than one pump-pair can support a single jacket. In FIG. 4, the ductus side-entry jackets and lines at the top of the figure are described in detail in copending application Ser. No. 15/998,002 entitled Ductus Side-entry Jackets and Prosthetic Disorder Response Systems where the same drawing figure appears as FIG. 32 and part numbers of the mechanism are identified and explained in greater detail.

When too small to provide the volume of medication required, the standardized drug vial shown in FIGS. 33 thru 36 of copending application Ser. No. 15/998,002 for insertion into a turret drug vial receptacle, represented here as part number 58, serves as the connector attached to the end of a drug delivery line from the drug reservoir for engagement in the turret. The vial also provides the initial dose of the drug or another drug preparatory to delivery of the primary drug. A more usual and versatile arrangement is shown here in FIG. 4, wherein one of the pumps in a pump-pair and jacket set is furnished with turrets at both its intake and outlet to allow any drug delivered through the intake turret to be sent to any jacket in the set.

The two jackets represented in FIG. 4 as equal in size and distance from the pump might be placed along the same ductus, or ductus differing not only in size and/or distance from the pump but belonging to different bodily systems and therefore be assigned to different arms in the hierarchical control system. This might, for example, consist of a jacket placed along the digestive tract and another placed about the artery that supplies that segment of the tract, or each jacket might treat different diseases whether related or coincidental. Flexibility and speed in reconnection of the lines to and from each pump are often significant when line switching must be reconfigured quickly as might arise in the testing undergone during installation.

Whereas lines supporting side-entry connection jackets placed along the vascular tree or the urogenital tract are small enough in caliber that placement should seldom encroach upon neighboring tissue as to cause pain by compression of a nerve or vessel, larger jackets positioned along the gastrointestinal tract or airway might do so. Where anatomical or operative considerations discourage the placement of multiple lines to access a given jacket, the input line to each jacket is provided with a conventional miniature piggyback port with valve. Encroachment upon neighboring tissue is to be avoided. All jackets have their edges and corners rounded, If necessary, a polymeric gas-permeable cushion not subject to enzymatic or hydrolytic breakdown can be glued to the jacket to serve as a cushion between it and the neighboring anatomy.

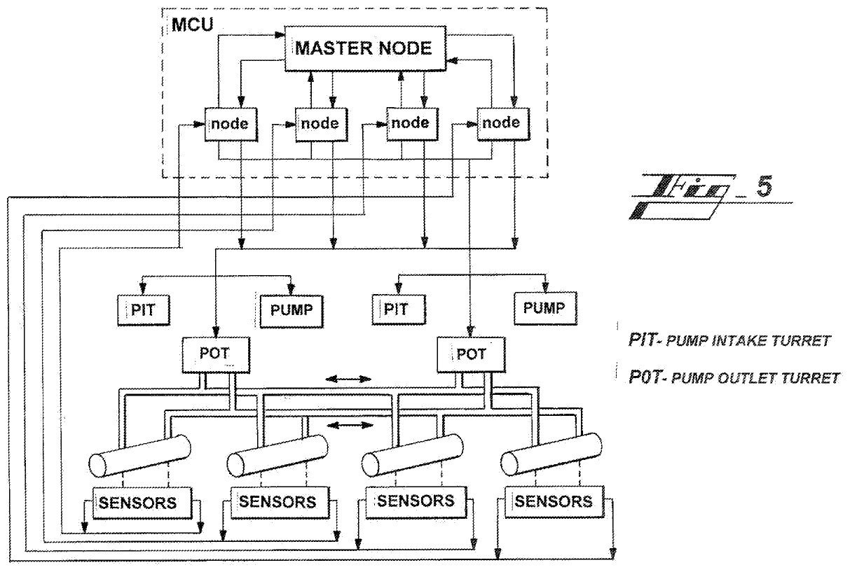

FIG. 5 provides a schematic of the control hierarchy for a single pump-pair in support of four jackets in the pump-pair and jacket set, the control program, that is, the prescription-program, of the master node, a microprocessor, determined by the specific or comorbid conditions to be treated. Nodes subordinate to the master node are generally microcontroller chips. FIG. 5 provides a schematic of the pump-pack, jacket set, and control system. In FIG. 5, only the control train is represented, the distinction between intra and extracorporeal elements omitted.

A paracorporeal such as a waist-worn body pack affords considerably more space and can hold numerous drugs, other therapeutic agents, and equipment maintenance solutions in relatively large volumes. While depicted with full-sized components as relegated to a body pack, the control hierarchy is always microminiaturized and therefore implantable with the impediment of a pack eliminated. FIG. 5 is a diagrammatic representation of the control train when a single pump-pair and jacket set is implanted or inserted in a pump body pack, shown here therefore, in the abstract as to whether the system components are positioned inside or outside the body. As shown, the control trains in FIGS. 5 and 6 comprise relatively simple hierarchical control systems.

Accordingly, FIG. 5 provides a schematic of the control hierarchy for a single pump-pair in support of four jackets consisting of the pump-pair and jacket set, connecting fluid and electrical lines when not wireless, the subsidiary node microcontroller chips, and master control microprocessor which as the system master, integrates and coordinates the information received from the subsidiary nodes and administers the prescription-program.

The number of symptom or disease process axes (channels, arms) of nodes at the ground level, usually incorporated into their respective end-connectors, and the number of levels rising up from the ground level progressively more inclusive and integrative of the data passed up to them, hence, the number of levels in the tree are determined by the number of comorbidities or symptoms to be treated. Unlike FIG. 6, which provides a schematic diagram of the pump-pack, jacket set, and control system, in FIG. 5, only the control train is represented, the distinction between intra- and paracorporeal elements not indicated.

More specifically, FIG. 6 is a simplified schematic or circuit diagram of the interconnections among the nodes in a hierarchical control system and the positioning of system components as implanted or outside the body such as when a second pump-pair and jacket set is added to the first in the pump-pack. An extracorporeal pack affords considerably more space and can hold a larger volume of numerous drugs, other therapeutic agents and equipment maintenance solutions, the need therefor mostly applicable to the elderly prescribed polypharmacy. The control hierarchy itself comprises microcontrollers and a master control microprocessor which tiny, are implanted with the impediment of a pack eliminated. A given hierarchy can be embodied in a single microchip.

In FIG. 6, the distinction between system components which are fully, or closed-skin, implanted and others which are relegated to a usually belt-worn body pack is evidence that in this patient, the comorbidities and/or the expressions of each in different tissues were more numerous and complex than could have been diagnosed and responded to by a system comprised of components all of which would have been fully and yet comfortably implanted. For the medical need to preclude the implantation of the entire system should arise only in complex comorbid disease, and when it does, the body pack should be made as small and lightweight as possible to be a minimal impediment to freedom of movement.

When implanted, the contents labeled body pack at the lower left in FIG. 6 are miniaturized; otherwise, FIG. 6 applies no less to a fully implanted as to a body worn, or paracorporeal pack carry system. Also when implanted, to preclude complications due to encroachment upon or strangulation of tissue by wires, data intercommunication from the sensors and subordinate nodes and control signals from the master node are preferably by wireless, or Bluetooth transmission. For pictorial clarity, where the electrical and fluid lines between nodes and jackets are actually separate and distinct, those between nodes and jackets are shown as consolidated until finally led to each jacket, and remote sensors and auxiliary drug supply reservoirs have been omitted. Electrical connectors, more remote sensors, drug supply reservoirs and outlet pumps controlled by the master node have been omitted.

FIG. 6 includes both the system components implanted—jackets, sensors, fluid lines, control electronics, and so on—and those relegated to the pump-pack when number and/or size of these makes a paracorporeal complement unavoidable. Depending upon the size and weight the patient is likely to tolerate, a paracorporeal pack affords considerably more space and can hold a larger volume and number of drugs, other therapeutic agents, and equipment maintenance solutions. Not all system components able to be situated outside the body, the impediment of a body pack is to be avoided whenever possible. When implanted, the contents labeled body pack at the lower left in FIG. 6 are miniaturized; otherwise, FIG. 6 applies no less to a fully implanted as to a body pack carried supplementary system.

Fluid and electrical connections between the implanted and pack-relegated components are conventional, numerous like situations—ventricular assist devices, artificial hearts—having set the precedent. In FIG. 6, such connections are schematically represented as plugs and sockets that appear much as a square wave. To preclude complications due to encroachment upon or strangulation of tissue by wires, data intercommunication from the sensors and subordinate nodes and control signals (but not power, which is delivered by hard wire or transcutaneous energy transfer to the inmate battery of each end effector) from the master node are preferably wireless, such as by Bluetooth transmission, the respective targets distinguished by carrier frequency.

In FIGS. 5 and 6, single lines are electrical, or if it is found difficult to route the electrical lines without the risk of strangulating intervening structures, then connected by wireless, such as Bluetooth, transmission rendered selective by difference in carrier frequency with power transferred to component inmate batteries by transcutaneous energy transfer. If virtually simultaneous operation is essential but cannot be achieved with a single carrier switched among the jackets, then the microprocessor is provided with more than one transmitter.

Further for visual clarity, where the electrical and fluid lines between nodes and jackets are actually separate and distinct, those between nodes and jackets are shown as consolidated until finally led to each jacket, and remote sensors and auxiliary drug supply reservoirs have been omitted. Electrical connectors, more remote sensors, drug supply reservoirs and outlet pumps controlled by the master node have been omitted. If provided with the requisite switching and valving, the fluid and electrical lines shown as shared could support each jacket independently but not simultaneously, the utility thereof contingent upon the condition or conditions to be treated; simultaneous capability is accomplished by furnishing the components necessary.

While the ultimate object here is to provide a completely implanted disorder and disease or comorbid disease response system which functions autonomously and silently in a fully ambulatory patient oblivious to its operation where the occasional need for a supplementary body pack is unavoidable, nothing said here should be misconstrued as discounting an embodiment comprising an extracorporeal console inclusive of the control, power, and drug storage and selection components where only the end-connectors, end-effectors, and connecting lines have been implanted for connection to the console through a body surface multiconductor receptacle. Whether configured to respond to conditions of disease, disorder, and/or use to administer an organ transplant, for example, such consoles can be portable or kept in the clinic.

1. A fluid drug and electrical stimulation delivery system comprising ductus connectors that outflow into the blood supply of any specific solid organ or gland, and extravascular tissue connectors that outflow to any depth into any specific volume of tissue, said connectors constructed to remain in place indefinitely, and configured for use as components in a fully implanted automatic control system.

2. A fluid drug delivery system comprising an indexing mechanism which rotates each of a number of fluid drug containers into position to release a fluid drug into a fluid drug delivery pipeline, said fluid drug delivery pipelines configured to empty through a stationary leak-free connector into the blood supply of a specific targeted organ or gland, or into a volume of tissue affected by a disease process; wherewith said stationary leak-free connector blocks out all other tissue from the path and the target of fluid drug delivery.

3. An automatic diagnostic and therapeutic prescription control system to serve as an automatic prosthetic disorder response comprising a fluid drug selection mechanism such as a turret to introduce one of a number of fluid drugs into a fluid drug delivery pipeline wherein said fluid drug delivery pipeline comprises a terminus comprising a stationary leak-free connector; wherein said connector is configured to be connected to the blood supply of a specific organ or gland or volume of tissue of the site of a disease, wherein said diagnostic system comprises sensors positioned at the primary and secondary sites of symptoms associated with the disease; wherein said diagnostic system is programmed to indicate on the basis of physiological response negative feedback from said sensors, the data stored in the memory of the controller, and the fluid drugs provided to the system, the drug and dose thereof which are most efficacious in treating the disease, and using said drug selection mechanism to treat said disease in accordance with said most efficacious drug in the most efficacious dose.

4. The implanted automatic disorder response system of claim 3 of which only components too large or numerous to be implanted are relegated to a paracorporeal body pack.

5. The automatic diagnostic and therapeutic prescription control system of claim 3, wherein said fluid drug delivery pipeline is closed off to all parts of the body except the blood supply or parenchyma of a site of disease.

6. The automatic diagnostic and therapeutic prescription control system of claim 3 wherein said fluid drug delivery pipeline empties into or admits the fraction of the general circulation supplying said organ, gland, or volume of tissue of the site of a disease so that the ascertainment of maximum efficacy of the targeted drug or drugs must take into account the interactions among all the drugs targeted and in the general circulation in the doses of each present.

7. The automatic diagnostic and therapeutic prescription control system of claim 3 configured to record and retain the fluid drug selection device address and dose of the fluid drugs which had been most efficacious in treating a site of disease for later application.

8. The automatic diagnostic and therapeutic prescription control system of claim 3 wherein said system is organized hierarchically, so that an implanted master control microprocessor programmed to respond to the diagnostic and therapeutic information necessary to treat each of a number of symptoms appurtenant of a plurality of comorbidities can at a first level of diagnostic and therapeutic microcontroller node input in a hierarchical tree of such microcontroller nodes, evaluate each symptom-assigned sensor input data, pass this initial-level evaluation up to a next higher level of microcontroller nodes which then generate a therapeutic evaluation inclusive of the morbidities passed up to these to optimize the treatment for both each and the set of morbidities passed up to that level in the hierarchical tree, this data passed up through the tree to yet more inclusive node evaluators to a master control microprocessor for execution of its prescription-program by translating the sum of data needed to the optimal net therapy across the combination of morbidities to most closely reinstate normal homeostasis.

9. For a patient with plural comorbidities requiring treatment with multiple drugs and/or electrostimulation, a hierarchical control program for execution by an implanted microprocessor which uses inputs from implanted symptom-sensors which pass diagnostic and therapeutic data up through a decision tree of microcontroller node chips to generate more comorbidity symptom-inclusive data as the next higher level of these rises, to diagnose and optimize therapy to achieve optimal homeostasis across the entire set of said comorbidities for the medication made available to it.

10. An automatic diagnostic and therapeutic prescription control system which fully implanted, allows fluid drugs injected through a subcutaneously implanted port with multiple openings to be stored in subcutaneously implanted fluid drug reservoirs for release through fluid drug delivery pipelines each respective of a fluid drug reservoir, wherein said port is entered through a self-puncture resealing entry diaphragm, and the outlet of each fluid drug reservoir is connected to an outlet pump, wherein said outlet pump empties into the fluid drug pipeline respective of that fluid drug reservoir, wherein said fluid drug delivery pipelines each terminate at a different site of disease or into the general circulation through a stationary leak-free connector, wherein said reservoir outlet pumps are actuated by the system master control microprocessor executing its prescription-program.

11. The subcutaneously implanted port according to claim 10 comprising a self-resealing puncture entry diaphragm and openings leading directly or through drug reservoirs into different fluid drug delivery lines, the lumina thereof configured to pass through miniature diagnostic and therapeutic cabled devices such as scopes, lasers, intravascular ultrasound probes, and thrombectomizers to a site of disease or its blood supply, said line when connected to a large vein also capable of serving to make possible intravenous delivery of total parenteral nutrition, chemotherapy, antibiotics and other drugs, as well as to allow the withdrawal of blood and tissue biopsy samples.

12. The subcutaneously implanted port according to claim 10, further comprising one or more electrical sockets connected to conductors for energizing electrically powered therapeutic devices each such device controlled by the same controller as controls the release and doses of drugs to treat symptoms of the same disease process on the basis of disease analyte sensor feedback, wherein the output of said sensors is passed to a prescription-programmed microelectronic controller; wherein the microelectronic controller actuates said electrically powered therapeutic devices at each such site apart from or in coordination with concurrent drug delivery as necessary.

13. A combination of fluid drug delivery pipelines for direct delivery of fluid drugs from subcutaneously implanted fluid drug reservoirs; wherein said fluid drug delivery pipelines are replenished through a body surface port having openings respective of each of said drug reservoirs for delivery of said drugs into diseased tissue or the blood supply thereof; wherein said drug delivery lines are otherwise closed off from the circulatory system, further comprising a microelectronic controller and sensors; said microelectronic controller executing a pharmaceutical prescription-program responsive to sensor inputs upon which basis said implanted controller sets the doses for release to each site of disease by controlling the outlet pump of each said fluid drug reservoir.

14. A controlled fluid drug and electrical stimulation therapy delivery system comprising i) a closed system of fluid pipelines for direct delivery of medicinal fluids, ii) fluid drug reservoirs accessed by said fluid pipelines through a body surface port, iii) secure end-connectors; wherein said medicinal fluid is delivered into diseased tissue or its blood supply through secure end-connectors; iv) electrical conductors for energizing electrically powered therapeutic devices, v) analyte sensors configured to detect the need for medicinal, electrostimulatory, and thermal therapy, and vi) a prescription-programmed microelectronic control system controller; wherein each device is directed toward the same site of disease as the disease analyte sensor or sensors respective of each; wherein the data collected by said sensors is transmitted to its respective prescription-programmed microelectronic control system controller in a rising hierarchical tree; which coordinates the inputs from the different symptom sensors to include additional symptoms as the tree is ascended to that of a master control microprocessor to direct the release of medication to and actuate said therapeutic devices at each site to achieve the optimal result across the combination of symptoms.

15. The controlled fluid drug and electrical stimulation therapy delivery system according to claim 14 wherein said control system is organized in the form of a hierarchical tree of microcontroller nodes of increasing purview moving up said tree, so that an implanted master control microprocessor programmed with the diagnostic and therapeutic information necessary to treat any symptoms in a number of comorbidities can, at a first level of diagnostic and therapeutic nodes in the hierarchy, evaluate sensory data pertaining to each symptom of each morbidity, pass the evaluated sensory data up to a second level of nodes wherein the combination of therapeutic measures is optimized to cover both morbidities, wherein this pattern of more inclusive data processing is continued up said tree to include all of the symptoms to be treated, whereupon summary data is generated and is passed to the master control microprocessor to translate the sum of data in accordance with its prescription-program into the net therapy that most closely approximates normal homeostasis across the combination of morbidities.

16. The controlled fluid drug and electrical stimulation therapy delivery system according to claim 14 or claim 15, further comprising a plurality of ductus side-entry jackets and nonjacketing side-entry connectors wherewith at least one pump supplying fluid drugs to these and/or an electrical discharge therapeutic device is controlled by a microprocessor according to a prescription-program; wherein a plurality of disease associated physiological indicia acting as symptom sensors implanted at different locations in the body send outputs which the microcontroller nodes at the lowest level report up through the tree as negative feedback to signal out of the normal range conditions to the next higher level microcontroller nodes in the hierarchical tree conformed control system; wherein the microcontroller nodes pass their information up to microcontroller nodes at the next higher level in the tree until at the highest level, a master control microprocessor responds according to its prescription-program by returning a response signal back down through the chain of successive nodes to cause said pumps to index to and release the drugs prescribed for the symptoms in the doses commanded and to effect the discharge of electrical current as necessary to return said physiological indicia back to within the normal range thus minimizing if not eliminating said symptoms.

17. The hierarchically controlled fluid drug and electrical stimulation therapy delivery system according to claim 14 or claim 15, wherein the hierarchical process applied to coordinate the diagnosis and treatment of multiple symptoms in comorbid disease effectuates responsive action which achieves the optimal response for each symptom so that the sum thereof manifests normal homeostasis or as close thereto as the means made available to the system will allow.

18. The hierarchically controlled fluid drug and electrical stimulation therapy delivery system according to claim 14 or claim 15, wherein the hierarchical process applied to coordinate the treatment of multiple symptoms in comorbid disease if prevented from attaining a better resolution of a symptom due to a less than adequate drug it has been provided resorts to a subroutine with drug reference memory to identify a replacement drug best suited to correct the condition.

19. An automatic disorder response system comprising a plurality of implanted sensors, sensor data responsive microcontrollers at different levels of sensor data coordination, and means for the direct pipeline targeting of fluid drugs and electrostimulation to the sites of disease, wherein each sensor is directed to a symptom in a number of symptoms due to concurrent disease processes of which those sensors directed to the symptoms attributable to any one disease process transmit their data to higher level microcontrollers dedicated to each disease process to determine the fluid drug, drugs, and/or electrostimulators most efficacious for the treatment thereof and this data is passed up to higher level microcontrollers that coordinate the data of subordinate microcontrollers to include that pertinent to progressively more symptoms and disease processes, which pass their data to a master control microprocessor that integrates the sum of data provided to it and uses said data as the basis for commanding specific corrective measures it commands to ameliorate the combination of disease processes individually and together.

20. The automatic disorder response system of claim 19 wherein the assessment of overall drug and electrostimulatory efficacy is determined by the highest level controller on the basis of negative feedback at the microcontrollers at each of the levels of increasing inclusivity subordinate to it and therefor at its own summary level of the effect of each drug and/or electrostimulatory for its respective symptom and the combination thereof for the sum of symptoms which most closely approximates the overall effect desired.

21. The system of claim 19 where said implanted automatic disorder response system is organized in the form of a pyramidal tree comprising microcontroller nodes that deliver more inclusive information at each higher level in said tree, wherein at the highest level, a master control node—in comorbid disease, a microprocessor—accepts the sensory data coordinated and accumulated by the microcontroller nodes subordinate to it and returns motor commands that proceed in the opposite direction down said tree to control the system end-effectors, to include miniature peristaltic pumps or electrical stopcocks at the outlet of implanted drug reservoirs as well as electrostimulatory end-effectors as appropriate, said sensors continuously monitoring and reporting the responsive action up through the tree of microcontroller nodes to the master controller as it occurs so that said tree finds the optimal doses of drugs by negative feedback as a whole.

22. The automatic disorder response system of claim 19 wherein the effect of each drug on its respective symptom is considered independently by an initial level sensor or sensors and microcontroller node assigned to said symptom, the effect of said drug made evident as negative feedback in symptom alleviation responsive to the application of said drug, this information used to minimize the dose of said drug, and this ascertainment of amelioration passed up to a next higher level microcontroller node, which receiving this data and that negative feedback data from another drug directed to another symptom, generates an assessment of efficacy of these drugs when used together, this pattern of increasing inclusivity continued by being passed up to a number of higher level microcontroller nodes of which the number of levels is determined by the number of drugs used, this pattern culminating in input for integration and the identification of any adverse interactions among the drugs with the other drug on the basis of drug interaction data stored in the read only memory of the master control microprocessor, which then returns that combination of drug release and electrostimulation signals back down this control tree to the drug outlet release motors and electrostimulators associated with each symptom to dispense the optimized therapy for the combination of symptoms to be treated.

23. An automatic disorder response system according to claim 19 or claim 22 wherein the release of fluid drugs is exclusively through catheteric pipelines which isolate as said pipelines convey more than a single drug targeted to the same organ, gland, or volume of tissue directly into the blood supply or parenchyma of said organ, gland, or volume of tissue, the catheteric isolation of drugs from one another thus minimizing side effects provoked than were said fluid drugs dispersed throughout the circulatory system so that nontargeted tissue would be adversely exposed to said fluid drug, where the effect and doses of said drugs is continuously monitored by sensors dedicated to said organ, gland, or volume of tissue which transmit their data to organ symptom-dedicated microcontrollers and a master microprocessor to optimize the dose of each drug.

24. An automatic disorder response system according to claim 19 or claim 22 wherein the release of fluid drugs includes both direct release into the circulation and release through catheteric pipelines which isolate as said these convey more than a single drug targeted to the same organ, gland, or volume of tissue directly into the blood supply or parenchyma of said organ, gland, or volume of tissue, said control system configured to optimize the relative concentrations in the drugs piped and those not to obtain the best outcome for the combination of drugs used.

25. A fully implanted automatic disorder response system that coordinates the data provided by a plurality of implanted sensors, each sensor assigned to one or more symptoms of one or more disease processes, each such sensor continuously transmitting its data pertaining to the change in symptom status responsive to the release of a drug, this information passed up to a ground level microcontroller node in a rising hierarchical decision tree, said ground level microcontroller node and another adjacent to it aimed at another symptom of the same or another disease process to which another drug was directed in turn passing their data up to a next higher, cross-level microcontroller node to evaluate the efficacy of the two drugs working together, this pattern continued up to the next level microcontroller node of which each level represents the addition of another drug to one and same or different disease processes in order to provide a master control microprocessor at the head of the tree with the information necessary to determine which fluid drugs in the fewest number and smallest dose and which nondrug effectors such as electrostimulatory and thermal, acting together should optimally affect the combination of symptoms and the combined efficacy of the drugs when released together to elicit the optimal effect over the combination of disease processes and thus most closely reinstate normal homeostasis, said master control microprocessor using this information as a continuous input of negative feedback at every node in the tree to command the actuation of the motors controlling the outlets of implanted fluid drug reservoirs to release the drugs and implanted electrostimulation devices to discharge current thereby to attain the optimal therapeutic effect.

26. An implanted automatic disorder response system comprising sensors aimed at the symptoms of a disease process, wherein said sensors continuously input data indicating the instantaneous status of said symptoms to a dedicated microcontroller chip, wherein other such sensor-microcontroller chip pairs are assigned to other symptoms of disease processes, wherein both such sensor-microcontroller chip pairs input their data for coordination to a higher level microcontroller chip for determining the best combination of drugs isolated from one another by direct pipeline-targeting into the blood supply or parenchyma of each such disease site to treat the sum of said disease processes.

27. An implanted automatic disorder response system comprising sensors aimed at the symptoms of a disease process, wherein said sensors continuously input data indicating the instantaneous status of said symptoms to a dedicated microcontroller chip, wherein other such sensor-microcontroller chip pairs are assigned to other symptoms of disease processes, wherein both such sensor-microcontroller chip pairs input their data for coordination to a higher level microcontroller chip for determining the best combination of drugs isolated from one another by release into the general circulation to treat the sum of said disease processes.

28. An implanted automatic disorder response system comprising sensors aimed at the symptoms of a disease process, wherein said sensors continuously input data indicating the instantaneous status of said symptoms to a dedicated microcontroller chip, wherein other such sensor-microcontroller chip pairs are assigned to other symptoms of disease processes, wherein both such sensor-microcontroller chip pairs input their data for coordination to a higher level microcontroller chip for determining the best combination of drugs isolated from one another by direct pipeline-targeting and drugs released into the blood supply or parenchyma of each such disease site to treat the sum of said disease processes.

29. A prosthetic disorder response system which includes an epicutaneous body surface port incorporating a data transmission socket such as a universal serial bus or standard telephone port to allow a prescription-programmer to plug in a code transmission device such as a computer or universal serial bus flash drive in order to enter updates to the prescription-program during and in response to the diagnostic findings obtained during an office visit.

30. A prosthetic disorder response system incorporating a totally implanted digital drug release and electrostimulation remediation command-issuing controller, in comorbid disease, a microprocessor, capable of Internet-implemented data transmission and reception with virtual private network capability for security, said digital controller programmed to transmit out-of-range physiological patient sensor data for which it had not been provided the means of reversal and remediation to the clinic and receive responsive adjustments to the prescription-program from a remote prescription-programmer to change the onboard prescription-program in response to the emergency condition even before the patient becomes conscious of the condition.
